SEQUENCE LISTING (180 sequences)

SEQ ID NO: 1 - protein, 12 aa, synthetic construct
  REGION 1..12: note = Synthetic Construct
SYPSNALSLHKY

SEQ ID NO: 2 - protein, 12 aa, synthetic construct
  REGION 1..12: note = Synthetic Construct
TLGLRPVPVATT

SEQ ID NO: 3 - protein, 12 aa, synthetic construct
  REGION 1..12: note = Synthetic Construct
YDAIQRPTGQLS

SEQ ID NO: 4 - protein, 12 aa, synthetic construct
  REGION 1..12: note = Synthetic Construct
HSPTTSLTLSYK

SEQ ID NO: 5 - protein, 12 aa, synthetic construct
  REGION 1..12: note = Synthetic Construct
ADSNHAYERDSV

SEQ ID NO: 6 - protein, 12 aa, synthetic construct
  REGION 1..12: note = Synthetic Construct
AESPLTNRGWNP

SEQ ID NO: 7 - protein, 12 aa, synthetic construct
  REGION 1..12: note = Synthetic Construct
AHPHSDKLVPPR

SEQ ID NO: 8 - protein, 12 aa, synthetic construct
  REGION 1..12: note = Synthetic Construct
AHSSFGFSHLAV

SEQ ID NO: 9 - protein, 12 aa, synthetic construct
  REGION 1..12: note = Synthetic Construct
AHVNSSERIHPY

SEQ ID NO: 10 - protein, 12 aa, synthetic construct
  REGION 1..12: note = Synthetic Construct
ANTELALANRKH

SEQ ID NO: 11 - protein, 12 aa, synthetic construct
  REGION 1..12: note = Synthetic Construct
APNVGDLTSLLG

SEQ ID NO: 12 - protein, 12 aa, synthetic construct
  REGION 1..12: note = Synthetic Construct
AQSTTMSTSQRN

SEQ ID NO: 13 - protein, 12 aa, synthetic construct
  REGION 1..12: note = Synthetic Construct
ARSLEPAPSRHS

SEQ ID NO: 14 - protein, 12 aa, synthetic construct
  REGION 1..12: note = Synthetic Construct
ATPGSFQTIRAN

SEQ ID NO: 15 - protein, 12 aa, synthetic construct
  REGION 1..12: note = Synthetic Construct
AMNMRSLTYSDL

SEQ ID NO: 16 - protein, 12 aa, synthetic construct
  REGION 1..12: note = Synthetic Construct
APQTSDSTLTVM

SEQ ID NO: 17 - protein, 12 aa, synthetic construct
  REGION 1..12: note = Synthetic Construct
CVTPSNRDCSHS

SEQ ID NO: 18 - protein, 12 aa, synthetic construct
  REGION 1..12: note = Synthetic Construct
EALLKPYSFAYP

SEQ ID NO: 19 - protein, 12 aa, synthetic construct
  REGION 1..12: note = Synthetic Construct
EHVDMWNLVSAS

SEQ ID NO: 20 - protein, 12 aa, synthetic construct
  REGION 1..12: note = Synthetic Construct
EKYPPSSMDQRS

SEQ ID NO: 21 - protein, 12 aa, synthetic construct
  REGION 1..12: note = Synthetic Construct
ELVSSSDQRNKN

SEQ ID NO: 22 - protein, 12 aa, synthetic construct
  REGION 1..12: note = Synthetic Construct
FPKSVYPAMPRP

SEQ ID NO: 23 - protein, 12 aa, synthetic construct
  REGION 1..12: note = Synthetic Construct
FSPTQANTIHRW

SEQ ID NO: 24 - protein, 12 aa, synthetic construct
  REGION 1..12: note = Synthetic Construct
FTNPFGPYPTGR

SEQ ID NO: 25 - protein, 12 aa, synthetic construct
  REGION 1..12: note = Synthetic Construct
GKQTLHSFATHW

SEQ ID NO: 26 - protein, 12 aa, synthetic construct
  REGION 1..12: note = Synthetic Construct
GPWLGSNMRGAS

SEQ ID NO: 27 - protein, 12 aa, synthetic construct
  REGION 1..12: note = Synthetic Construct
GRDMPMSALMRH

SEQ ID NO: 28 - protein, 12 aa, synthetic construct
  REGION 1..12: note = Synthetic Construct
GSAARTISPSLL

SEQ ID NO: 29 - protein, 12 aa, synthetic construct
  REGION 1..12: note = Synthetic Construct
GSMFYLPMPERG

SEQ ID NO: 30 - protein, 12 aa, synthetic construct
  REGION 1..12: note = Synthetic Construct
GTDIIHPRVIFN

SEQ ID NO: 31 - protein, 12 aa, synthetic construct
  REGION 1..12: note = Synthetic Construct
GTASRTHSYYSL

SEQ ID NO: 32 - protein, 12 aa, synthetic construct
  REGION 1..12: note = Synthetic Construct
HNTPMLDSRGNN

SEQ ID NO: 33 - protein, 12 aa, synthetic construct
  REGION 1..12: note = Synthetic Construct
HPALSQHLGPVA

SEQ ID NO: 34 - protein, 12 aa, synthetic construct
  REGION 1..12: note = Synthetic Construct
HTPHPGGRSVPP

SEQ ID NO: 35 - protein, 12 aa, synthetic construct
  REGION 1..12: note = Synthetic Construct
HYKPHVSSLPLA

SEQ ID NO: 36 - protein, 12 aa, synthetic construct
  REGION 1..12: note = Synthetic Construct
IGSKSPLRLTMD

SEQ ID NO: 37 - protein, 12 aa, synthetic construct
  REGION 1..12: note = Synthetic Construct
KLTTDKVRTITL

SEQ ID NO: 38 - protein, 11 aa, synthetic construct
  REGION 1..11: note = Synthetic Construct
LIAGWDISSKR

SEQ ID NO: 39 - protein, 12 aa, synthetic construct
  REGION 1..12: note = Synthetic Construct
LPKVSVPRHPSV

SEQ ID NO: 40 - protein, 12 aa, synthetic construct
  REGION 1..12: note = Synthetic Construct
LTPHLGTHKSTT

SEQ ID NO: 41 - protein, 12 aa, synthetic construct
  REGION 1..12: note = Synthetic Construct
MKAHHSQLYPRH

SEQ ID NO: 42 - protein, 12 aa, synthetic construct
  REGION 1..12: note = Synthetic Construct
MNIAELRNSDLN

SEQ ID NO: 43 - protein, 12 aa, synthetic construct
  REGION 1..12: note = Synthetic Construct
NANHNPLNLKSA

SEQ ID NO: 44 - protein, 12 aa, synthetic construct
  REGION 1..12: note = Synthetic Construct
QFAKTSDPGSLT

SEQ ID NO: 45 - protein, 12 aa, synthetic construct
  REGION 1..12: note = Synthetic Construct
QIFNHSPNDPKK

SEQ ID NO: 46 - protein, 11 aa, synthetic construct
  REGION 1..11: note = Synthetic Construct
QYVPYLPPAIL

SEQ ID NO: 47 - protein, 12 aa, synthetic construct
  REGION 1..12: note = Synthetic Construct
NNTDHRQLTSTT

SEQ ID NO: 48 - protein, 13 aa, synthetic construct
  REGION 1..13: note = Synthetic Construct
NYLPHQSSSPSRG

SEQ ID NO: 49 - protein, 11 aa, synthetic construct
  REGION 1..11: note = Synthetic Construct
QYVPYLPPAIL

SEQ ID NO: 50 - protein, 11 aa, synthetic construct
  REGION 1..11: note = Synthetic Construct
RIPMPSYMNHM

SEQ ID NO: 51 - protein, 13 aa, synthetic construct
  REGION 1..13: note = Synthetic Construct
SDASGIPKRLAFP

SEQ ID NO: 52 - protein, 12 aa, synthetic construct
  REGION 1..12: note = Synthetic Construct
SDTTSKHLYTRL

SEQ ID NO: 53 - protein, 12 aa, synthetic construct
  REGION 1..12: note = Synthetic Construct
SGYSAEGGKPVL

SEQ ID NO: 54 - protein, 11 aa, synthetic construct
  REGION 1..11: note = Synthetic Construct
SGYSQGGKPVL

SEQ ID NO: 55 - protein, 12 aa, synthetic construct
  REGION 1..12: note = Synthetic Construct
SHGISSTPPGQA

SEQ ID NO: 56 - protein, 12 aa, synthetic construct
  REGION 1..12: note = Synthetic Construct
SLPLAIHNSRPN

SEQ ID NO: 57 - protein, 12 aa, synthetic construct
  REGION 1..12: note = Synthetic Construct
SLTDYVRKGPRI

SEQ ID NO: 58 - protein, 12 aa, synthetic construct
  REGION 1..12: note = Synthetic Construct
SNPSAHKDDSKR

SEQ ID NO: 59 - protein, 12 aa, synthetic construct
  REGION 1..12: note = Synthetic Construct
SRYMGPLDLLAP

SEQ ID NO: 60 - protein, 12 aa, synthetic construct
  REGION 1..12: note = Synthetic Construct
SRYMGPLDLLAP

SEQ ID NO: 61 - protein, 12 aa, synthetic construct
  REGION 1..12: note = Synthetic Construct
SSMPINSPATRQ

SEQ ID NO: 62 - protein, 12 aa, synthetic construct
  REGION 1..12: note = Synthetic Construct
SVDYSFSNRTDR

SEQ ID NO: 63 - protein, 12 aa, synthetic construct
  REGION 1..12: note = Synthetic Construct
SYHLSNTFRVQA

SEQ ID NO: 64 - protein, 12 aa, synthetic construct
  REGION 1..12: note = Synthetic Construct
TAVLAPQPWLNL

SEQ ID NO: 65 - protein, 12 aa, synthetic construct
  REGION 1..12: note = Synthetic Construct
TGSAKFLQRDTH

SEQ ID NO: 66 - protein, 12 aa, synthetic construct
  REGION 1..12: note = Synthetic Construct
TKPSVVHWPMIR

SEQ ID NO: 67 - protein, 12 aa, synthetic construct
  REGION 1..12: note = Synthetic Construct
TPHGYQPMQGKT

SEQ ID NO: 68 - protein, 12 aa, synthetic construct
  REGION 1..12: note = Synthetic Construct
TQGSGFSSPILP

SEQ ID NO: 69 - protein, 12 aa, synthetic construct
  REGION 1..12: note = Synthetic Construct
TTDFFYKRTFFT

SEQ ID NO: 70 - protein, 12 aa, synthetic construct
  REGION 1..12: note = Synthetic Construct
TTRPNHVHLAKI

SEQ ID NO: 71 - protein, 12 aa, synthetic construct
  REGION 1..12: note = Synthetic Construct
VAQRHTLTSITV

SEQ ID NO: 72 - protein, 12 aa, synthetic construct
  REGION 1..12: note = Synthetic Construct
VDAKYGRHIPPV

SEQ ID NO: 73 - protein, 12 aa, synthetic construct
  REGION 1..12: note = Synthetic Construct
VPTQWTHRESHA

SEQ ID NO: 74 - protein, 12 aa, synthetic construct
  REGION 1..12: note = Synthetic Construct
WGVTKPIRTSTL

SEQ ID NO: 75 - protein, 12 aa, synthetic construct
  REGION 1..12: note = Synthetic Construct
WGVTKPIRTSTL

SEQ ID NO: 76 - protein, 11 aa, synthetic construct
  REGION 1..11: note = Synthetic Construct
WYPSNALSLHK

SEQ ID NO: 77 - protein, 12 aa, synthetic construct
  REGION 1..12: note = Synthetic Construct
YASQSALSHSAR

SEQ ID NO: 78 - protein, 12 aa, synthetic construct
  REGION 1..12: note = Synthetic Construct
YVKSHTTTAVRQ

SEQ ID NO: 79 - protein, 4 aa, synthetic construct
  REGION 1..4: note = Synthetic Construct
GGGS

SEQ ID NO: 80 - protein, 6 aa, synthetic construct
  REGION 1..6: note = Synthetic Construct
GGGSGG

SEQ ID NO: 81 - protein, 8 aa, synthetic construct
  REGION 1..8: note = Synthetic Construct
GGGSGGGS

SEQ ID NO: 82 - protein, 15 aa, synthetic construct
  REGION 1..15: note = Synthetic Construct
GGGGSGGGGSGGGGS

SEQ ID NO: 83 - protein, 17 aa, synthetic construct
  REGION 1..17: note = Synthetic Construct
GGGGSGGGGSGGGGSGG

SEQ ID NO: 84 - protein, 20 aa, synthetic construct
  REGION 1..20: note = Synthetic Construct
GGGGSGGGGSGGGGSGGGGS

SEQ ID NO: 85 - protein, 15 aa, synthetic construct
  REGION 1..15: note = Synthetic Construct
ADSNHAYERDSVGGG

SEQ ID NO: 86 - protein, 15 aa, synthetic construct
  REGION 1..15: note = Synthetic Construct
AESPLTNRGWNPGGG

SEQ ID NO: 87 - protein, 15 aa, synthetic construct
  REGION 1..15: note = Synthetic Construct
AHVNSSERIHPYGGG

SEQ ID NO: 88 - protein, 15 aa, synthetic construct
  REGION 1..15: note = Synthetic Construct
ANTELALANRKHGGG

SEQ ID NO: 89 - protein, 15 aa, synthetic construct
  REGION 1..15: note = Synthetic Construct
APNVGDLTSLLGGGG

SEQ ID NO: 90 - protein, 15 aa, synthetic construct
  REGION 1..15: note = Synthetic Construct
AQSTTMSTSQRNGGG

SEQ ID NO: 91 - protein, 15 aa, synthetic construct
  REGION 1..15: note = Synthetic Construct
ARSLEPAPSRHSGGG

SEQ ID NO: 92 - protein, 15 aa, synthetic construct
  REGION 1..15: note = Synthetic Construct
ATPGSFQTIRANGGG

SEQ ID NO: 93 - protein, 15 aa, synthetic construct
  REGION 1..15: note = Synthetic Construct
CVTPSNRDCSHSGGG

SEQ ID NO: 94 - protein, 15 aa, synthetic construct
  REGION 1..15: note = Synthetic Construct
ELVSSSDQRNKNGGG

SEQ ID NO: 95 - protein, 15 aa, synthetic construct
  REGION 1..15: note = Synthetic Construct
GPWLGSNMRGASGGG

SEQ ID NO: 96 - protein, 15 aa, synthetic construct
  REGION 1..15: note = Synthetic Construct
GSAARTISPSLLGGG

SEQ ID NO: 97 - protein, 15 aa, synthetic construct
  REGION 1..15: note = Synthetic Construct
GSMFYLPMPERGGGG

SEQ ID NO: 98 - protein, 15 aa, synthetic construct
  REGION 1..15: note = Synthetic Construct
GTASRTHSYYSLGGG

SEQ ID NO: 99 - protein, 15 aa, synthetic construct
  REGION 1..15: note = Synthetic Construct
HPALSQHLGPVAGGG

SEQ ID NO: 100 - protein, 15 aa, synthetic construct
  REGION 1..15: note = Synthetic Construct
HTPHPGGRSVPPGGG

SEQ ID NO: 101 - protein, 15 aa, synthetic construct
  REGION 1..15: note = Synthetic Construct
IGSKSPLRLTMDGGG

SEQ ID NO: 102 - protein, 15 aa, synthetic construct
  REGION 1..15: note = Synthetic Construct
KLTTDKVRTITLGGG

SEQ ID NO: 103 - protein, 14 aa, synthetic construct
  REGION 1..14: note = Synthetic Construct
LIAGWDISSKRGGG

SEQ ID NO: 104 - protein, 15 aa, synthetic construct
  REGION 1..15: note = Synthetic Construct
LPKVSVPRHPSVGGG

SEQ ID NO: 105 - protein, 15 aa, synthetic construct
  REGION 1..15: note = Synthetic Construct
MKAHHSQLYPRHGGG

SEQ ID NO: 106 - protein, 14 aa, synthetic construct
  REGION 1..14: note = Synthetic Construct
QYVPYLPPAILGGG

SEQ ID NO: 107 - protein, 15 aa, synthetic construct
  REGION 1..15: note = Synthetic Construct
NNTDHRQLTSTTGGG

SEQ ID NO: 108 - protein, 15 aa, synthetic construct
  REGION 1..15: note = Synthetic Construct
NYLPHQSSSPSRGGG

SEQ ID NO: 109 - protein, 14 aa, synthetic construct
  REGION 1..14: note = Synthetic Construct
QYVPYLPPAILGGG

SEQ ID NO: 110 - protein, 15 aa, synthetic construct
  REGION 1..15: note = Synthetic Construct
SNPSAHKDDSKRGGG

SEQ ID NO: 111 - protein, 15 aa, synthetic construct
  REGION 1..15: note = Synthetic Construct
SRYMGPLDLLAPGGG

SEQ ID NO: 112 - protein, 15 aa, synthetic construct
  REGION 1..15: note = Synthetic Construct
SVDYSFSNRTDRGGG

SEQ ID NO: 113 - protein, 15 aa, synthetic construct
  REGION 1..15: note = Synthetic Construct
SYHLSNTFRVQAGGG

SEQ ID NO: 114 - protein, 15 aa, synthetic construct
  REGION 1..15: note = Synthetic Construct
SYPSNALSLHKYGGG

SEQ ID NO: 115 - protein, 15 aa, synthetic construct
  REGION 1..15: note = Synthetic Construct
TLGLRPVPVATTGGG

SEQ ID NO: 116 - protein, 15 aa, synthetic construct
  REGION 1..15: note = Synthetic Construct
TTDFFYKRTFFTGGG

SEQ ID NO: 117 - protein, 15 aa, synthetic construct
  REGION 1..15: note = Synthetic Construct
TTRPNHVHLAKIGGG

SEQ ID NO: 118 - protein, 15 aa, synthetic construct
  REGION 1..15: note = Synthetic Construct
VAQRHTLTSITVGGG

SEQ ID NO: 119 - protein, 15 aa, synthetic construct
  REGION 1..15: note = Synthetic Construct
VPTQWTHRESHAGGG

SEQ ID NO: 120 - protein, 15 aa, synthetic construct
  REGION 1..15: note = Synthetic Construct
WGVTKPIRTSTLGGG

SEQ ID NO: 121 - protein, 15 aa, synthetic construct
  REGION 1..15: note = Synthetic Construct
YDAIQRPTGQLSGGG

SEQ ID NO: 122 - protein, 16 aa, synthetic construct
  REGION 1..16: note = Synthetic Construct
AHPHSDKLVPPRGGGS

SEQ ID NO: 123 - protein, 16 aa, synthetic construct
  REGION 1..16: note = Synthetic Construct
AHSSFGFSHLAVGGGS

SEQ ID NO: 124 - protein, 16 aa, synthetic construct
  REGION 1..16: note = Synthetic Construct
AMNMRSLTYSDLGGGS

SEQ ID NO: 125 - protein, 16 aa, synthetic construct
  REGION 1..16: note = Synthetic Construct
APQTSDSTLTVMGGGS

SEQ ID NO: 126 - protein, 16 aa, synthetic construct
  REGION 1..16: note = Synthetic Construct
EALLKPYSFAYPGGGS

SEQ ID NO: 127 - protein, 16 aa, synthetic construct
  REGION 1..16: note = Synthetic Construct
EHVDMWNLVSASGGGS

SEQ ID NO: 128 - protein, 16 aa, synthetic construct
  REGION 1..16: note = Synthetic Construct
EKYPPSSMDQRSGGGS

SEQ ID NO: 129 - protein, 16 aa, synthetic construct
  REGION 1..16: note = Synthetic Construct
FPKSVYPAMPRPGGGS

SEQ ID NO: 130 - protein, 16 aa, synthetic construct
  REGION 1..16: note = Synthetic Construct
FSPTQANTIHRWGGGS

SEQ ID NO: 131 - protein, 16 aa, synthetic construct
  REGION 1..16: note = Synthetic Construct
FTNPFGPYPTGRGGGS

SEQ ID NO: 132 - protein, 16 aa, synthetic construct
  REGION 1..16: note = Synthetic Construct
GKQTLHSFATHWGGGS

SEQ ID NO: 133 - protein, 16 aa, synthetic construct
  REGION 1..16: note = Synthetic Construct
GRDMPMSALMRHGGGS

SEQ ID NO: 134 - protein, 16 aa, synthetic construct
  REGION 1..16: note = Synthetic Construct
GTDIIHPRVIFNGGGS

SEQ ID NO: 135 - protein, 16 aa, synthetic construct
  REGION 1..16: note = Synthetic Construct
HNTPMLDSRGNNGGGS

SEQ ID NO: 136 - protein, 16 aa, synthetic construct
  REGION 1..16: note = Synthetic Construct
HSPTTSLTLSYKGGGS

SEQ ID NO: 137 - protein, 16 aa, synthetic construct
  REGION 1..16: note = Synthetic Construct
HYKPHVSSLPLAGGGS

SEQ ID NO: 138 - protein, 16 aa, synthetic construct
  REGION 1..16: note = Synthetic Construct
LTPHLGTHKSTTGGGS

SEQ ID NO: 139 - protein, 16 aa, synthetic construct
  REGION 1..16: note = Synthetic Construct
MNIAELRNSDLNGGGS

SEQ ID NO: 140 - protein, 16 aa, synthetic construct
  REGION 1..16: note = Synthetic Construct
NANHNPLNLKSAGGGS

SEQ ID NO: 141 - protein, 16 aa, synthetic construct
  REGION 1..16: note = Synthetic Construct
QFAKTSDPGSLTGGGS

SEQ ID NO: 142 - protein, 16 aa, synthetic construct
  REGION 1..16: note = Synthetic Construct
QIFNHSPNDPKKGGGS

SEQ ID NO: 143 - protein, 15 aa, synthetic construct
  REGION 1..15: note = Synthetic Construct
RIPMPSYMNHMGGGS

SEQ ID NO: 144 - protein, 16 aa, synthetic construct
  REGION 1..16: note = Synthetic Construct
SDASGIPKRLAFPGGS

SEQ ID NO: 145 - protein, 16 aa, synthetic construct
  REGION 1..16: note = Synthetic Construct
SDTTSKHLYTRLGGGS

SEQ ID NO: 146 - protein, 16 aa, synthetic construct
  REGION 1..16: note = Synthetic Construct
SGYSAEGGKPVLGGGS

SEQ ID NO: 147 - protein, 15 aa, synthetic construct
  REGION 1..15: note = Synthetic Construct
SGYSQGGKPVLGGGS

SEQ ID NO: 148 - protein, 16 aa, synthetic construct
  REGION 1..16: note = Synthetic Construct
SHGISSTPPGQAGGGS

SEQ ID NO: 149 - protein, 16 aa, synthetic construct
  REGION 1..16: note = Synthetic Construct
SLPLAIHNSRPNGGGS

SEQ ID NO: 150 - protein, 16 aa, synthetic construct
  REGION 1..16: note = Synthetic Construct
SLTDYVRKGPRIGGGS

SEQ ID NO: 151 - protein, 16 aa, synthetic construct
  REGION 1..16: note = Synthetic Construct
SSMPINSPATRQGGGS

SEQ ID NO: 152 - protein, 16 aa, synthetic construct
  REGION 1..16: note = Synthetic Construct
SYPSNALSLHKYGGGS

SEQ ID NO: 153 - protein, 16 aa, synthetic construct
  REGION 1..16: note = Synthetic Construct
TAVLAPQPWLNLGGGS

SEQ ID NO: 154 - protein, 16 aa, synthetic construct
  REGION 1..16: note = Synthetic Construct
TGSAKFLQRDTHGGGS

SEQ ID NO: 155 - protein, 16 aa, synthetic construct
  REGION 1..16: note = Synthetic Construct
TKPSVVHWPMIRGGGS

SEQ ID NO: 156 - protein, 16 aa, synthetic construct
  REGION 1..16: note = Synthetic Construct
TLGLRPVPVATTGGGS

SEQ ID NO: 157 - protein, 16 aa, synthetic construct
  REGION 1..16: note = Synthetic Construct
TPHGYQPMQGKTGGGS

SEQ ID NO: 158 - protein, 16 aa, synthetic construct
  REGION 1..16: note = Synthetic Construct
TQGSGFSSPILPGGGS

SEQ ID NO: 159 - protein, 16 aa, synthetic construct
  REGION 1..16: note = Synthetic Construct
VDAKYGRHIPPVGGGS

SEQ ID NO: 160 - protein, 15 aa, synthetic construct
  REGION 1..15: note = Synthetic Construct
WYPSNALSLHKGGGS

SEQ ID NO: 161 - protein, 16 aa, synthetic construct
  REGION 1..16: note = Synthetic Construct
YASQSALSHSARGGGS

SEQ ID NO: 162 - protein, 16 aa, synthetic construct
  REGION 1..16: note = Synthetic Construct
YDAIQRPTGQLSGGGS

SEQ ID NO: 163 - protein, 16 aa, synthetic construct
  REGION 1..16: note = Synthetic Construct
YVKSHTTTAVRQGGGS

SEQ ID NO: 164 - protein, 18 aa, synthetic construct
  REGION 1..18: note = Synthetic Construct
GSTSGSGKPGSGEGSTKG

SEQ ID NO: 165 - protein, 39 aa, synthetic construct
  REGION 1..39: note = Synthetic Construct
HSPTTSLTLSYKGGGGSGGGGSGGGGSHSPTTSLTLSYK

SEQ ID NO: 166 - protein, 39 aa, synthetic construct
  REGION 1..39: note = Synthetic Construct
SYPSNALSLHKYGGGGSGGGGSGGGGSSYPSNALSLHKY

SEQ ID NO: 167 - protein, 39 aa, synthetic construct
  REGION 1..39: note = Synthetic Construct
TLGLRPVPVATTGGGGSGGGGSGGGGSTLGLRPVPVATT

SEQ ID NO: 168 - protein, 39 aa, synthetic construct
  REGION 1..39: note = Synthetic Construct
HSPTTSLTLSYKGGGGSGGGGSGGGGSSYPSNALSLHKY

SEQ ID NO: 169 - protein, 39 aa, synthetic construct
  REGION 1..39: note = Synthetic Construct
HSPTTSLTLSYKGGGGSGGGGSGGGGSTLGLRPVPVATT

SEQ ID NO: 170 - protein, 39 aa, synthetic construct
  REGION 1..39: note = Synthetic Construct
SYPSNALSLHKYGGGGSGGGGSGGGGSHSPTTSLTLSYK

SEQ ID NO: 171 - protein, 39 aa, synthetic construct
  REGION 1..39: note = Synthetic Construct
TLGLRPVPVATTGGGGSGGGGSGGGGSHSPTTSLTLSYK

SEQ ID NO: 172 - protein, 39 aa, synthetic construct
  REGION 1..39: note = Synthetic Construct
SYPSNALSLHKYGGGGSGGGGSGGGGSTLGLRPVPVATT

SEQ ID NO: 173 - protein, 39 aa, synthetic construct
  REGION 1..39: note = Synthetic Construct
TLGLRPVPVATTGGGGSGGGGSGGGGSSYPSNALSLHKY

SEQ ID NO: 174 - protein, 66 aa, synthetic construct
  REGION 1..66: note = Synthetic Construct
HSPTTSLTLSYKGGGGSGGGGSGGGGSHSPTTSLTLSYKGGGGSGGGGSGGGGSHSPTTSLTLSYK

SEQ ID NO: 175 - protein, 66 aa, synthetic construct
  REGION 1..66: note = Synthetic Construct
SYPSNALSLHKYGGGGSGGGGSGGGGSSYPSNALSLHKYGGGGSGGGGSGGGGSSYPSNALSLHKY

SEQ ID NO: 176 - protein, 66 aa, synthetic construct
  REGION 1..66: note = Synthetic Construct
TLGLRPVPVATTGGGGSGGGGSGGGGSTLGLRPVPVATTGGGGSGGGGSGGGGSTLGLRPVPVATT

SEQ ID NO: 177 - protein, 66 aa, synthetic construct
  REGION 1..66: note = Synthetic Construct
HSPTTSLTLSYKGGGGSGGGGSGGGGSSYPSNALSLHKYGGGGSGGGGSGGGGSTLGLRPVPVATT

SEQ ID NO: 178 - protein, 65 aa, synthetic construct
  REGION 1..65: note = Synthetic Construct
SYPSNALSLHKYGGGGSGGGGSGGGGTLGLRPVPVATTGGGGSGGGGSGGGGSHSPTTSLTLSYK

SEQ ID NO: 179 - protein, 66 aa, synthetic construct
  REGION 1..66: note = Synthetic Construct
TLGLRPVPVATTGGGGSGGGGSGGGGSSYPSNALSLHKYGGGGSGGGGSGGGGSHSPTTSLTLSYK

SEQ ID NO: 180 - protein, 66 aa, synthetic construct
  REGION 1..66: note = Synthetic Construct
HSPTTSLTLSYKGGGGSGGGGSGGGGSTLGLRPVPVATTGGGGSGGGGSGGGGSSYPSNALSLHKY

(19) United States
(12) Patent Application Publication
(10) Pub. No.: US 2024/0325551 A1
(43) Pub. Date: Oct. 3, 2024

(54) METASTATIC BRAIN TUMOR TARGETING PEPTIDES

(71) Applicant: H. Lee Moffitt Cancer Center and Research Institute, Inc., Tampa, FL (US)

(72) Inventors: James K. Liu, Lutz, FL (US); Daniel Abate-Daga, Tampa, FL (US); Cecilia Ramello, Tampa, FL (US)

(21) Appl. No.: 18/733,364

(22) Filed: Jun. 4, 2024

Related U.S. Application Data
(62) Division of application No. 17/282,257, filed on Apr. 1, 2021.
(60) Provisional application No. 62/746,850, filed on Oct. 17, 2018.
(60) Provisional application No. 62/755,434, filed on Nov. 3, 2018.

(51) Int. Cl. A61K 47/64; A61K 35/17; A61K 39/395; A61K 47/65; C07K 14/725

(52) U.S. Cl. CPC A61K 47/64; A61K 35/17; A61K 39/3955; A61K 47/65; C07K 14/7051; C07K 2319/02; C07K 2319/03

(57) ABSTRACT

Compositions and methods are disclosed for targeted treatment of lung cancer-derived metastatic brain tumors. In particular, tumor targeting agents comprising targeting peptides are disclosed that can target a payload, such as a therapeutic or diagnostic agent, to lung cancer-derived metastatic brain tumors. Chimeric antigen receptor (CAR) polypeptides are disclosed that can be used with adoptive cell transfer to target and kill lung cancer-derived metastatic brain tumors.

### CROSS-REFERENCE TO RELATED APPLICATIONS

This application is a divisional of copending application Ser. No. 17/282,257, filed Apr. 1, 2021, which is a National Stage of International Application No. PCT/US2019/056712, filed Oct. 17, 2019, which claims benefit of U.S. Provisional Application No. 62/746,850, filed Oct. 17, 2018, and U.S. Provisional Application No. 62/755,434, filed Nov. 3, 2018, which are hereby incorporated herein by reference in their entireties.

### STATEMENT REGARDING FEDERALLY SPONSORED RESEARCH OR DEVELOPMENT

This invention was made with Government Support under Grant No. CA193489 awarded by the National Institutes of Health. The Government has certain rights in the invention.

### SEQUENCE LISTING

This application contains a sequence listing filed in ST.26 format entitled “320803-1811 Sequence Listing” created on Jun. 4, 2024, having 226,381 bytes. The content of the sequence listing is incorporated herein in its entirety.

### BACKGROUND

Brain metastases are the most common intracranial tumors in adults, with 150,000 to 170,000 cases annually in the United States as of 2004. Sources of cerebral metastasis may be cancers of the lung, breast, skin, colon, or kidney, with lung cancer being the most common. The therapeutic paradigms for these tumors include neurosurgical intervention, whole-brain and focused radiation modalities, chemotherapy, and immunotherapy.

### SUMMARY

Compositions and methods are disclosed for targeted treatment of lung cancer-derived metastatic brain tumors. In particular, peptides were identified that selectively bind lung cancer-derived metastatic brain tumors. Therefore, tumor targeting agents are disclosed that comprise these targeting peptides, which can be used to target a payload, such as a therapeutic or diagnostic agent, to lung cancer-derived metastatic brain tumors. The disclosed tumor targeting agent can be used to deliver any payload to a lung cancer-derived metastatic brain tumor in a subject.

These peptides can also be used to construct chimeric antigen receptor (CAR) polypeptides for use in CAR-T cells that can be used to treat lung cancer-derived metastatic brain tumors. Therefore, disclosed herein are chimeric antigen receptor (CAR) polypeptides that can be used with adoptive cell transfer to target and kill lung cancer-derived metastatic brain tumors. The disclosed CAR polypeptides contain in an ectodomain a tumor targeting agent disclosed herein that can bind antigens on lung cancer-derived metastatic brain tumors. Also disclosed is an immune effector cell genetically modified to express the disclosed CAR polypeptide. Also disclosed is a method of providing an anti-tumor immunity in a subject with lung cancer-derived metastatic brain tumors that involves administering to the subject an effective amount of an immune effector cell genetically modified with a disclosed CAR.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B illustrate the in vitro (FIG. 1A) and in vivo (FIG. 1B) phage display screenings used to isolate lung cancer derived metastatic brain tumors.

FIGS. 2A to 2C show binding of BRDT (LBM1) peptide applied to Primary Lung Cancer Cells, Non Malignant Brain Cells, Lung Cancer Brain Metastases Cells (MH1002, MH1016, H1915) analyzed through flow cytometry. Flow cytometry demonstrated right shift with BRDT peptide applied to lung cancer BM cells when compared to primary lung cancer cells and non-targeting peptides.

FIG. 3 shows positive binding of BRDT (LBM1) peptide applied to Lung Cancer Brain Metastases cells (MH1002). LBM1 peptide applied to primary lung cancer cells (Primary) and non-malignant brain cells shows no binding.

FIGS. 4A to 4C show binding of LBM2 peptide applied to Primary Lung Cancer Cells, Non Malignant Brain Cells, Lung Cancer Brain Metastases Cells (MH1002, MH1016, H1915) analyzed through flow cytometry. Flow cytometry demonstrated right shift with LBM2 peptide applied to lung cancer BM cells when compared to primary lung cancer cells and non-targeting peptides.

FIG. 5 shows positive binding of LBM2 peptide applied to Lung Cancer Brain Metastases cells (MH1002). LBM2 peptide applied to primary lung cancer cells (Primary) and non-malignant brain cells shows no binding.

FIGS. 6A to 6C show binding AMOT (LBM4) peptide applied to Primary Lung Cancer Cells, Non Malignant Brain Cells, Lung Cancer Brain Metastases Cells (MH1002, MH1016, H1915) analyzed through flow cytometry. Flow cytometry demonstrated right shift with AMOT peptide applied to lung cancer BM cells when compared to primary lung cancer cells and non targeting peptides.

FIG. 7A shows no binding of AMOT (LBM4) peptide applied to primary lung cancer cells and non-malignant brain cells. FIG. 7B shows positive binding of AMOT peptide applied to Lung Cancer Brain Metastases cells (M1002, M1016, H1915).

FIGS. 8A and 8B show peptide signal time course. Mice were 29 days post implantation of intracranial lung cancer brain metastatic tumor. 400 μM of LBM4 peptide was injected via tail vein. Mice were imaged at 0, 3, 6, 12, 24, and 48 hours after initial injection. Graph demonstrate signal height at 3 hours with gradual decrease. NT=non targeting peptide (non specific peptide).

FIG. 9 shows binding of 400 uM peptide injected into the tail vein of mice with intracranial implanted lung cancer brain metastasis. Cy5.5 visualizes peptide binding.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Compositions and methods are disclosed for targeted treatment of lung cancer-derived metastatic brain tumors. In particular, peptides were identified that selectively bind lung cancer-derived metastatic brain tumors.

In some embodiments, the tumor targeting peptide comprises one or more targeting peptides disclosed herein. For example, in some embodiments the targeting peptide comprises the amino acid sequence SYPSNALSLHKY (SEQ ID NO:1, LBM1), or a variant or a fragment having at least 10, 11, 12, 13, 14, or 15 contiguous amino acids and at least 65%, 70%, 75, 80%, 85%, 90%, 95%, or 100% sequence identity to SEQ ID NO:1.

In some embodiments the targeting peptide comprises the amino acid sequence TLGLRPVPVATT (SEQ ID NO:2, LBM2), or a variant or a fragment having at least 10, 11, 12, 13, 14, or 15 contiguous amino acids and at least 65%, 70%, 75, 80%, 85%, 90%, 95%, or 100% sequence identity to SEQ ID NO:2.

In some embodiments the targeting peptide comprises the amino acid sequence YDAIQRPTGQLS (SEQ ID NO:3, LBM3), or a variant or a fragment having at least 10, 11, 12, 13, 14, or 15 contiguous amino acids and at least 65%, 70%, 75, 80%, 85%, 90%, 95%, or 100% sequence identity to SEQ ID NO:3.

In some embodiments the targeting peptide comprises the amino acid sequence HSPTTSLTLSYK (SEQ ID NO:4, LBM4), or a variant or a fragment having at least 10, 11, 12, 13, 14, or 15 contiguous amino acids and at least 65%, 70%, 75, 80%, 85%, 90%, 95%, or 100% sequence identity to SEQ ID NO:4.

In some embodiments the targeting peptide comprises an amino acid sequence selected from the group consisting of ADSNHAYERDSV (SEQ ID NO:5), AESPLTNRGWNP (SEQ ID NO:6), AHPHSDKLVPPR (SEQ ID NO:7), AHSSFGFSHLAV (SEQ ID NO:8), AHVNSSERIHPY (SEQ ID NO:9), ANTELALANRKH (SEQ ID NO:10), APNVGDLTSLLG (SEQ ID NO:11), AQSTTMSTSQRN (SEQ ID NO:12), ARSLEPAPSRHS (SEQ ID NO:13), ATPGSFQTIRAN (SEQ ID NO:14), AMNMRSLTYSDL (SEQ ID NO:15), APQTSDSTLTVM (SEQ ID NO:16), CVTPSNRDCSHS (SEQ ID NO:17), EALLKPYSFAYP (SEQ ID NO:18), EHVDMWNLVSAS (SEQ ID NO:19), EKYPPSSMDQRS (SEQ ID NO:20), ELVSSSDQRNKN (SEQ ID NO:21), FPKSVYPAMPRP (SEQ ID NO:22), FSPTQANTIHRW (SEQ ID NO:23), FTNPFGPYPTGR (SEQ ID NO:24), GKQTLHSFATHW (SEQ ID NO:25), GPWLGSNMRGAS (SEQ ID NO:26), GRDMPMSALMRH (SEQ ID NO:27), GSAARTISPSLL (SEQ ID NO:28), GSMFYLPMPERG (SEQ ID NO:29), GTDIIHPRVIFN (SEQ ID NO:30), GTASRTHSYYSL (SEQ ID NO:31), HNTPMLDSRGNN (SEQ ID NO:32), HPALSQHLGPVA (SEQ ID NO:33), HTPHPGGRSVPP (SEQ ID NO:34), HYKPHVSSLPLA (SEQ ID NO:35), IGSKSPLRLTMD (SEQ ID NO:36), KLTTDKVRTITL (SEQ ID NO:37), LIAGWDISSKR (SEQ ID NO:38), LPKVSVPRHPSV (SEQ ID NO:39), LTPHLGTHKSTT (SEQ ID NO:40), MKAHHSQLYPRH (SEQ ID NO:41), MNIAELRNSDLN (SEQ ID NO:42), NANHNPLNLKSA (SEQ ID NO:43), QFAKTSDPGSLT (SEQ ID NO:44), QIFNHSPNDPKK (SEQ ID NO:45), QYVPYLPPAIL (SEQ ID NO:46), NNTDHRQLTSTT (SEQ ID NO:47), NYLPHQSSSPSRG (SEQ ID NO:48), QYVPYLPPAIL (SEQ ID NO:49), RIPMPSYMNHM (SEQ ID NO:50), SDASGIPKRLAFP (SEQ ID NO:51), SDTTSKHLYTRL (SEQ ID NO:52), SGYSAEGGKPVL (SEQ ID NO:53), SGYSQGGKPVL (SEQ ID NO:54), SHGISSTPPGQA (SEQ ID NO:55), SLPLAIHNSRPN (SEQ ID NO:56), SLTDYVRKGPRI (SEQ ID NO:57), SNPSAHKDDSKR (SEQ ID NO:58), SRYMGPLDLLAP (SEQ ID NO:59), SRYMGPLDLLAP (SEQ ID NO:60), SSMPINSPATRQ (SEQ ID NO:61), SVDYSFSNRTDR (SEQ ID NO:62), SYHLSNTFRVQA (SEQ ID NO:63), TAVLAPQPWLNL (SEQ ID NO:64), TGSAKFLQRDTH (SEQ ID NO:65), TKPSVVHWPMIR (SEQ ID NO:66), TPHGYQPMQGKT (SEQ ID NO:67), TQGSGFSSPILP (SEQ ID NO:68), TTDFFYKRTFFT (SEQ ID NO:69), TTRPNHVHLAKI (SEQ ID NO:70), VAQRHTLTSITV (SEQ ID NO:71), VDAKYGRHIPPV (SEQ ID NO:72), VPTQWTHRESHA (SEQ ID NO:73), WGVTKPIRTSTL (SEQ ID NO:74), WGVTKPIRTSTL (SEQ ID NO:75), WYPSNALSLHK (SEQ ID NO:76), YASQSALSHSAR (SEQ ID NO:77), and YVKSHTTTAVRQ (SEQ ID NO:78), or a variant or a fragment having at least 10, 11, 12, 13, 14, or 15 contiguous amino acids and at least 65%, 70%, 75, 80%, 85%, 90%, 95%, or 100% sequence identity to any one of SEQ ID NOs:5-78.

Tumor targeting agents are disclosed that comprise the disclosed targeting peptides, which can be used to target a payload, such as a therapeutic or diagnostic agent, to lung cancer-derived metastatic brain tumors.

In some embodiments, the molecule is defined by the formula:

NH₃-ACA-TTP-COOH, or

NH₃-TTP-ACA-COOH,
wherein “TTP” represents the tumor targeting peptide,
wherein “ACA” represents the anti-cancer agent, and
wherein “-” represents a bivalent linker.

In some embodiments, the tumor targeting agent comprises 1, 2, 3, 4, 5, 6, 7, 8, 9, or 10 targeting peptides disclosed herein, optionally separated by a linker. The targeting peptide can be a repeat of the same peptide, a mixture of different peptides, or a combination thereof. This oligomer can be linear or branched using compositions and methods known in the art. The tumor targeting agent can be comprised of natural or synthetic amino acids.

Suitable linkers for oligomeric peptides are known in the art. In some embodiments, the linker comprises the amino acid sequence GGGS (SEQ ID NO:79), GGGSGG (SEQ ID NO:80), GGGSGGGS (SEQ ID NO:81), GGGGSGGGGSGGGGS (SEQ ID NO:82), GGGGSGGGGSGGGGSGG (SEQ ID NO:83), or GGGGSGGGGSGGGGSGGGGS (SEQ ID NO:84), or GSTSGSGKPGSGEGSTKG (SEQ ID NO:164).

Therefore, in some embodiments, the targeting agent comprises the amino acid sequence: HSPTTSLTLSYKGGGGSGGGGSGGGGSHSPTTSLTLSYK (LBM4-LBM4, SEQ ID NO:165). In some embodiments, the targeting agent comprises the amino acid sequence: SYPSNALSLHKYGGGGSGGGGSGGGGSSYPSNALSLHKY (LBM1-LBM1, SEQ ID NO:166). In some embodiments, the targeting agent comprises the amino acid sequence: TLGLRPVPVATTGGGGSGGGGSGGGGSTLGLRPVPVATT (LBM2-LBM2, SEQ ID NO:167).

In some embodiments, the targeting agent comprises the amino acid sequence: HSPTTSLTLSYKGGGGSGGGGSGGGGSSYPSNALSLHKY (LBM4-LBM1, SEQ ID NO:168). In some embodiments, the targeting agent comprises the amino acid sequence: HSPTTSLTLSYKGGGGSGGGGSGGGGSTLGLRPVPVATT (LBM4-LBM2, SEQ ID NO:169). In some embodiments, the targeting agent comprises the amino acid sequence: SYPSNALSLHKYGGGGSGGGGSGGGGSHSPTTSLTLSYK (LBM1-LBM4, SEQ ID NO:170). In some embodiments, the targeting agent comprises the amino acid sequence: TLGLRPVPVATTGGGGSGGGGSGGGGSHSPTTSLTLSYK (LBM2-LBM4, SEQ ID NO:171).

In some embodiments, the targeting agent comprises the amino acid sequence: SYPSNALSLHKYGGGGSGGGGSGGGGSTLGLRPVPVATT (LBM1-LBM2, SEQ ID NO:172). In some embodiments, the targeting agent comprises the amino acid sequence: TLGLRPVPVATTGGGGSGGGGSGGGGSSYPSNALSLHKY (LBM2-LBM1, SEQ ID NO:173).

Therefore, in some embodiments, the targeting agent comprises the amino acid sequence: HSPTTSLTLSYKGGGGSGGGGSGGGGSHSPTTSLTLSYKGGGGSGGGGSGGGGSHSPT TSLTLSYK (LBM4-LBM4-LBM4, SEQ ID NO:174). In some embodiments, the targeting agent comprises the amino acid sequence: SYPSNALSLHKYGGGGSGGGGSGGGGSSYPSNALSLHKYGGGGSGGGGSGGGGSSYP SNALSLHKY (LBM1-LBM1-LBM1, SEQ ID NO:175). In some embodiments, the targeting agent comprises the amino acid sequence: TLGLRPVPVATTGGGGSGGGGSGGGGSTLGLRPVPVATTGGGGSGGGGSGGGGSTLGL RPVPVATT (LBM2-LBM2-LBM2, SEQ ID NO:176).

Therefore, in some embodiments, the targeting agent comprises the amino acid sequence: HSPTTSLTLSYKGGGGSGGGGSGGGGSSYPSNALSLHKYGGGGSGGGGSGGGGSTLGL RPVPVATT (LBM4-LBM1-LBM2, SEQ ID NO:177). In some embodiments, the targeting agent comprises the amino acid sequence: SYPSNALSLHKYGGGGSGGGGSGGGGTLGLRPVPVATTGGGGSGGGGSGGGGSHSPTT SLTLSYK (LBM1-LBM2-LBM4, SEQ ID NO:178). In some embodiments, the targeting agent comprises the amino acid sequence: TLGLRPVPVATTGGGGSGGGGSGGGGSSYPSNALSLHKYGGGGSGGGGSGGGGSHSPT TSLTLSYK (LBM2-LBM1-LBM4, SEQ ID NO:179). In some embodiments, the targeting agent comprises the amino acid sequence: HSPTTSLTLSYKGGGGSGGGGSGGGGSTLGLRPVPVATTGGGGSGGGGSGGGGSSYPS NALSLHKY (LBM2-LBM2-LBM1, SEQ ID NO:180).

The disclosed tumor targeting agent can be used to deliver any payload to a lung cancer-derived metastatic brain tumor in a subject. The payload can be a therapeutic or diagnostic agent. In some embodiments, the payload is an anti-cancer agent that can cause apoptosis or pyroptosis of the targeted tumor cell. In some embodiments, the anti-cancer agent is a small molecule drug. The anti-cancer agent can be a chemotherapy agent, such as drugs that stop DNA building block synthesis (e.g., methotrexate, fluorouracil, hydroxyurea, lurtotecan, mercaptopurine, pentostatin and pirarubicin), drugs that directly damage DNA (e.g., cisplatin, daunorubicin, doxorubicin, etoposide, teniposide, camptothecin, topotecan, irinotecan, rubitecan, belotecan), drugs that affect mitotic spindle synthesis or breakdown (e.g., vinblastine, vincristine, vinorelbine, vinflunine, vindesine, docetaxel, larotaxel, ortataxel, paclitaxel, tesetaxel, ixabepilone and epithilones), or drugs that disrupt angiogenesis (e.g., anti-VEGF antibody, angiostatin, endostatin, and tumstatin). Alternatively, the anti-cancer agent can be a radiotherapy agent (e.g., 90Y, 1251, 188Re, 111In DTPA, or 1311 Sodium iodide).

Examples of anti-cancer drugs or antineoplastics to be attached to the tumor targeting peptides described herein include, but are not limited to, aclarubicin, altretamine, aminopterin, amrubicin, azacitidine, azathioprine, belotecan, busulfan, camptothecin, capecitabine, carboplatin, carmofur, carmustine, chlorambucil, cisplatin, cladribine, clofarabine, cyclophosphamide, cytarabine, daunorubicin, decitabine, doxorubicin, epirubicin, etoposide, floxuridine, fludarabine, 5-fluorouracil, fluorouracil, gemcitabine, idarubicin, ifosfamide, irinotecan, mechlorethamine, melphalan, mercaptopurine, methotrexate, mitoxantrone, nedaplatin, oxaliplatin, paclitaxel, pemetrexed, pentostatin, pirarubicin, pixantrone, procarbazine, pyrimethamine raltitrexed, rubitecan, satraplatin, streptozocin, thioguanine, triplatin tetranitrate, teniposide, topotecan, tegafur, trimethoprim, uramustine, valrubicin, vinblastine, vincristine, vindesine, vinflunine, vinorelbine, and zorubicin.

In come embodiments, the tumor targeting peptide is linked to a vehicle carrier, which is associated with the anti-cancer agent. In one example, the vehicle carrier encapsulates the anti-cancer agent. Vehicle carriers include, but are not limited to, micelle, liposome (e.g., cationic liposome), nanoparticle, microsphere, or biodegradable polymer. A tumor targeting peptide can be tethered to a vehicle carrier by a variety of linkages (e.g., a disulfide linkage, an acid labile linkage, a peptide-based linkage, an oxyamino linkage, or a hydrazine linkage). To improve the association between the peptide and the vehicle carrier, the peptide can be modified by a suitable polymer, such as PEG (peglyated). The detectable label or the anti-cancer agent can be encapsulated within the vehicle via, e.g., association with lipophilic molecules, which can aid in the delivery of the detectable label or the anti-cancer agent to the interior of the vehicle.

In some embodiments, a tumor targeting peptide described herein is linked to a liposome (as a vehicle carrier) that encapsulates one or more agents of interest (e.g., an anti-cancer agent). Liposome is a vesicle comprised of one or more concentrically ordered lipid bilayers, which encapsulate an aqueous phase. The aqueous phase typically contains an agent to be delivered to a target site such as a tumor site. Upon reaching the target site, the liposome fuses with the plasma membranes of local cells to release the agent into the cytosol. Alternatively, the liposome is endocytosed or otherwise taken in by the cells as the content of a transport vesicle (e.g., an endosome or phagosome). Once in the transport vesicle, the liposome either degrades or fuses with the membrane of the vesicle and releases its contents. Liposome membranes can be constructed so that they become destabilized when the nearby environment becomes acidic (see, e.g., PNAS 84:7851, 1987; Biochemistry 28:908, 1989). Thus, when liposomes enter a target cell, they become destabilized to release their encapsulated contents. This destabilization process is termed fusogenesis. Dioleoylphosphatidylethanolamine (DOPE) is commonly used to facilitate this process.

A variety of methods are available for preparing liposomes. See, e.g., Szoka et al., Ann. Rev. Biophys. Bioeng. 9:467 (1980), U.S. Pat. Nos. 4,186,183, 4,217,344, 4,235,871, 4,261,975, 4,485,054, 4,501,728, 4,774,085, 4,837,028, 4,235,871, 4,261,975, 4,485,054, 4,501,728, 4,774,085, 4,837,028, 4,946,787, PCT Publication No. WO 91/17424, Deamer & Bangham, Biochim. Biophys. Acta 443:629-634 (1976); Fraley, et al., PNAS 76:3348-3352 (1979); Hope et al., Biochim. Biophys. Acta 812:55-65 (1985); Mayer et al., Biochim. Biophys. Acta 858:161-168 (1986); Williams et al., PNAS 85:242-246 (1988); Liposomes (Ostro (ed.), 1983, Chapter 1); Hope et al., Chem. Phys. Lip. 40:89 (1986); Gregoriadis, Liposome Technology (1984) and Lasic, Liposomes: from Physics to Applications (1993)). Suitable methods include, for example, sonication, extrusion, high pressure/homogenization, microfluidization, detergent dialysis, calcium-induced fusion of small liposome vehicles and ether fusion methods, all of which are well known in the art.

In come embodiments, the tumor targeting peptide can be linked directly to an anti-cancer agent via methods known in the art to form a peptide-drug conjugate (PDC). In some embodiments, the tumor targeting peptide is linked to the anti-cancer agent by a linker. This linker can be designed to bear an enzyme-hydrolyzable unit (EHU) like a carboxylic ester or an amide bond, cleaved by esterases and amidases, respectively. The most commonly utilized linkers that bear a carboxylic ester bond, as the enzyme-hydrolyzable unit, are succinyl (derived from succinic acid) and glutaryl (derived from glutaric acid). Concerning the utilization of amide bond in the linker as the unit tethering the drug and the peptide, it can be tailored to be cleaved based on the targeted tissue and/or type of cancer where a specific protease is statistically upregulated (e.g. cathepsin B upregulated in various malignancies including lung, brain, prostate and breast). Another class of linkers is the stimuli-responsive/degradable linkers, designed to achieve an efficient release of the drug from the bioconjugate in the tumor microenvironment. Such linkers are rationally designed to be cleaved when they sense specific stimuli in the environment of cancerous cells (slightly acidic pH, enhanced levels of reducing agents and/or enzymes) or external stimuli (ultrasound, temperature, irradiation). Specifically, there are certain bonds like imine, oxime, hydrazone, orthoester, acetal, vinyl ether and polyketal that are known to undergo hydrolysis at acidic pH, while being extremely stable during blood circulation. Therefore, acid-labile bonds could be hydrolyzed in the slightly acidic microenvironment and/or in the acidic cellular compartments of cancer cells and consequently release the active drug. Additionally, disulfide linkers are often adopted in PDCs, since they are cleaved by reducing agents like cysteine and glutathione, present in high concentrations in malignant cells. Linkers bearing enzyme-hydrolyzable units (EHU) responsive to proteases are degradable peptide linkers that have attracted significant interest due to the specificity of certain enzymes and there has been a dramatic escalation over in the past years. The most representative examples in this field are the MMP-2/9 (matrix metalloproteinases) and cathepsin B peptide substrates. MMP-2/9 and cathepsin B are proteolytic enzymes present at elevated levels in cancer cells known to participate in human tumor invasion and metastasis. Another rapidly emerging category in PDC linkers that has gained much attention in the last years are the self-immolative or self-destructive spacers/linkers. This type of linkers/spacers offers the capability to release the active drug after simultaneous cascade reactions. Para-amino benzyl alcohol (PABC) is a representative example that can be connected in the amino group via an amide bond to an enzyme-hydrolyzable unit (EHU) and to a tumor-targeting element.

In some embodiments, the payload is a cytotoxic peptide. Cytotoxic peptides are known in the art, and are disclosed for example in U.S. Pat. Nos. 9,998,842, 9,260,478, WO2015/154029, and WO2012/091564, which are all hereby incorporated reference for the teaching of these peptides and their uses.

In some embodiments, the cytotoxic agent comprises a functional nucleic acid that is cytotoxic to cancer cells. For example, the functional nucleic acid can inhibit anti-apoptotic gene targets, e.g., anti-apoptotic Bcl-2 member proteins. The functional nucleic acid can also inhibit targets causing drug sensitization (e.g., PP2A and CDC25c). In some cases, the cytotoxic agent comprises a functional nucleic acid that promotes apoptotic gene targets, e.g., apoptotic Bcl-2 member proteins.

Also disclosed is a pharmaceutical composition comprising the tumor targeting peptide and payload disclosed herein in a pharmaceutically acceptable carrier. Also disclosed is a method for treating a lung cancer-derived metastatic brain tumor in a subject that involves administering to the subject a therapeutically effective amount of a disclosed pharmaceutical composition.

Also disclosed herein are chimeric antigen receptors (CAR) that can specifically recognize lung cancer-derived metastatic brain tumors. Also disclosed are immune effector cells, such as T cells or Natural Killer (NK) cells, that are engineered to express these CARs. Therefore, also disclosed are methods for providing an anti-tumor immunity in a subject with a lung cancer-derived metastatic brain tumor that involves adoptive transfer of the disclosed immune effector cells engineered to express the disclosed CARs.

The disclosed CAR is generally made up of three domains: an ectodomain, a transmembrane domain, and an endodomain. The ectodomain comprises the GSC-binding region and is responsible for antigen recognition. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In some embodiments, the endodomain contains an SD or a CSR, but not both. In these embodiments, an immune effector cell containing the disclosed CAR is only activated if another CAR (or a T-cell receptor) containing the missing domain also binds its respective antigen.

In some embodiments, the CAR polypeptide contains an incomplete endodomain. For example, the CAR polypeptide can contain only an intracellular signaling domain or a co-stimulatory domain, but not both. In these embodiments, the immune effector cell is not activated unless it and a second CAR polypeptide (or endogenous T-cell receptor) that contains the missing domain both bind their respective antigens. Therefore, in some embodiments, the CAR polypeptide contains a CD3 zeta (CD3ζ) signaling domain but does not contain a costimulatory signaling region (CSR). In other embodiments, the CAR polypeptide contains the cytoplasmic domain of CD28, 4-1BB, or a combination thereof, but does not contain a CD3 zeta (CD3ζ) signaling domain (SD).

In some embodiments, the disclosed CAR is used in combination with a CAR that specifically binds another antigen. A dual CAR can be engineered such that one extracellular antigen binding domain is connected to the intracellular costimulatory domain and a second, distinct extracellular antigen binding domain is connected to the intracellular stimulatory domain.

In some embodiments, the disclosed CAR is defined by the formula:

SP-TTP-HG-TM-CSR-ISD; or

SP-TTP-HG-TM-ISD-CSR
wherein “SP” represents a signal peptide,
wherein “TTP” represents a tumor targeting peptide,
wherein “HG” represents and optional hinge domain,
wherein “TM” represents a transmembrane domain,
wherein “CSR” represents a co-stimulatory signaling region,
wherein “ISD” represents an intracellular signaling domain, and
wherein “-” represents a bivalent linker.

As above, the tumor targeting agent can comprise 1, 2, 3, 4, 5, 6, 7, 8, 9, or 10 targeting peptides disclosed herein, optionally separated by a linker. The targeting peptide can be a repeat of the same peptide, a mixture of different peptides, or a combination thereof. This oligomer can be linear or branched using compositions and methods known in the art. The tumor targeting agent can be comprised of natural or synthetic amino acids.

Additional CAR constructs are described, for example, in FresnakAD, et al. Engineered T cells: the promise and challenges of cancer immunotherapy. Nat Rev Cancer. 2016 Aug. 23; 16(9):566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or sCAR.

TRUCKs (T cells redirected for universal cytokine killing) co-express a chimeric antigen receptor (CAR) and an antitumor cytokine. Cytokine expression may be constitutive or induced by T cell activation. Targeted by CAR specificity, localized production of pro-inflammatory cytokines recruits endogenous immune cells to tumor sites and may potentiate an antitumor response.

Universal, allogeneic CAR T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

Marked CAR T cells express a CAR plus a tumor epitope to which an existing monoclonal antibody agent binds. In the setting of intolerable adverse effects, administration of the monoclonal antibody clears the CAR T cells and alleviates symptoms with no additional off-tumor effects.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets.

A dual CAR T cell expresses two separate CARs with different ligand binding targets; one CAR includes only the CD3ζ domain and the other CAR includes only the co-stimulatory domain(s). Dual CAR T cell activation requires co-expression of both targets on the tumor.

A safety CAR (sCAR) consists of an extracellular domain fused to an intracellular inhibitory domain. sCAR T cells co-expressing a standard CAR become activated only when encountering target cells that possess the standard CAR target but lack the sCAR target.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIγ, FcγRIIIγ, FcεRIβ (FCERIB), and FcεRIγ (FCERIG).

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD3ζ chain, which is the primary transmitter of signals from endogenous TCRs. Second-generation CARs add intracellular signaling domains from various costimulatory protein receptors (e.g., CD28, 41 BB, ICOS) to the endodomain of the CAR to provide additional signals to the T cell. Preclinical studies have indicated that the second generation of CAR designs improves the antitumor activity of T cells. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., GSC-binding domain) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

Tables 1, 2, and 3 below provide some example combinations of tumor targeting peptide (TTP), co-stimulatory signaling regions, and intracellular signaling domain that can occur in the disclosed CARs.

TABLE 1
First Generation CARs

| | Signal |
|---|---|
| | Domain |
| TTP | CD8 |
| TTP | CD3ζ |
| TTP | CD3δ |
| TTP | CD3γ |
| TTP | CD3ε |
| TTP | FcγRI-γ |
| TTP | FcγRIII-γ |
| TTP | FcεRIβ |
| TTP | FcεRIγ |
| TTP | DAP10 |
| TTP | DAP12 |
| TTP | CD32 |
| TTP | CD79a |

TABLE 2
Second Generation CARs

| | Co-stimulatory | Signal |
|---|---|---|
| | Signal | Domain |
| TTP | CD28 | CD8 |
| TTP | CD28 | CD3ζ |
| TTP | CD28 | CD3δ |
| TTP | CD28 | CD3γ |
| TTP | CD28 | CD3ε |
| TTP | CD28 | FcγRI-γ |
| TTP | CD28 | FcγRIII-γ |
| TTP | CD28 | FcεRIβ |
| TTP | CD28 | FcεRIγ |
| TTP | CD28 | DAP10 |
| TTP | CD28 | DAP12 |
| TTP | CD28 | CD32 |
| TTP | CD28 | CD79a |
| TTP | CD28 | CD79b |
| TTP | CD8 | CD8 |
| TTP | CD8 | CD3ζ |
| TTP | CD8 | CD3δ |
| TTP | CD8 | CD3γ |
| TTP | CD8 | CD3ε |
| TTP | CD8 | FcγRI-γ |
| TTP | CD8 | FcγRIII-γ |
| TTP | CD8 | FcεRIβ |
| TTP | CD8 | FcεRIγ |
| TTP | CD8 | DAP10 |
| TTP | CD8 | DAP12 |
| TTP | CD8 | CD32 |
| TTP | CD8 | CD79a |
| TTP | CD8 | CD79b |
| TTP | CD4 | CD8 |
| TTP | CD4 | CD3ζ |
| TTP | CD4 | CD3δ |
| TTP | CD4 | CD3γ |
| TTP | CD4 | CD3ε |
| TTP | CD4 | FcγRI-γ |
| TTP | CD4 | FcγRIII-γ |
| TTP | CD4 | FcεRIβ |
| TTP | CD4 | FcεRIγ |
| TTP | CD4 | DAP10 |
| TTP | CD4 | DAP12 |
| TTP | CD4 | CD32 |
| TTP | CD4 | CD79a |
| TTP | CD4 | CD79b |
| TTP | b2c | CD8 |
| TTP | b2c | CD3ζ |
| TTP | b2c | CD3δ |
| TTP | b2c | CD3γ |
| TTP | b2c | CD3ε |
| TTP | b2c | FcγRI-γ |
| TTP | b2c | FcγRIII-γ |
| TTP | b2c | FcεRIβ |
| TTP | b2c | FcεRIγ |
| TTP | b2c | DAP10 |
| TTP | b2c | DAP12 |
| TTP | b2c | CD32 |
| TTP | b2c | CD79a |
| TTP | b2c | CD79b |
| TTP | CD137/41BB | CD8 |
| TTP | CD137/41BB | CD3ζ |
| TTP | CD137/41BB | CD3δ |
| TTP | CD137/41BB | CD3γ |
| TTP | CD137/41BB | CD3ε |
| TTP | CD137/41BB | FcγRI-γ |
| TTP | CD137/41BB | FcγRIII-γ |
| TTP | CD137/41BB | FcεRIβ |
| TTP | CD137/41BB | FcεRIγ |
| TTP | CD137/41BB | DAP10 |
| TTP | CD137/41BB | DAP12 |
| TTP | CD137/41BB | CD32 |
| TTP | CD137/41BB | CD79a |
| TTP | CD137/41BB | CD79b |
| TTP | ICOS | CD8 |
| TTP | ICOS | CD3ζ |
| TTP | ICOS | CD3δ |
| TTP | ICOS | CD3γ |
| TTP | ICOS | CD3ε |
| TTP | ICOS | FcγRI-γ |
| TTP | ICOS | FcγRIII-γ |
| TTP | ICOS | FcεRIβ |
| TTP | ICOS | FcεRIγ |
| TTP | ICOS | DAP10 |
| TTP | ICOS | DAP12 |
| TTP | ICOS | CD32 |
| TTP | ICOS | CD79a |
| TTP | ICOS | CD79b |
| TTP | CD27 | CD8 |
| TTP | CD27 | CD3ζ |
| TTP | CD27 | CD3δ |
| TTP | CD27 | CD3γ |
| TTP | CD27 | CD3ε |
| TTP | CD27 | FcγRI-γ |
| TTP | CD27 | FcγRIII-γ |
| TTP | CD27 | FcεRIβ |
| TTP | CD27 | FcεRIγ |
| TTP | CD27 | DAP10 |
| TTP | CD27 | DAP12 |
| TTP | CD27 | CD32 |
| TTP | CD27 | CD79a |
| TTP | CD27 | CD79b |
| TTP | CD28δ | CD8 |
| TTP | CD28δ | CD3ζ |
| TTP | CD28δ | CD3δ |
| TTP | CD28δ | CD3γ |
| TTP | CD28δ | CD3ε |
| TTP | CD28δ | FcγRI-γ |
| TTP | CD28δ | FcγRIII-γ |
| TTP | CD28δ | FcεRIβ |
| TTP | CD28δ | FcεRIγ |
| TTP | CD28δ | DAP10 |
| TTP | CD28δ | DAP12 |
| TTP | CD28δ | CD32 |
| TTP | CD28δ | CD79a |
| TTP | CD28δ | CD79b |
| TTP | CD80 | CD8 |
| TTP | CD80 | CD3ζ |
| TTP | CD80 | CD3δ |
| TTP | CD80 | CD3γ |
| TTP | CD80 | CD3ε |
| TTP | CD80 | FcγRI-γ |
| TTP | CD80 | FcγRIII-γ |
| TTP | CD80 | FcεRIβ |
| TTP | CD80 | FcεRIγ |
| TTP | CD80 | DAP10 |
| TTP | CD80 | DAP12 |
| TTP | CD80 | CD32 |
| TTP | CD80 | CD79a |
| TTP | CD80 | CD79b |
| TTP | CD86 | CD8 |
| TTP | CD86 | CD3ζ |
| TTP | CD86 | CD3δ |
| TTP | CD86 | CD3γ |
| TTP | CD86 | CD3ε |
| TTP | CD86 | FcγRI-γ |
| TTP | CD86 | FcγRIII-γ |
| TTP | CD86 | FcεRIβ |
| TTP | CD86 | FcεRIγ |
| TTP | CD86 | DAP10 |
| TTP | CD86 | DAP12 |
| TTP | CD86 | CD32 |
| TTP | CD86 | CD79a |
| TTP | CD86 | CD79b |
| TTP | OX40 | CD8 |
| TTP | OX40 | CD3ζ |
| TTP | OX40 | CD3δ |
| TTP | OX40 | CD3γ |
| TTP | OX40 | CD3ε |
| TTP | OX40 | FcγRI-γ |
| TTP | OX40 | FcγRIII-γ |
| TTP | OX40 | FcεRIβ |
| TTP | OX40 | FcεRIγ |
| TTP | OX40 | DAP10 |
| TTP | OX40 | DAP12 |
| TTP | OX40 | CD32 |
| TTP | OX40 | CD79a |
| TTP | OX40 | CD79b |
| TTP | DAP10 | CD8 |
| TTP | DAP10 | CD3ζ |
| TTP | DAP10 | CD3δ |
| TTP | DAP10 | CD3γ |
| TTP | DAP10 | CD3ε |
| TTP | DAP10 | FcγRI-γ |
| TTP | DAP10 | FcγRIII-γ |
| TTP | DAP10 | FcεRIβ |
| TTP | DAP10 | FcεRIγ |
| TTP | DAP10 | DAP10 |
| TTP | DAP10 | DAP12 |
| TTP | DAP10 | CD32 |
| TTP | DAP10 | CD79a |
| TTP | DAP10 | CD79b |
| TTP | DAP12 | CD8 |
| TTP | DAP12 | CD3ζ |
| TTP | DAP12 | CD3δ |
| TTP | DAP12 | CD3γ |
| TTP | DAP12 | CD3ε |
| TTP | DAP12 | FcγRI-γ |
| TTP | DAP12 | FcγRIII-γ |
| TTP | DAP12 | FcεRIβ |
| TTP | DAP12 | FcεRIγ |
| TTP | DAP12 | DAP10 |
| TTP | DAP12 | DAP12 |
| TTP | DAP12 | CD32 |
| TTP | DAP12 | CD79a |
| TTP | DAP12 | CD79b |
| TTP | MyD88 | CD8 |
| TTP | MyD88 | CD3ζ |
| TTP | MyD88 | CD3δ |
| TTP | MyD88 | CD3γ |
| TTP | MyD88 | CD3ε |
| TTP | MyD88 | FcγRI-γ |
| TTP | MyD88 | FcγRIII-γ |
| TTP | MyD88 | FcεRIβ |
| TTP | MyD88 | FcεRIγ |
| TTP | MyD88 | DAP10 |
| TTP | MyD88 | DAP12 |
| TTP | MyD88 | CD32 |
| TTP | MyD88 | CD79a |
| TTP | MyD88 | CD79b |
| TTP | CD7 | CD8 |
| TTP | CD7 | CD3ζ |
| TTP | CD7 | CD3δ |
| TTP | CD7 | CD3γ |
| TTP | CD7 | CD3ε |
| TTP | CD7 | FcγRI-γ |
| TTP | CD7 | FcγRIII-γ |
| TTP | CD7 | FcεRIβ |
| TTP | CD7 | FcεRIγ |
| TTP | CD7 | DAP10 |
| TTP | CD7 | DAP12 |
| TTP | CD7 | CD32 |
| TTP | CD7 | CD79a |
| TTP | CD7 | CD79b |
| TTP | BTNL3 | CD8 |
| TTP | BTNL3 | CD3ζ |
| TTP | BTNL3 | CD3δ |
| TTP | BTNL3 | CD3γ |
| TTP | BTNL3 | CD3ε |
| TTP | BTNL3 | FcγRI-γ |
| TTP | BTNL3 | FcγRIII-γ |
| TTP | BTNL3 | FcεRIβ |
| TTP | BTNL3 | FcεRIγ |
| TTP | BTNL3 | DAP10 |
| TTP | BTNL3 | DAP12 |
| TTP | BTNL3 | CD32 |
| TTP | BTNL3 | CD79a |
| TTP | BTNL3 | CD79b |
| TTP | NKG2D | CD8 |
| TTP | NKG2D | CD3ζ |
| TTP | NKG2D | CD3δ |
| TTP | NKG2D | CD3γ |
| TTP | NKG2D | CD3ε |
| TTP | NKG2D | FcγRI-γ |
| TTP | NKG2D | FcγRIII-γ |
| TTP | NKG2D | FcεRIβ |
| TTP | NKG2D | FcεRIγ |
| TTP | NKG2D | DAP10 |
| TTP | NKG2D | DAP12 |
| TTP | NKG2D | CD32 |
| TTP | NKG2D | CD79a |
| TTP | NKG2D | CD79b |

TABLE 3
Third Generation CARs

| | Co-stimulatory | Co-stimulatory | Signal |
|---|---|---|---|
| | Signal | Signal | Domain |
| TTP | CD28 | CD28 | CD8 |
| TTP | CD28 | CD28 | CD3ζ |
| TTP | CD28 | CD28 | CD3δ |
| TTP | CD28 | CD28 | CD3γ |
| TTP | CD28 | CD28 | CD3ε |
| TTP | CD28 | CD28 | FcγRI-γ |
| TTP | CD28 | CD28 | FcγRIII-γ |
| TTP | CD28 | CD28 | FcεRIβ |
| TTP | CD28 | CD28 | FcεRIγ |
| TTP | CD28 | CD28 | DAP10 |
| TTP | CD28 | CD28 | DAP12 |
| TTP | CD28 | CD28 | CD32 |
| TTP | CD28 | CD28 | CD79a |
| TTP | CD28 | CD28 | CD79b |
| TTP | CD28 | CD8 | CD8 |
| TTP | CD28 | CD8 | CD3ζ |
| TTP | CD28 | CD8 | CD3δ |
| TTP | CD28 | CD8 | CD3γ |
| TTP | CD28 | CD8 | CD3ε |
| TTP | CD28 | CD8 | FcγRI-γ |
| TTP | CD28 | CD8 | FcγRIII-γ |
| TTP | CD28 | CD8 | FcεRIβ |
| TTP | CD28 | CD8 | FcεRIγ |
| TTP | CD28 | CD8 | DAP10 |
| TTP | CD28 | CD8 | DAP12 |
| TTP | CD28 | CD8 | CD32 |
| TTP | CD28 | CD8 | CD79a |
| TTP | CD28 | CD8 | CD79b |
| TTP | CD28 | CD4 | CD8 |
| TTP | CD28 | CD4 | CD3ζ |
| TTP | CD28 | CD4 | CD3δ |
| TTP | CD28 | CD4 | CD3γ |
| TTP | CD28 | CD4 | CD3ε |
| TTP | CD28 | CD4 | FcγRI-γ |
| TTP | CD28 | CD4 | FcγRIII-γ |
| TTP | CD28 | CD4 | FcεRIβ |
| TTP | CD28 | CD4 | FcεRIγ |
| TTP | CD28 | CD4 | DAP10 |
| TTP | CD28 | CD4 | DAP12 |
| TTP | CD28 | CD4 | CD32 |
| TTP | CD28 | CD4 | CD79a |
| TTP | CD28 | CD4 | CD79b |
| TTP | CD28 | b2c | CD8 |
| TTP | CD28 | b2c | CD3ζ |
| TTP | CD28 | b2c | CD3δ |
| TTP | CD28 | b2c | CD3γ |
| TTP | CD28 | b2c | CD3ε |
| TTP | CD28 | b2c | FcγRI-γ |
| TTP | CD28 | b2c | FcγRIII-γ |
| TTP | CD28 | b2c | FcεRIβ |
| TTP | CD28 | b2c | FcεRIγ |
| TTP | CD28 | b2c | DAP10 |
| TTP | CD28 | b2c | DAP12 |
| TTP | CD28 | b2c | CD32 |
| TTP | CD28 | b2c | CD79a |
| TTP | CD28 | b2c | CD79b |
| TTP | CD28 | CD137/41BB | CD8 |
| TTP | CD28 | CD137/41BB | CD3ζ |
| TTP | CD28 | CD137/41BB | CD3δ |
| TTP | CD28 | CD137/41BB | CD3γ |
| TTP | CD28 | CD137/41BB | CD3ε |
| TTP | CD28 | CD137/41BB | FcγRI-γ |
| TTP | CD28 | CD137/41BB | FcγRIII-γ |
| TTP | CD28 | CD137/41BB | FcεRIβ |
| TTP | CD28 | CD137/41BB | FcεRIγ |
| TTP | CD28 | CD137/41BB | DAP10 |
| TTP | CD28 | CD137/41BB | DAP12 |
| TTP | CD28 | CD137/41BB | CD32 |
| TTP | CD28 | CD137/41BB | CD79a |
| TTP | CD28 | CD137/41BB | CD79b |
| TTP | CD28 | ICOS | CD8 |
| TTP | CD28 | ICOS | CD3ζ |
| TTP | CD28 | ICOS | CD3δ |
| TTP | CD28 | ICOS | CD3γ |
| TTP | CD28 | ICOS | CD3ε |
| TTP | CD28 | ICOS | FcγRI-γ |
| TTP | CD28 | ICOS | FcγRIII-γ |
| TTP | CD28 | ICOS | FcεRIβ |
| TTP | CD28 | ICOS | FcεRIγ |
| TTP | CD28 | ICOS | DAP10 |
| TTP | CD28 | ICOS | DAP12 |
| TTP | CD28 | ICOS | CD32 |
| TTP | CD28 | ICOS | CD79a |
| TTP | CD28 | ICOS | CD79b |
| TTP | CD28 | CD27 | CD8 |
| TTP | CD28 | CD27 | CD3ζ |
| TTP | CD28 | CD27 | CD3δ |
| TTP | CD28 | CD27 | CD3γ |
| TTP | CD28 | CD27 | CD3ε |
| TTP | CD28 | CD27 | FcγRI-γ |
| TTP | CD28 | CD27 | FcγRIII-γ |
| TTP | CD28 | CD27 | FcεRIβ |
| TTP | CD28 | CD27 | FcεRIγ |
| TTP | CD28 | CD27 | DAP10 |
| TTP | CD28 | CD27 | DAP12 |
| TTP | CD28 | CD27 | CD32 |
| TTP | CD28 | CD27 | CD79a |
| TTP | CD28 | CD27 | CD79b |
| TTP | CD28 | CD28δ | CD8 |
| TTP | CD28 | CD28δ | CD3ζ |
| TTP | CD28 | CD28δ | CD3δ |
| TTP | CD28 | CD28δ | CD3γ |
| TTP | CD28 | CD28δ | CD3ε |
| TTP | CD28 | CD28δ | FcγRI-γ |
| TTP | CD28 | CD28δ | FcγRIII-γ |
| TTP | CD28 | CD28δ | FcεRIβ |
| TTP | CD28 | CD28δ | FcεRIγ |
| TTP | CD28 | CD28δ | DAP10 |
| TTP | CD28 | CD28δ | DAP12 |
| TTP | CD28 | CD28δ | CD32 |
| TTP | CD28 | CD28δ | CD79a |
| TTP | CD28 | CD28δ | CD79b |
| TTP | CD28 | CD80 | CD8 |
| TTP | CD28 | CD80 | CD3ζ |
| TTP | CD28 | CD80 | CD3δ |
| TTP | CD28 | CD80 | CD3γ |
| TTP | CD28 | CD80 | CD3ε |
| TTP | CD28 | CD80 | FcγRI-γ |
| TTP | CD28 | CD80 | FcγRIII-γ |
| TTP | CD28 | CD80 | FcεRIβ |
| TTP | CD28 | CD80 | FcεRIγ |
| TTP | CD28 | CD80 | DAP10 |
| TTP | CD28 | CD80 | DAP12 |
| TTP | CD28 | CD80 | CD32 |
| TTP | CD28 | CD80 | CD79a |
| TTP | CD28 | CD80 | CD79b |
| TTP | CD28 | CD86 | CD8 |
| TTP | CD28 | CD86 | CD3ζ |
| TTP | CD28 | CD86 | CD3δ |
| TTP | CD28 | CD86 | CD3γ |
| TTP | CD28 | CD86 | CD3ε |
| TTP | CD28 | CD86 | FcγRI-γ |
| TTP | CD28 | CD86 | FcγRIII-γ |
| TTP | CD28 | CD86 | FcεRIβ |
| TTP | CD28 | CD86 | FcεRIγ |
| TTP | CD28 | CD86 | DAP10 |
| TTP | CD28 | CD86 | DAP12 |
| TTP | CD28 | CD86 | CD32 |
| TTP | CD28 | CD86 | CD79a |
| TTP | CD28 | CD86 | CD79b |
| TTP | CD28 | OX40 | CD8 |
| TTP | CD28 | OX40 | CD3ζ |
| TTP | CD28 | OX40 | CD3δ |
| TTP | CD28 | OX40 | CD3γ |
| TTP | CD28 | OX40 | CD3ε |
| TTP | CD28 | OX40 | FcγRI-γ |
| TTP | CD28 | OX40 | FcγRIII-γ |
| TTP | CD28 | OX40 | FcεRIβ |
| TTP | CD28 | OX40 | FcεRIγ |
| TTP | CD28 | OX40 | DAP10 |
| TTP | CD28 | OX40 | DAP12 |
| TTP | CD28 | OX40 | CD32 |
| TTP | CD28 | OX40 | CD79a |
| TTP | CD28 | OX40 | CD79b |
| TTP | CD28 | DAP10 | CD8 |
| TTP | CD28 | DAP10 | CD3ζ |
| TTP | CD28 | DAP10 | CD3δ |
| TTP | CD28 | DAP10 | CD3γ |
| TTP | CD28 | DAP10 | CD3ε |
| TTP | CD28 | DAP10 | FcγRI-γ |
| TTP | CD28 | DAP10 | FcγRIII-γ |
| TTP | CD28 | DAP10 | FcεRIβ |
| TTP | CD28 | DAP10 | FcεRIγ |
| TTP | CD28 | DAP10 | DAP10 |
| TTP | CD28 | DAP10 | DAP12 |
| TTP | CD28 | DAP10 | CD32 |
| TTP | CD28 | DAP10 | CD79a |
| TTP | CD28 | DAP10 | CD79b |
| TTP | CD28 | DAP12 | CD8 |
| TTP | CD28 | DAP12 | CD3ζ |
| TTP | CD28 | DAP12 | CD3δ |
| TTP | CD28 | DAP12 | CD3γ |
| TTP | CD28 | DAP12 | CD3ε |
| TTP | CD28 | DAP12 | FcγRI-γ |
| TTP | CD28 | DAP12 | FcγRIII-γ |
| TTP | CD28 | DAP12 | FcεRIβ |
| TTP | CD28 | DAP12 | FcεRIγ |
| TTP | CD28 | DAP12 | DAP10 |
| TTP | CD28 | DAP12 | DAP12 |
| TTP | CD28 | DAP12 | CD32 |
| TTP | CD28 | DAP12 | CD79a |
| TTP | CD28 | DAP12 | CD79b |
| TTP | CD28 | MyD88 | CD8 |
| TTP | CD28 | MyD88 | CD3ζ |
| TTP | CD28 | MyD88 | CD3δ |
| TTP | CD28 | MyD88 | CD3γ |
| TTP | CD28 | MyD88 | CD3ε |
| TTP | CD28 | MyD88 | FcγRI-γ |
| TTP | CD28 | MyD88 | FcγRIII-γ |
| TTP | CD28 | MyD88 | FcεRIβ |
| TTP | CD28 | MyD88 | FcεRIγ |
| TTP | CD28 | MyD88 | DAP10 |
| TTP | CD28 | MyD88 | DAP12 |
| TTP | CD28 | MyD88 | CD32 |
| TTP | CD28 | MyD88 | CD79a |
| TTP | CD28 | MyD88 | CD79b |
| TTP | CD28 | CD7 | CD8 |
| TTP | CD28 | CD7 | CD3ζ |
| TTP | CD28 | CD7 | CD3δ |
| TTP | CD28 | CD7 | CD3γ |
| TTP | CD28 | CD7 | CD3ε |
| TTP | CD28 | CD7 | FcγRI-γ |
| TTP | CD28 | CD7 | FcγRIII-γ |
| TTP | CD28 | CD7 | FcεRIβ |
| TTP | CD28 | CD7 | FcεRIγ |
| TTP | CD28 | CD7 | DAP10 |
| TTP | CD28 | CD7 | DAP12 |
| TTP | CD28 | CD7 | CD32 |
| TTP | CD28 | CD7 | CD79a |
| TTP | CD28 | CD7 | CD79b |
| TTP | CD28 | BTNL3 | CD8 |
| TTP | CD28 | BTNL3 | CD3ζ |
| TTP | CD28 | BTNL3 | CD3δ |
| TTP | CD28 | BTNL3 | CD3γ |
| TTP | CD28 | BTNL3 | CD3ε |
| TTP | CD28 | BTNL3 | FcγRI-γ |
| TTP | CD28 | BTNL3 | FcγRIII-γ |
| TTP | CD28 | BTNL3 | FcεRIβ |
| TTP | CD28 | BTNL3 | FcεRIγ |
| TTP | CD28 | BTNL3 | DAP10 |
| TTP | CD28 | BTNL3 | DAP12 |
| TTP | CD28 | BTNL3 | CD32 |
| TTP | CD28 | BTNL3 | CD79a |
| TTP | CD28 | BTNL3 | CD79b |
| TTP | CD28 | NKG2D | CD8 |
| TTP | CD28 | NKG2D | CD3ζ |
| TTP | CD28 | NKG2D | CD3δ |
| TTP | CD28 | NKG2D | CD3γ |
| TTP | CD28 | NKG2D | CD3ε |
| TTP | CD28 | NKG2D | FcγRI-γ |
| TTP | CD28 | NKG2D | FcγRIII-γ |
| TTP | CD28 | NKG2D | FcεRIβ |
| TTP | CD28 | NKG2D | FcεRIγ |
| TTP | CD28 | NKG2D | DAP10 |
| TTP | CD28 | NKG2D | DAP12 |
| TTP | CD28 | NKG2D | CD32 |
| TTP | CD28 | NKG2D | CD79a |
| TTP | CD28 | NKG2D | CD79b |
| TTP | CD8 | CD28 | CD8 |
| TTP | CD8 | CD28 | CD3ζ |
| TTP | CD8 | CD28 | CD3δ |
| TTP | CD8 | CD28 | CD3γ |
| TTP | CD8 | CD28 | CD3ε |
| TTP | CD8 | CD28 | FcγRI-γ |
| TTP | CD8 | CD28 | FcγRIII-γ |
| TTP | CD8 | CD28 | FcεRIβ |
| TTP | CD8 | CD28 | FcεRIγ |
| TTP | CD8 | CD28 | DAP10 |
| TTP | CD8 | CD28 | DAP12 |
| TTP | CD8 | CD28 | CD32 |
| TTP | CD8 | CD28 | CD79a |
| TTP | CD8 | CD28 | CD79b |
| TTP | CD8 | CD8 | CD8 |
| TTP | CD8 | CD8 | CD3ζ |
| TTP | CD8 | CD8 | CD3δ |
| TTP | CD8 | CD8 | CD3γ |
| TTP | CD8 | CD8 | CD3ε |
| TTP | CD8 | CD8 | FcγRI-γ |
| TTP | CD8 | CD8 | FcγRIII-γ |
| TTP | CD8 | CD8 | FcεRIβ |
| TTP | CD8 | CD8 | FcεRIγ |
| TTP | CD8 | CD8 | DAP10 |
| TTP | CD8 | CD8 | DAP12 |
| TTP | CD8 | CD8 | CD32 |
| TTP | CD8 | CD8 | CD79a |
| TTP | CD8 | CD8 | CD79b |
| TTP | CD8 | CD4 | CD8 |
| TTP | CD8 | CD4 | CD3ζ |
| TTP | CD8 | CD4 | CD3δ |
| TTP | CD8 | CD4 | CD3γ |
| TTP | CD8 | CD4 | CD3ε |
| TTP | CD8 | CD4 | FcγRI-γ |
| TTP | CD8 | CD4 | FcγRIII-γ |
| TTP | CD8 | CD4 | FcεRIβ |
| TTP | CD8 | CD4 | FcεRIγ |
| TTP | CD8 | CD4 | DAP10 |
| TTP | CD8 | CD4 | DAP12 |
| TTP | CD8 | CD4 | CD32 |
| TTP | CD8 | CD4 | CD79a |
| TTP | CD8 | CD4 | CD79b |
| TTP | CD8 | b2c | CD8 |
| TTP | CD8 | b2c | CD3ζ |
| TTP | CD8 | b2c | CD3δ |
| TTP | CD8 | b2c | CD3γ |
| TTP | CD8 | b2c | CD3ε |
| TTP | CD8 | b2c | FcγRI-γ |
| TTP | CD8 | b2c | FcγRIII-γ |
| TTP | CD8 | b2c | FcεRIβ |
| TTP | CD8 | b2c | FcεRIγ |
| TTP | CD8 | b2c | DAP10 |
| TTP | CD8 | b2c | DAP12 |
| TTP | CD8 | b2c | CD32 |
| TTP | CD8 | b2c | CD79a |
| TTP | CD8 | b2c | CD79b |
| TTP | CD8 | CD137/41BB | CD8 |
| TTP | CD8 | CD137/41BB | CD3ζ |
| TTP | CD8 | CD137/41BB | CD3δ |
| TTP | CD8 | CD137/41BB | CD3γ |
| TTP | CD8 | CD137/41BB | CD3ε |
| TTP | CD8 | CD137/41BB | FcγRI-γ |
| TTP | CD8 | CD137/41BB | FcγRIII-γ |
| TTP | CD8 | CD137/41BB | FcεRIβ |
| TTP | CD8 | CD137/41BB | FcεRIγ |
| TTP | CD8 | CD137/41BB | DAP10 |
| TTP | CD8 | CD137/41BB | DAP12 |
| TTP | CD8 | CD137/41BB | CD32 |
| TTP | CD8 | CD137/41BB | CD79a |
| TTP | CD8 | CD137/41BB | CD79b |
| TTP | CD8 | ICOS | CD8 |
| TTP | CD8 | ICOS | CD3ζ |
| TTP | CD8 | ICOS | CD3δ |
| TTP | CD8 | ICOS | CD3γ |
| TTP | CD8 | ICOS | CD3ε |
| TTP | CD8 | ICOS | FcγRI-γ |
| TTP | CD8 | ICOS | FcγRIII-γ |
| TTP | CD8 | ICOS | FcεRIβ |
| TTP | CD8 | ICOS | FcεRIγ |
| TTP | CD8 | ICOS | DAP10 |
| TTP | CD8 | ICOS | DAP12 |
| TTP | CD8 | ICOS | CD32 |
| TTP | CD8 | ICOS | CD79a |
| TTP | CD8 | ICOS | CD79b |
| TTP | CD8 | CD27 | CD8 |
| TTP | CD8 | CD27 | CD3ζ |
| TTP | CD8 | CD27 | CD3δ |
| TTP | CD8 | CD27 | CD3γ |
| TTP | CD8 | CD27 | CD3ε |
| TTP | CD8 | CD27 | FcγRI-γ |
| TTP | CD8 | CD27 | FcγRIII-γ |
| TTP | CD8 | CD27 | FcεRIβ |
| TTP | CD8 | CD27 | FcεRIγ |
| TTP | CD8 | CD27 | DAP10 |
| TTP | CD8 | CD27 | DAP12 |
| TTP | CD8 | CD27 | CD32 |
| TTP | CD8 | CD27 | CD79a |
| TTP | CD8 | CD27 | CD79b |
| TTP | CD8 | CD28δ | CD8 |
| TTP | CD8 | CD28δ | CD3ζ |
| TTP | CD8 | CD28δ | CD3δ |
| TTP | CD8 | CD28δ | CD3γ |
| TTP | CD8 | CD28δ | CD3ε |
| TTP | CD8 | CD28δ | FcγRI-γ |
| TTP | CD8 | CD28δ | FcγRIII-γ |
| TTP | CD8 | CD28δ | FcεRIβ |
| TTP | CD8 | CD28δ | FcεRIγ |
| TTP | CD8 | CD28δ | DAP10 |
| TTP | CD8 | CD28δ | DAP12 |
| TTP | CD8 | CD28δ | CD32 |
| TTP | CD8 | CD28δ | CD79a |
| TTP | CD8 | CD28δ | CD79b |
| TTP | CD8 | CD80 | CD8 |
| TTP | CD8 | CD80 | CD3ζ |
| TTP | CD8 | CD80 | CD3δ |
| TTP | CD8 | CD80 | CD3γ |
| TTP | CD8 | CD80 | CD3ε |
| TTP | CD8 | CD80 | FcγRI-γ |
| TTP | CD8 | CD80 | FcγRIII-γ |
| TTP | CD8 | CD80 | FcεRIβ |
| TTP | CD8 | CD80 | FcεRIγ |
| TTP | CD8 | CD80 | DAP10 |
| TTP | CD8 | CD80 | DAP12 |
| TTP | CD8 | CD80 | CD32 |
| TTP | CD8 | CD80 | CD79a |
| TTP | CD8 | CD80 | CD79b |
| TTP | CD8 | CD86 | CD8 |
| TTP | CD8 | CD86 | CD3ζ |
| TTP | CD8 | CD86 | CD3δ |
| TTP | CD8 | CD86 | CD3γ |
| TTP | CD8 | CD86 | CD3ε |
| TTP | CD8 | CD86 | FcγRI-γ |
| TTP | CD8 | CD86 | FcγRIII-γ |
| TTP | CD8 | CD86 | FcεRIβ |
| TTP | CD8 | CD86 | FcεRIγ |
| TTP | CD8 | CD86 | DAP10 |
| TTP | CD8 | CD86 | DAP12 |
| TTP | CD8 | CD86 | CD32 |
| TTP | CD8 | CD86 | CD79a |
| TTP | CD8 | CD86 | CD79b |
| TTP | CD8 | OX40 | CD8 |
| TTP | CD8 | OX40 | CD3ζ |
| TTP | CD8 | OX40 | CD3δ |
| TTP | CD8 | OX40 | CD3γ |
| TTP | CD8 | OX40 | CD3ε |
| TTP | CD8 | OX40 | FcγRI-γ |
| TTP | CD8 | OX40 | FcγRIII-γ |
| TTP | CD8 | OX40 | FcεRIβ |
| TTP | CD8 | OX40 | FcεRIγ |
| TTP | CD8 | OX40 | DAP10 |
| TTP | CD8 | OX40 | DAP12 |
| TTP | CD8 | OX40 | CD32 |
| TTP | CD8 | OX40 | CD79a |
| TTP | CD8 | OX40 | CD79b |
| TTP | CD8 | DAP10 | CD8 |
| TTP | CD8 | DAP10 | CD3ζ |
| TTP | CD8 | DAP10 | CD3δ |
| TTP | CD8 | DAP10 | CD3γ |
| TTP | CD8 | DAP10 | CD3ε |
| TTP | CD8 | DAP10 | FcγRI-γ |
| TTP | CD8 | DAP10 | FcγRIII-γ |
| TTP | CD8 | DAP10 | FcεRIβ |
| TTP | CD8 | DAP10 | FcεRIγ |
| TTP | CD8 | DAP10 | DAP10 |
| TTP | CD8 | DAP10 | DAP12 |
| TTP | CD8 | DAP10 | CD32 |
| TTP | CD8 | DAP10 | CD79a |
| TTP | CD8 | DAP10 | CD79b |
| TTP | CD8 | DAP12 | CD8 |
| TTP | CD8 | DAP12 | CD3ζ |
| TTP | CD8 | DAP12 | CD3δ |
| TTP | CD8 | DAP12 | CD3γ |
| TTP | CD8 | DAP12 | CD3ε |
| TTP | CD8 | DAP12 | FcγRI-γ |
| TTP | CD8 | DAP12 | FcγRIII-γ |
| TTP | CD8 | DAP12 | FcεRIβ |
| TTP | CD8 | DAP12 | FcεRIγ |
| TTP | CD8 | DAP12 | DAP10 |
| TTP | CD8 | DAP12 | DAP12 |
| TTP | CD8 | DAP12 | CD32 |
| TTP | CD8 | DAP12 | CD79a |
| TTP | CD8 | DAP12 | CD79b |
| TTP | CD8 | MyD88 | CD8 |
| TTP | CD8 | MyD88 | CD3ζ |
| TTP | CD8 | MyD88 | CD3δ |
| TTP | CD8 | MyD88 | CD3γ |
| TTP | CD8 | MyD88 | CD3ε |
| TTP | CD8 | MyD88 | FcγRI-γ |
| TTP | CD8 | MyD88 | FcγRIII-γ |
| TTP | CD8 | MyD88 | FcεRIβ |
| TTP | CD8 | MyD88 | FcεRIγ |
| TTP | CD8 | MyD88 | DAP10 |
| TTP | CD8 | MyD88 | DAP12 |
| TTP | CD8 | MyD88 | CD32 |
| TTP | CD8 | MyD88 | CD79a |
| TTP | CD8 | MyD88 | CD79b |
| TTP | CD8 | CD7 | CD8 |
| TTP | CD8 | CD7 | CD3ζ |
| TTP | CD8 | CD7 | CD3δ |
| TTP | CD8 | CD7 | CD3γ |
| TTP | CD8 | CD7 | CD3ε |
| TTP | CD8 | CD7 | FcγRI-γ |
| TTP | CD8 | CD7 | FcγRIII-γ |
| TTP | CD8 | CD7 | FcεRIβ |
| TTP | CD8 | CD7 | FcεRIγ |
| TTP | CD8 | CD7 | DAP10 |
| TTP | CD8 | CD7 | DAP12 |
| TTP | CD8 | CD7 | CD32 |
| TTP | CD8 | CD7 | CD79a |
| TTP | CD8 | CD7 | CD79b |
| TTP | CD8 | BTNL3 | CD8 |
| TTP | CD8 | BTNL3 | CD3ζ |
| TTP | CD8 | BTNL3 | CD3δ |
| TTP | CD8 | BTNL3 | CD3γ |
| TTP | CD8 | BTNL3 | CD3ε |
| TTP | CD8 | BTNL3 | FcγRI-γ |
| TTP | CD8 | BTNL3 | FcγRIII-γ |
| TTP | CD8 | BTNL3 | FcεRIβ |
| TTP | CD8 | BTNL3 | FcεRIγ |
| TTP | CD8 | BTNL3 | DAP10 |
| TTP | CD8 | BTNL3 | DAP12 |
| TTP | CD8 | BTNL3 | CD32 |
| TTP | CD8 | BTNL3 | CD79a |
| TTP | CD8 | BTNL3 | CD79b |
| TTP | CD8 | NKG2D | CD8 |
| TTP | CD8 | NKG2D | CD3ζ |
| TTP | CD8 | NKG2D | CD3δ |
| TTP | CD8 | NKG2D | CD3γ |
| TTP | CD8 | NKG2D | CD3ε |
| TTP | CD8 | NKG2D | FcγRI-γ |
| TTP | CD8 | NKG2D | FcγRIII-γ |
| TTP | CD8 | NKG2D | FcεRIβ |
| TTP | CD8 | NKG2D | FcεRIγ |
| TTP | CD8 | NKG2D | DAP10 |
| TTP | CD8 | NKG2D | DAP12 |
| TTP | CD8 | NKG2D | CD32 |
| TTP | CD8 | NKG2D | CD79a |
| TTP | CD8 | NKG2D | CD79b |
| TTP | CD4 | CD28 | CD8 |
| TTP | CD4 | CD28 | CD3ζ |
| TTP | CD4 | CD28 | CD3δ |
| TTP | CD4 | CD28 | CD3γ |
| TTP | CD4 | CD28 | CD3ε |
| TTP | CD4 | CD28 | FcγRI-γ |
| TTP | CD4 | CD28 | FcγRIII-γ |
| TTP | CD4 | CD28 | FcεRIβ |
| TTP | CD4 | CD28 | FcεRIγ |
| TTP | CD4 | CD28 | DAP10 |
| TTP | CD4 | CD28 | DAP12 |
| TTP | CD4 | CD28 | CD32 |
| TTP | CD4 | CD28 | CD79a |
| TTP | CD4 | CD28 | CD79b |
| TTP | CD4 | CD8 | CD8 |
| TTP | CD4 | CD8 | CD3ζ |
| TTP | CD4 | CD8 | CD3δ |
| TTP | CD4 | CD8 | CD3γ |
| TTP | CD4 | CD8 | CD3ε |
| TTP | CD4 | CD8 | FcγRI-γ |
| TTP | CD4 | CD8 | FcγRIII-γ |
| TTP | CD4 | CD8 | FcεRIβ |
| TTP | CD4 | CD8 | FcεRIγ |
| TTP | CD4 | CD8 | DAP10 |
| TTP | CD4 | CD8 | DAP12 |
| TTP | CD4 | CD8 | CD32 |
| TTP | CD4 | CD8 | CD79a |
| TTP | CD4 | CD8 | CD79b |
| TTP | CD4 | CD4 | CD8 |
| TTP | CD4 | CD4 | CD3ζ |
| TTP | CD4 | CD4 | CD3δ |
| TTP | CD4 | CD4 | CD3γ |
| TTP | CD4 | CD4 | CD3ε |
| TTP | CD4 | CD4 | FcγRI-γ |
| TTP | CD4 | CD4 | FcγRIII-γ |
| TTP | CD4 | CD4 | FcεRIβ |
| TTP | CD4 | CD4 | FcεRIγ |
| TTP | CD4 | CD4 | DAP10 |
| TTP | CD4 | CD4 | DAP12 |
| TTP | CD4 | CD4 | CD32 |
| TTP | CD4 | CD4 | CD79a |
| TTP | CD4 | CD4 | CD79b |
| TTP | CD4 | b2c | CD8 |
| TTP | CD4 | b2c | CD3ζ |
| TTP | CD4 | b2c | CD3δ |
| TTP | CD4 | b2c | CD3γ |
| TTP | CD4 | b2c | CD3ε |
| TTP | CD4 | b2c | FcγRI-γ |
| TTP | CD4 | b2c | FcγRIII-γ |
| TTP | CD4 | b2c | FcεRIβ |
| TTP | CD4 | b2c | FcεRIγ |
| TTP | CD4 | b2c | DAP10 |
| TTP | CD4 | b2c | DAP12 |
| TTP | CD4 | b2c | CD32 |
| TTP | CD4 | b2c | CD79a |
| TTP | CD4 | b2c | CD79b |
| TTP | CD4 | CD137/41BB | CD8 |
| TTP | CD4 | CD137/41BB | CD3ζ |
| TTP | CD4 | CD137/41BB | CD3δ |
| TTP | CD4 | CD137/41BB | CD3γ |
| TTP | CD4 | CD137/41BB | CD3ε |
| TTP | CD4 | CD137/41BB | FcγRI-γ |
| TTP | CD4 | CD137/41BB | FcγRIII-γ |
| TTP | CD4 | CD137/41BB | FcεRIβ |
| TTP | CD4 | CD137/41BB | FcεRIγ |
| TTP | CD4 | CD137/41BB | DAP10 |
| TTP | CD4 | CD137/41BB | DAP12 |
| TTP | CD4 | CD137/41BB | CD32 |
| TTP | CD4 | CD137/41BB | CD79a |
| TTP | CD4 | CD137/41BB | CD79b |
| TTP | CD4 | ICOS | CD8 |
| TTP | CD4 | ICOS | CD3ζ |
| TTP | CD4 | ICOS | CD3δ |
| TTP | CD4 | ICOS | CD3γ |
| TTP | CD4 | ICOS | CD3ε |
| TTP | CD4 | ICOS | FcγRI-γ |
| TTP | CD4 | ICOS | FcγRIII-γ |
| TTP | CD4 | ICOS | FcεRIβ |
| TTP | CD4 | ICOS | FcεRIγ |
| TTP | CD4 | ICOS | DAP10 |
| TTP | CD4 | ICOS | DAP12 |
| TTP | CD4 | ICOS | CD32 |
| TTP | CD4 | ICOS | CD79a |
| TTP | CD4 | ICOS | CD79b |
| TTP | CD4 | CD27 | CD8 |
| TTP | CD4 | CD27 | CD3ζ |
| TTP | CD4 | CD27 | CD3δ |
| TTP | CD4 | CD27 | CD3γ |
| TTP | CD4 | CD27 | CD3ε |
| TTP | CD4 | CD27 | FcγRI-γ |
| TTP | CD4 | CD27 | FcγRIII-γ |
| TTP | CD4 | CD27 | FcεRIβ |
| TTP | CD4 | CD27 | FcεRIγ |
| TTP | CD4 | CD27 | DAP10 |
| TTP | CD4 | CD27 | DAP12 |
| TTP | CD4 | CD27 | CD32 |
| TTP | CD4 | CD27 | CD79a |
| TTP | CD4 | CD27 | CD79b |
| TTP | CD4 | CD28δ | CD8 |
| TTP | CD4 | CD28δ | CD3ζ |
| TTP | CD4 | CD28δ | CD3δ |
| TTP | CD4 | CD28δ | CD3γ |
| TTP | CD4 | CD28δ | CD3ε |
| TTP | CD4 | CD28δ | FcγRI-γ |
| TTP | CD4 | CD28δ | FcγRIII-γ |
| TTP | CD4 | CD28δ | FcεRIβ |
| TTP | CD4 | CD28δ | FcεRIγ |
| TTP | CD4 | CD28δ | DAP10 |
| TTP | CD4 | CD28δ | DAP12 |
| TTP | CD4 | CD28δ | CD32 |
| TTP | CD4 | CD28δ | CD79a |
| TTP | CD4 | CD28δ | CD79b |
| TTP | CD4 | CD80 | CD8 |
| TTP | CD4 | CD80 | CD3ζ |
| TTP | CD4 | CD80 | CD3δ |
| TTP | CD4 | CD80 | CD3γ |
| TTP | CD4 | CD80 | CD3ε |
| TTP | CD4 | CD80 | FcγRI-γ |
| TTP | CD4 | CD80 | FcγRIII-γ |
| TTP | CD4 | CD80 | FcεRIβ |
| TTP | CD4 | CD80 | FcεRIγ |
| TTP | CD4 | CD80 | DAP10 |
| TTP | CD4 | CD80 | DAP12 |
| TTP | CD4 | CD80 | CD32 |
| TTP | CD4 | CD80 | CD79a |
| TTP | CD4 | CD80 | CD79b |
| TTP | CD4 | CD86 | CD8 |
| TTP | CD4 | CD86 | CD3ζ |
| TTP | CD4 | CD86 | CD3δ |
| TTP | CD4 | CD86 | CD3γ |
| TTP | CD4 | CD86 | CD3ε |
| TTP | CD4 | CD86 | FcγRI-γ |
| TTP | CD4 | CD86 | FcγRIII-γ |
| TTP | CD4 | CD86 | FcεRIβ |
| TTP | CD4 | CD86 | FcεRIγ |
| TTP | CD4 | CD86 | DAP10 |
| TTP | CD4 | CD86 | DAP12 |
| TTP | CD4 | CD86 | CD32 |
| TTP | CD4 | CD86 | CD79a |
| TTP | CD4 | CD86 | CD79b |
| TTP | CD4 | OX40 | CD8 |
| TTP | CD4 | OX40 | CD3ζ |
| TTP | CD4 | OX40 | CD3δ |
| TTP | CD4 | OX40 | CD3γ |
| TTP | CD4 | OX40 | CD3ε |
| TTP | CD4 | OX40 | FcγRI-γ |
| TTP | CD4 | OX40 | FcγRIII-γ |
| TTP | CD4 | OX40 | FcεRIβ |
| TTP | CD4 | OX40 | FcεRIγ |
| TTP | CD4 | OX40 | DAP10 |
| TTP | CD4 | OX40 | DAP12 |
| TTP | CD4 | OX40 | CD32 |
| TTP | CD4 | OX40 | CD79a |
| TTP | CD4 | OX40 | CD79b |
| TTP | CD4 | DAP10 | CD8 |
| TTP | CD4 | DAP10 | CD3ζ |
| TTP | CD4 | DAP10 | CD3δ |
| TTP | CD4 | DAP10 | CD3γ |
| TTP | CD4 | DAP10 | CD3ε |
| TTP | CD4 | DAP10 | FcγRI-γ |
| TTP | CD4 | DAP10 | FcγRIII-γ |
| TTP | CD4 | DAP10 | FcεRIβ |
| TTP | CD4 | DAP10 | FcεRIγ |
| TTP | CD4 | DAP10 | DAP10 |
| TTP | CD4 | DAP10 | DAP12 |
| TTP | CD4 | DAP10 | CD32 |
| TTP | CD4 | DAP10 | CD79a |
| TTP | CD4 | DAP10 | CD79b |
| TTP | CD4 | DAP12 | CD8 |
| TTP | CD4 | DAP12 | CD3ζ |
| TTP | CD4 | DAP12 | CD3δ |
| TTP | CD4 | DAP12 | CD3γ |
| TTP | CD4 | DAP12 | CD3ε |
| TTP | CD4 | DAP12 | FcγRI-γ |
| TTP | CD4 | DAP12 | FcγRIII-γ |
| TTP | CD4 | DAP12 | FcεRIβ |
| TTP | CD4 | DAP12 | FcεRIγ |
| TTP | CD4 | DAP12 | DAP10 |
| TTP | CD4 | DAP12 | DAP12 |
| TTP | CD4 | DAP12 | CD32 |
| TTP | CD4 | DAP12 | CD79a |
| TTP | CD4 | DAP12 | CD79b |
| TTP | CD4 | MyD88 | CD8 |
| TTP | CD4 | MyD88 | CD3ζ |
| TTP | CD4 | MyD88 | CD3δ |
| TTP | CD4 | MyD88 | CD3γ |
| TTP | CD4 | MyD88 | CD3ε |
| TTP | CD4 | MyD88 | FcγRI-γ |
| TTP | CD4 | MyD88 | FcγRIII-γ |
| TTP | CD4 | MyD88 | FcεRIβ |
| TTP | CD4 | MyD88 | FcεRIγ |
| TTP | CD4 | MyD88 | DAP10 |
| TTP | CD4 | MyD88 | DAP12 |
| TTP | CD4 | MyD88 | CD32 |
| TTP | CD4 | MyD88 | CD79a |
| TTP | CD4 | MyD88 | CD79b |
| TTP | CD4 | CD7 | CD8 |
| TTP | CD4 | CD7 | CD3ζ |
| TTP | CD4 | CD7 | CD3δ |
| TTP | CD4 | CD7 | CD3γ |
| TTP | CD4 | CD7 | CD3ε |
| TTP | CD4 | CD7 | FcγRI-γ |
| TTP | CD4 | CD7 | FcγRIII-γ |
| TTP | CD4 | CD7 | FcεRIβ |
| TTP | CD4 | CD7 | FcεRIγ |
| TTP | CD4 | CD7 | DAP10 |
| TTP | CD4 | CD7 | DAP12 |
| TTP | CD4 | CD7 | CD32 |
| TTP | CD4 | CD7 | CD79a |
| TTP | CD4 | CD7 | CD79b |
| TTP | CD4 | BTNL3 | CD8 |
| TTP | CD4 | BTNL3 | CD3ζ |
| TTP | CD4 | BTNL3 | CD3δ |
| TTP | CD4 | BTNL3 | CD3γ |
| TTP | CD4 | BTNL3 | CD3ε |
| TTP | CD4 | BTNL3 | FcγRI-γ |
| TTP | CD4 | BTNL3 | FcγRIII-γ |
| TTP | CD4 | BTNL3 | FcεRIβ |
| TTP | CD4 | BTNL3 | FcεRIγ |
| TTP | CD4 | BTNL3 | DAP10 |
| TTP | CD4 | BTNL3 | DAP12 |
| TTP | CD4 | BTNL3 | CD32 |
| TTP | CD4 | BTNL3 | CD79a |
| TTP | CD4 | BTNL3 | CD79b |
| TTP | CD4 | NKG2D | CD8 |
| TTP | CD4 | NKG2D | CD3ζ |
| TTP | CD4 | NKG2D | CD3δ |
| TTP | CD4 | NKG2D | CD3γ |
| TTP | CD4 | NKG2D | CD3ε |
| TTP | CD4 | NKG2D | FcγRI-γ |
| TTP | CD4 | NKG2D | FcγRIII-γ |
| TTP | CD4 | NKG2D | FcεRIβ |
| TTP | CD4 | NKG2D | FcεRIγ |
| TTP | CD4 | NKG2D | DAP10 |
| TTP | CD4 | NKG2D | DAP12 |
| TTP | CD4 | NKG2D | CD32 |
| TTP | CD4 | NKG2D | CD79a |
| TTP | CD4 | NKG2D | CD79b |
| TTP | b2c | CD28 | CD8 |
| TTP | b2c | CD28 | CD3ζ |
| TTP | b2c | CD28 | CD3δ |
| TTP | b2c | CD28 | CD3γ |
| TTP | b2c | CD28 | CD3ε |
| TTP | b2c | CD28 | FcγRI-γ |
| TTP | b2c | CD28 | FcγRIII-γ |
| TTP | b2c | CD28 | FcεRIβ |
| TTP | b2c | CD28 | FcεRIγ |
| TTP | b2c | CD28 | DAP10 |
| TTP | b2c | CD28 | DAP12 |
| TTP | b2c | CD28 | CD32 |
| TTP | b2c | CD28 | CD79a |
| TTP | b2c | CD28 | CD79b |
| TTP | b2c | CD8 | CD8 |
| TTP | b2c | CD8 | CD3ζ |
| TTP | b2c | CD8 | CD3δ |
| TTP | b2c | CD8 | CD3γ |
| TTP | b2c | CD8 | CD3ε |
| TTP | b2c | CD8 | FcγRI-γ |
| TTP | b2c | CD8 | FcγRIII-γ |
| TTP | b2c | CD8 | FcεRIβ |
| TTP | b2c | CD8 | FcεRIγ |
| TTP | b2c | CD8 | DAP10 |
| TTP | b2c | CD8 | DAP12 |
| TTP | b2c | CD8 | CD32 |
| TTP | b2c | CD8 | CD79a |
| TTP | b2c | CD8 | CD79b |
| TTP | b2c | CD4 | CD8 |
| TTP | b2c | CD4 | CD3ζ |
| TTP | b2c | CD4 | CD3δ |
| TTP | b2c | CD4 | CD3γ |
| TTP | b2c | CD4 | CD3ε |
| TTP | b2c | CD4 | FcγRI-γ |
| TTP | b2c | CD4 | FcγRIII-γ |
| TTP | b2c | CD4 | FcεRIβ |
| TTP | b2c | CD4 | FcεRIγ |
| TTP | b2c | CD4 | DAP10 |
| TTP | b2c | CD4 | DAP12 |
| TTP | b2c | CD4 | CD32 |
| TTP | b2c | CD4 | CD79a |
| TTP | b2c | CD4 | CD79b |
| TTP | b2c | b2c | CD8 |
| TTP | b2c | b2c | CD3ζ |
| TTP | b2c | b2c | CD3δ |
| TTP | b2c | b2c | CD3γ |
| TTP | b2c | b2c | CD3ε |
| TTP | b2c | b2c | FcγRI-γ |
| TTP | b2c | b2c | FcγRIII-γ |
| TTP | b2c | b2c | FcεRIβ |
| TTP | b2c | b2c | FcεRIγ |
| TTP | b2c | b2c | DAP10 |
| TTP | b2c | b2c | DAP12 |
| TTP | b2c | b2c | CD32 |
| TTP | b2c | b2c | CD79a |
| TTP | b2c | b2c | CD79b |
| TTP | b2c | CD137/41BB | CD8 |
| TTP | b2c | CD137/41BB | CD3ζ |
| TTP | b2c | CD137/41BB | CD3δ |
| TTP | b2c | CD137/41BB | CD3γ |
| TTP | b2c | CD137/41BB | CD3ε |
| TTP | b2c | CD137/41BB | FcγRI-γ |
| TTP | b2c | CD137/41BB | FcγRIII-γ |
| TTP | b2c | CD137/41BB | FcεRIβ |
| TTP | b2c | CD137/41BB | FcεRIγ |
| TTP | b2c | CD137/41BB | DAP10 |
| TTP | b2c | CD137/41BB | DAP12 |
| TTP | b2c | CD137/41BB | CD32 |
| TTP | b2c | CD137/41BB | CD79a |
| TTP | b2c | CD137/41BB | CD79b |
| TTP | b2c | ICOS | CD8 |
| TTP | b2c | ICOS | CD3ζ |
| TTP | b2c | ICOS | CD3δ |
| TTP | b2c | ICOS | CD3γ |
| TTP | b2c | ICOS | CD3ε |
| TTP | b2c | ICOS | FcγRI-γ |
| TTP | b2c | ICOS | FcγRIII-γ |
| TTP | b2c | ICOS | FcεRIβ |
| TTP | b2c | ICOS | FcεRIγ |
| TTP | b2c | ICOS | DAP10 |
| TTP | b2c | ICOS | DAP12 |
| TTP | b2c | ICOS | CD32 |
| TTP | b2c | ICOS | CD79a |
| TTP | b2c | ICOS | CD79b |
| TTP | b2c | CD27 | CD8 |
| TTP | b2c | CD27 | CD3ζ |
| TTP | b2c | CD27 | CD3δ |
| TTP | b2c | CD27 | CD3γ |
| TTP | b2c | CD27 | CD3ε |
| TTP | b2c | CD27 | FcγRI-γ |
| TTP | b2c | CD27 | FcγRIII-γ |
| TTP | b2c | CD27 | FcεRIβ |
| TTP | b2c | CD27 | FcεRIγ |
| TTP | b2c | CD27 | DAP10 |
| TTP | b2c | CD27 | DAP12 |
| TTP | b2c | CD27 | CD32 |
| TTP | b2c | CD27 | CD79a |
| TTP | b2c | CD27 | CD79b |
| TTP | b2c | CD28δ | CD8 |
| TTP | b2c | CD28δ | CD3ζ |
| TTP | b2c | CD28δ | CD3δ |
| TTP | b2c | CD28δ | CD3γ |
| TTP | b2c | CD28δ | CD3ε |
| TTP | b2c | CD28δ | FcγRI-γ |
| TTP | b2c | CD28δ | FcγRIII-γ |
| TTP | b2c | CD28δ | FcεRIβ |
| TTP | b2c | CD28δ | FcεRIγ |
| TTP | b2c | CD28δ | DAP10 |
| TTP | b2c | CD28δ | DAP12 |
| TTP | b2c | CD28δ | CD32 |
| TTP | b2c | CD28δ | CD79a |
| TTP | b2c | CD28δ | CD79b |
| TTP | b2c | CD80 | CD8 |
| TTP | b2c | CD80 | CD3ζ |
| TTP | b2c | CD80 | CD3δ |
| TTP | b2c | CD80 | CD3γ |
| TTP | b2c | CD80 | CD3ε |
| TTP | b2c | CD80 | FcγRI-γ |
| TTP | b2c | CD80 | FcγRIII-γ |
| TTP | b2c | CD80 | FcεRIβ |
| TTP | b2c | CD80 | FcεRIγ |
| TTP | b2c | CD80 | DAP10 |
| TTP | b2c | CD80 | DAP12 |
| TTP | b2c | CD80 | CD32 |
| TTP | b2c | CD80 | CD79a |
| TTP | b2c | CD80 | CD79b |
| TTP | b2c | CD86 | CD8 |
| TTP | b2c | CD86 | CD3ζ |
| TTP | b2c | CD86 | CD3δ |
| TTP | b2c | CD86 | CD3γ |
| TTP | b2c | CD86 | CD3ε |
| TTP | b2c | CD86 | FcγRI-γ |
| TTP | b2c | CD86 | FcγRIII-γ |
| TTP | b2c | CD86 | FcεRIβ |
| TTP | b2c | CD86 | FcεRIγ |
| TTP | b2c | CD86 | DAP10 |
| TTP | b2c | CD86 | DAP12 |
| TTP | b2c | CD86 | CD32 |
| TTP | b2c | CD86 | CD79a |
| TTP | b2c | CD86 | CD79b |
| TTP | b2c | OX40 | CD8 |
| TTP | b2c | OX40 | CD3ζ |
| TTP | b2c | OX40 | CD3δ |
| TTP | b2c | OX40 | CD3γ |
| TTP | b2c | OX40 | CD3ε |
| TTP | b2c | OX40 | FcγRI-γ |
| TTP | b2c | OX40 | FcγRIII-γ |
| TTP | b2c | OX40 | FcεRIβ |
| TTP | b2c | OX40 | FcεRIγ |
| TTP | b2c | OX40 | DAP10 |
| TTP | b2c | OX40 | DAP12 |
| TTP | b2c | OX40 | CD32 |
| TTP | b2c | OX40 | CD79a |
| TTP | b2c | OX40 | CD79b |
| TTP | b2c | DAP10 | CD8 |
| TTP | b2c | DAP10 | CD3ζ |
| TTP | b2c | DAP10 | CD3δ |
| TTP | b2c | DAP10 | CD3γ |
| TTP | b2c | DAP10 | CD3ε |
| TTP | b2c | DAP10 | FcγRI-γ |
| TTP | b2c | DAP10 | FcγRIII-γ |
| TTP | b2c | DAP10 | FcεRIβ |
| TTP | b2c | DAP10 | FcεRIγ |
| TTP | b2c | DAP10 | DAP10 |
| TTP | b2c | DAP10 | DAP12 |
| TTP | b2c | DAP10 | CD32 |
| TTP | b2c | DAP10 | CD79a |
| TTP | b2c | DAP10 | CD79b |
| TTP | b2c | DAP12 | CD8 |
| TTP | b2c | DAP12 | CD3ζ |
| TTP | b2c | DAP12 | CD3δ |
| TTP | b2c | DAP12 | CD3γ |
| TTP | b2c | DAP12 | CD3ε |
| TTP | b2c | DAP12 | FcγRI-γ |
| TTP | b2c | DAP12 | FcγRIII-γ |
| TTP | b2c | DAP12 | FcεRIβ |
| TTP | b2c | DAP12 | FcεRIγ |
| TTP | b2c | DAP12 | DAP10 |
| TTP | b2c | DAP12 | DAP12 |
| TTP | b2c | DAP12 | CD32 |
| TTP | b2c | DAP12 | CD79a |
| TTP | b2c | DAP12 | CD79b |
| TTP | b2c | MyD88 | CD8 |
| TTP | b2c | MyD88 | CD3ζ |
| TTP | b2c | MyD88 | CD3δ |
| TTP | b2c | MyD88 | CD3γ |
| TTP | b2c | MyD88 | CD3ε |
| TTP | b2c | MyD88 | FcγRI-γ |
| TTP | b2c | MyD88 | FcγRIII-γ |
| TTP | b2c | MyD88 | FcεRIβ |
| TTP | b2c | MyD88 | FcεRIγ |
| TTP | b2c | MyD88 | DAP10 |
| TTP | b2c | MyD88 | DAP12 |
| TTP | b2c | MyD88 | CD32 |
| TTP | b2c | MyD88 | CD79a |
| TTP | b2c | MyD88 | CD79b |
| TTP | b2c | CD7 | CD8 |
| TTP | b2c | CD7 | CD3ζ |
| TTP | b2c | CD7 | CD3δ |
| TTP | b2c | CD7 | CD3γ |
| TTP | b2c | CD7 | CD3ε |
| TTP | b2c | CD7 | FcγRI-γ |
| TTP | b2c | CD7 | FcγRIII-γ |
| TTP | b2c | CD7 | FcεRIβ |
| TTP | b2c | CD7 | FcεRIγ |
| TTP | b2c | CD7 | DAP10 |
| TTP | b2c | CD7 | DAP12 |
| TTP | b2c | CD7 | CD32 |
| TTP | b2c | CD7 | CD79a |
| TTP | b2c | CD7 | CD79b |
| TTP | b2c | BTNL3 | CD8 |
| TTP | b2c | BTNL3 | CD3ζ |
| TTP | b2c | BTNL3 | CD3δ |
| TTP | b2c | BTNL3 | CD3γ |
| TTP | b2c | BTNL3 | CD3ε |
| TTP | b2c | BTNL3 | FcγRI-γ |
| TTP | b2c | BTNL3 | FcγRIII-γ |
| TTP | b2c | BTNL3 | FcεRIβ |
| TTP | b2c | BTNL3 | FcεRIγ |
| TTP | b2c | BTNL3 | DAP10 |
| TTP | b2c | BTNL3 | DAP12 |
| TTP | b2c | BTNL3 | CD32 |
| TTP | b2c | BTNL3 | CD79a |
| TTP | b2c | BTNL3 | CD79b |
| TTP | b2c | NKG2D | CD8 |
| TTP | b2c | NKG2D | CD3ζ |
| TTP | b2c | NKG2D | CD3δ |
| TTP | b2c | NKG2D | CD3γ |
| TTP | b2c | NKG2D | CD3ε |
| TTP | b2c | NKG2D | FcγRI-γ |
| TTP | b2c | NKG2D | FcγRIII-γ |
| TTP | b2c | NKG2D | FcεRIβ |
| TTP | b2c | NKG2D | FcεRIγ |
| TTP | b2c | NKG2D | DAP10 |
| TTP | b2c | NKG2D | DAP12 |
| TTP | b2c | NKG2D | CD32 |
| TTP | b2c | NKG2D | CD79a |
| TTP | b2c | NKG2D | CD79b |
| TTP | CD137/41BB | CD28 | CD8 |
| TTP | CD137/41BB | CD28 | CD3ζ |
| TTP | CD137/41BB | CD28 | CD3δ |
| TTP | CD137/41BB | CD28 | CD3γ |
| TTP | CD137/41BB | CD28 | CD3ε |
| TTP | CD137/41BB | CD28 | FcγRI-γ |
| TTP | CD137/41BB | CD28 | FcγRIII-γ |
| TTP | CD137/41BB | CD28 | FcεRIβ |
| TTP | CD137/41BB | CD28 | FcεRIγ |
| TTP | CD137/41BB | CD28 | DAP10 |
| TTP | CD137/41BB | CD28 | DAP12 |
| TTP | CD137/41BB | CD28 | CD32 |
| TTP | CD137/41BB | CD28 | CD79a |
| TTP | CD137/41BB | CD28 | CD79b |
| TTP | CD137/41BB | CD8 | CD8 |
| TTP | CD137/41BB | CD8 | CD3ζ |
| TTP | CD137/41BB | CD8 | CD3δ |
| TTP | CD137/41BB | CD8 | CD3γ |
| TTP | CD137/41BB | CD8 | CD3ε |
| TTP | CD137/41BB | CD8 | FcγRI-γ |
| TTP | CD137/41BB | CD8 | FcγRIII-γ |
| TTP | CD137/41BB | CD8 | FcεRIβ |
| TTP | CD137/41BB | CD8 | FcεRIγ |
| TTP | CD137/41BB | CD8 | DAP10 |
| TTP | CD137/41BB | CD8 | DAP12 |
| TTP | CD137/41BB | CD8 | CD32 |
| TTP | CD137/41BB | CD8 | CD79a |
| TTP | CD137/41BB | CD8 | CD79b |
| TTP | CD137/41BB | CD4 | CD8 |
| TTP | CD137/41BB | CD4 | CD3ζ |
| TTP | CD137/41BB | CD4 | CD3δ |
| TTP | CD137/41BB | CD4 | CD3γ |
| TTP | CD137/41BB | CD4 | CD3ε |
| TTP | CD137/41BB | CD4 | FcγRI-γ |
| TTP | CD137/41BB | CD4 | FcγRIII-γ |
| TTP | CD137/41BB | CD4 | FcεRIβ |
| TTP | CD137/41BB | CD4 | FcεRIγ |
| TTP | CD137/41BB | CD4 | DAP10 |
| TTP | CD137/41BB | CD4 | DAP12 |
| TTP | CD137/41BB | CD4 | CD32 |
| TTP | CD137/41BB | CD4 | CD79a |
| TTP | CD137/41BB | CD4 | CD79b |
| TTP | CD137/41BB | b2c | CD8 |
| TTP | CD137/41BB | b2c | CD3ζ |
| TTP | CD137/41BB | b2c | CD3δ |
| TTP | CD137/41BB | b2c | CD3γ |
| TTP | CD137/41BB | b2c | CD3ε |
| TTP | CD137/41BB | b2c | FcγRI-γ |
| TTP | CD137/41BB | b2c | FcγRIII-γ |
| TTP | CD137/41BB | b2c | FcεRIβ |
| TTP | CD137/41BB | b2c | FcεRIγ |
| TTP | CD137/41BB | b2c | DAP10 |
| TTP | CD137/41BB | b2c | DAP12 |
| TTP | CD137/41BB | b2c | CD32 |
| TTP | CD137/41BB | b2c | CD79a |
| TTP | CD137/41BB | b2c | CD79b |
| TTP | CD137/41BB | CD137/41BB | CD8 |
| TTP | CD137/41BB | CD137/41BB | CD3ζ |
| TTP | CD137/41BB | CD137/41BB | CD3δ |
| TTP | CD137/41BB | CD137/41BB | CD3γ |
| TTP | CD137/41BB | CD137/41BB | CD3ε |
| TTP | CD137/41BB | CD137/41BB | FcγRI-γ |
| TTP | CD137/41BB | CD137/41BB | FcγRIII-γ |
| TTP | CD137/41BB | CD137/41BB | FcεRIβ |
| TTP | CD137/41BB | CD137/41BB | FcεRIγ |
| TTP | CD137/41BB | CD137/41BB | DAP10 |
| TTP | CD137/41BB | CD137/41BB | DAP12 |
| TTP | CD137/41BB | CD137/41BB | CD32 |
| TTP | CD137/41BB | CD137/41BB | CD79a |
| TTP | CD137/41BB | CD137/41BB | CD79b |
| TTP | CD137/41BB | ICOS | CD8 |
| TTP | CD137/41BB | ICOS | CD3ζ |
| TTP | CD137/41BB | ICOS | CD3δ |
| TTP | CD137/41BB | ICOS | CD3γ |
| TTP | CD137/41BB | ICOS | CD3ε |
| TTP | CD137/41BB | ICOS | FcγRI-γ |
| TTP | CD137/41BB | ICOS | FcγRIII-γ |
| TTP | CD137/41BB | ICOS | FcεRIβ |
| TTP | CD137/41BB | ICOS | FcεRIγ |
| TTP | CD137/41BB | ICOS | DAP10 |
| TTP | CD137/41BB | ICOS | DAP12 |
| TTP | CD137/41BB | ICOS | CD32 |
| TTP | CD137/41BB | ICOS | CD79a |
| TTP | CD137/41BB | ICOS | CD79b |
| TTP | CD137/41BB | CD27 | CD8 |
| TTP | CD137/41BB | CD27 | CD3ζ |
| TTP | CD137/41BB | CD27 | CD3δ |
| TTP | CD137/41BB | CD27 | CD3γ |
| TTP | CD137/41BB | CD27 | CD3ε |
| TTP | CD137/41BB | CD27 | FcγRI-γ |
| TTP | CD137/41BB | CD27 | FcγRIII-γ |
| TTP | CD137/41BB | CD27 | FcεRIβ |
| TTP | CD137/41BB | CD27 | FcεRIγ |
| TTP | CD137/41BB | CD27 | DAP10 |
| TTP | CD137/41BB | CD27 | DAP12 |
| TTP | CD137/41BB | CD27 | CD32 |
| TTP | CD137/41BB | CD27 | CD79a |
| TTP | CD137/41BB | CD27 | CD79b |
| TTP | CD137/41BB | CD28δ | CD8 |
| TTP | CD137/41BB | CD28δ | CD3ζ |
| TTP | CD137/41BB | CD28δ | CD3δ |
| TTP | CD137/41BB | CD28δ | CD3γ |
| TTP | CD137/41BB | CD28δ | CD3ε |
| TTP | CD137/41BB | CD28δ | FcγRI-γ |
| TTP | CD137/41BB | CD28δ | FcγRIII-γ |
| TTP | CD137/41BB | CD28δ | FcεRIβ |
| TTP | CD137/41BB | CD28δ | FcεRIγ |
| TTP | CD137/41BB | CD28δ | DAP10 |
| TTP | CD137/41BB | CD28δ | DAP12 |
| TTP | CD137/41BB | CD28δ | CD32 |
| TTP | CD137/41BB | CD28δ | CD79a |
| TTP | CD137/41BB | CD28δ | CD79b |
| TTP | CD137/41BB | CD80 | CD8 |
| TTP | CD137/41BB | CD80 | CD3ζ |
| TTP | CD137/41BB | CD80 | CD3δ |
| TTP | CD137/41BB | CD80 | CD3γ |
| TTP | CD137/41BB | CD80 | CD3ε |
| TTP | CD137/41BB | CD80 | FcγRI-γ |
| TTP | CD137/41BB | CD80 | FcγRIII-γ |
| TTP | CD137/41BB | CD80 | FcεRIβ |
| TTP | CD137/41BB | CD80 | FcεRIγ |
| TTP | CD137/41BB | CD80 | DAP10 |
| TTP | CD137/41BB | CD80 | DAP12 |
| TTP | CD137/41BB | CD80 | CD32 |
| TTP | CD137/41BB | CD80 | CD79a |
| TTP | CD137/41BB | CD80 | CD79b |
| TTP | CD137/41BB | CD86 | CD8 |
| TTP | CD137/41BB | CD86 | CD3ζ |
| TTP | CD137/41BB | CD86 | CD3δ |
| TTP | CD137/41BB | CD86 | CD3γ |
| TTP | CD137/41BB | CD86 | CD3ε |
| TTP | CD137/41BB | CD86 | FcγRI-γ |
| TTP | CD137/41BB | CD86 | FcγRIII-γ |
| TTP | CD137/41BB | CD86 | FcεRIβ |
| TTP | CD137/41BB | CD86 | FcεRIγ |
| TTP | CD137/41BB | CD86 | DAP10 |
| TTP | CD137/41BB | CD86 | DAP12 |
| TTP | CD137/41BB | CD86 | CD32 |
| TTP | CD137/41BB | CD86 | CD79a |
| TTP | CD137/41BB | CD86 | CD79b |
| TTP | CD137/41BB | OX40 | CD8 |
| TTP | CD137/41BB | OX40 | CD3ζ |
| TTP | CD137/41BB | OX40 | CD3δ |
| TTP | CD137/41BB | OX40 | CD3γ |
| TTP | CD137/41BB | OX40 | CD3ε |
| TTP | CD137/41BB | OX40 | FcγRI-γ |
| TTP | CD137/41BB | OX40 | FcγRIII-γ |
| TTP | CD137/41BB | OX40 | FcεRIβ |
| TTP | CD137/41BB | OX40 | FcεRIγ |
| TTP | CD137/41BB | OX40 | DAP10 |
| TTP | CD137/41BB | OX40 | DAP12 |
| TTP | CD137/41BB | OX40 | CD32 |
| TTP | CD137/41BB | OX40 | CD79a |
| TTP | CD137/41BB | OX40 | CD79b |
| TTP | CD137/41BB | DAP10 | CD8 |
| TTP | CD137/41BB | DAP10 | CD3ζ |
| TTP | CD137/41BB | DAP10 | CD3δ |
| TTP | CD137/41BB | DAP10 | CD3γ |
| TTP | CD137/41BB | DAP10 | CD3ε |
| TTP | CD137/41BB | DAP10 | FcγRI-γ |
| TTP | CD137/41BB | DAP10 | FcγRIII-γ |
| TTP | CD137/41BB | DAP10 | FcεRIβ |
| TTP | CD137/41BB | DAP10 | FcεRIγ |
| TTP | CD137/41BB | DAP10 | DAP10 |
| TTP | CD137/41BB | DAP10 | DAP12 |
| TTP | CD137/41BB | DAP10 | CD32 |
| TTP | CD137/41BB | DAP10 | CD79a |
| TTP | CD137/41BB | DAP10 | CD79b |
| TTP | CD137/41BB | DAP12 | CD8 |
| TTP | CD137/41BB | DAP12 | CD3ζ |
| TTP | CD137/41BB | DAP12 | CD3δ |
| TTP | CD137/41BB | DAP12 | CD3γ |
| TTP | CD137/41BB | DAP12 | CD3ε |
| TTP | CD137/41BB | DAP12 | FcγRI-γ |
| TTP | CD137/41BB | DAP12 | FcγRIII-γ |
| TTP | CD137/41BB | DAP12 | FcεRIβ |
| TTP | CD137/41BB | DAP12 | FcεRIγ |
| TTP | CD137/41BB | DAP12 | DAP10 |
| TTP | CD137/41BB | DAP12 | DAP12 |
| TTP | CD137/41BB | DAP12 | CD32 |
| TTP | CD137/41BB | DAP12 | CD79a |
| TTP | CD137/41BB | DAP12 | CD79b |
| TTP | CD137/41BB | MyD88 | CD8 |
| TTP | CD137/41BB | MyD88 | CD3ζ |
| TTP | CD137/41BB | MyD88 | CD3δ |
| TTP | CD137/41BB | MyD88 | CD3γ |
| TTP | CD137/41BB | MyD88 | CD3ε |
| TTP | CD137/41BB | MyD88 | FcγRI-γ |
| TTP | CD137/41BB | MyD88 | FcγRIII-γ |
| TTP | CD137/41BB | MyD88 | FcεRIβ |
| TTP | CD137/41BB | MyD88 | FcεRIγ |
| TTP | CD137/41BB | MyD88 | DAP10 |
| TTP | CD137/41BB | MyD88 | DAP12 |
| TTP | CD137/41BB | MyD88 | CD32 |
| TTP | CD137/41BB | MyD88 | CD79a |
| TTP | CD137/41BB | MyD88 | CD79b |
| TTP | CD137/41BB | CD7 | CD8 |
| TTP | CD137/41BB | CD7 | CD3ζ |
| TTP | CD137/41BB | CD7 | CD3δ |
| TTP | CD137/41BB | CD7 | CD3γ |
| TTP | CD137/41BB | CD7 | CD3ε |
| TTP | CD137/41BB | CD7 | FcγRI-γ |
| TTP | CD137/41BB | CD7 | FcγRIII-γ |
| TTP | CD137/41BB | CD7 | FcεRIβ |
| TTP | CD137/41BB | CD7 | FcεRIγ |
| TTP | CD137/41BB | CD7 | DAP10 |
| TTP | CD137/41BB | CD7 | DAP12 |
| TTP | CD137/41BB | CD7 | CD32 |
| TTP | CD137/41BB | CD7 | CD79a |
| TTP | CD137/41BB | CD7 | CD79b |
| TTP | CD137/41BB | BTNL3 | CD8 |
| TTP | CD137/41BB | BTNL3 | CD3ζ |
| TTP | CD137/41BB | BTNL3 | CD3δ |
| TTP | CD137/41BB | BTNL3 | CD3γ |
| TTP | CD137/41BB | BTNL3 | CD3ε |
| TTP | CD137/41BB | BTNL3 | FcγRI-γ |
| TTP | CD137/41BB | BTNL3 | FcγRIII-γ |
| TTP | CD137/41BB | BTNL3 | FcεRIβ |
| TTP | CD137/41BB | BTNL3 | FcεRIγ |
| TTP | CD137/41BB | BTNL3 | DAP10 |
| TTP | CD137/41BB | BTNL3 | DAP12 |
| TTP | CD137/41BB | BTNL3 | CD32 |
| TTP | CD137/41BB | BTNL3 | CD79a |
| TTP | CD137/41BB | BTNL3 | CD79b |
| TTP | CD137/41BB | NKG2D | CD8 |
| TTP | CD137/41BB | NKG2D | CD3ζ |
| TTP | CD137/41BB | NKG2D | CD3δ |
| TTP | CD137/41BB | NKG2D | CD3γ |
| TTP | CD137/41BB | NKG2D | CD3ε |
| TTP | CD137/41BB | NKG2D | FcγRI-γ |
| TTP | CD137/41BB | NKG2D | FcγRIII-γ |
| TTP | CD137/41BB | NKG2D | FcεRIβ |
| TTP | CD137/41BB | NKG2D | FcεRIγ |
| TTP | CD137/41BB | NKG2D | DAP10 |
| TTP | CD137/41BB | NKG2D | DAP12 |
| TTP | CD137/41BB | NKG2D | CD32 |
| TTP | CD137/41BB | NKG2D | CD79a |
| TTP | CD137/41BB | NKG2D | CD79b |
| TTP | ICOS | CD28 | CD8 |
| TTP | ICOS | CD28 | CD3ζ |
| TTP | ICOS | CD28 | CD3δ |
| TTP | ICOS | CD28 | CD3γ |
| TTP | ICOS | CD28 | CD3ε |
| TTP | ICOS | CD28 | FcγRI-γ |
| TTP | ICOS | CD28 | FcγRIII-γ |
| TTP | ICOS | CD28 | FcεRIβ |
| TTP | ICOS | CD28 | FcεRIγ |
| TTP | ICOS | CD28 | DAP10 |
| TTP | ICOS | CD28 | DAP12 |
| TTP | ICOS | CD28 | CD32 |
| TTP | ICOS | CD28 | CD79a |
| TTP | ICOS | CD28 | CD79b |
| TTP | ICOS | CD8 | CD8 |
| TTP | ICOS | CD8 | CD3ζ |
| TTP | ICOS | CD8 | CD3δ |
| TTP | ICOS | CD8 | CD3γ |
| TTP | ICOS | CD8 | CD3ε |
| TTP | ICOS | CD8 | FcγRI-γ |
| TTP | ICOS | CD8 | FcγRIII-γ |
| TTP | ICOS | CD8 | FcεRIβ |
| TTP | ICOS | CD8 | FcεRIγ |
| TTP | ICOS | CD8 | DAP10 |
| TTP | ICOS | CD8 | DAP12 |
| TTP | ICOS | CD8 | CD32 |
| TTP | ICOS | CD8 | CD79a |
| TTP | ICOS | CD8 | CD79b |
| TTP | ICOS | CD4 | CD8 |
| TTP | ICOS | CD4 | CD3ζ |
| TTP | ICOS | CD4 | CD3δ |
| TTP | ICOS | CD4 | CD3γ |
| TTP | ICOS | CD4 | CD3ε |
| TTP | ICOS | CD4 | FcγRI-γ |
| TTP | ICOS | CD4 | FcγRIII-γ |
| TTP | ICOS | CD4 | FcεRIβ |
| TTP | ICOS | CD4 | FcεRIγ |
| TTP | ICOS | CD4 | DAP10 |
| TTP | ICOS | CD4 | DAP12 |
| TTP | ICOS | CD4 | CD32 |
| TTP | ICOS | CD4 | CD79a |
| TTP | ICOS | CD4 | CD79b |
| TTP | ICOS | b2c | CD8 |
| TTP | ICOS | b2c | CD3ζ |
| TTP | ICOS | b2c | CD3δ |
| TTP | ICOS | b2c | CD3γ |
| TTP | ICOS | b2c | CD3ε |
| TTP | ICOS | b2c | FcγRI-γ |
| TTP | ICOS | b2c | FcγRIII-γ |
| TTP | ICOS | b2c | FcεRIβ |
| TTP | ICOS | b2c | FcεRIγ |
| TTP | ICOS | b2c | DAP10 |
| TTP | ICOS | b2c | DAP12 |
| TTP | ICOS | b2c | CD32 |
| TTP | ICOS | b2c | CD79a |
| TTP | ICOS | b2c | CD79b |
| TTP | ICOS | CD137/41BB | CD8 |
| TTP | ICOS | CD137/41BB | CD3ζ |
| TTP | ICOS | CD137/41BB | CD3δ |
| TTP | ICOS | CD137/41BB | CD3γ |
| TTP | ICOS | CD137/41BB | CD3ε |
| TTP | ICOS | CD137/41BB | FcγRI-γ |
| TTP | ICOS | CD137/41BB | FcγRIII-γ |
| TTP | ICOS | CD137/41BB | FcεRIβ |
| TTP | ICOS | CD137/41BB | FcεRIγ |
| TTP | ICOS | CD137/41BB | DAP10 |
| TTP | ICOS | CD137/41BB | DAP12 |
| TTP | ICOS | CD137/41BB | CD32 |
| TTP | ICOS | CD137/41BB | CD79a |
| TTP | ICOS | CD137/41BB | CD79b |
| TTP | ICOS | ICOS | CD8 |
| TTP | ICOS | ICOS | CD3ζ |
| TTP | ICOS | ICOS | CD3δ |
| TTP | ICOS | ICOS | CD3γ |
| TTP | ICOS | ICOS | CD3ε |
| TTP | ICOS | ICOS | FcγRI-γ |
| TTP | ICOS | ICOS | FcγRIII-γ |
| TTP | ICOS | ICOS | FcεRIβ |
| TTP | ICOS | ICOS | FcεRIγ |
| TTP | ICOS | ICOS | DAP10 |
| TTP | ICOS | ICOS | DAP12 |
| TTP | ICOS | ICOS | CD32 |
| TTP | ICOS | ICOS | CD79a |
| TTP | ICOS | ICOS | CD79b |
| TTP | ICOS | CD27 | CD8 |
| TTP | ICOS | CD27 | CD3ζ |
| TTP | ICOS | CD27 | CD3δ |
| TTP | ICOS | CD27 | CD3γ |
| TTP | ICOS | CD27 | CD3ε |
| TTP | ICOS | CD27 | FcγRI-γ |
| TTP | ICOS | CD27 | FcγRIII-γ |
| TTP | ICOS | CD27 | FcεRIβ |
| TTP | ICOS | CD27 | FcεRIγ |
| TTP | ICOS | CD27 | DAP10 |
| TTP | ICOS | CD27 | DAP12 |
| TTP | ICOS | CD27 | CD32 |
| TTP | ICOS | CD27 | CD79a |
| TTP | ICOS | CD27 | CD79b |
| TTP | ICOS | CD28δ | CD8 |
| TTP | ICOS | CD28δ | CD3ζ |
| TTP | ICOS | CD28δ | CD3δ |
| TTP | ICOS | CD28δ | CD3γ |
| TTP | ICOS | CD28δ | CD3ε |
| TTP | ICOS | CD28δ | FcγRI-γ |
| TTP | ICOS | CD28δ | FcγRIII-γ |
| TTP | ICOS | CD28δ | FcεRIβ |
| TTP | ICOS | CD28δ | FcεRIγ |
| TTP | ICOS | CD28δ | DAP10 |
| TTP | ICOS | CD28δ | DAP12 |
| TTP | ICOS | CD28δ | CD32 |
| TTP | ICOS | CD28δ | CD79a |
| TTP | ICOS | CD28δ | CD79b |
| TTP | ICOS | CD80 | CD8 |
| TTP | ICOS | CD80 | CD3ζ |
| TTP | ICOS | CD80 | CD3δ |
| TTP | ICOS | CD80 | CD3γ |
| TTP | ICOS | CD80 | CD3ε |
| TTP | ICOS | CD80 | FcγRI-γ |
| TTP | ICOS | CD80 | FcγRIII-γ |
| TTP | ICOS | CD80 | FcεRIβ |
| TTP | ICOS | CD80 | FcεRIγ |
| TTP | ICOS | CD80 | DAP10 |
| TTP | ICOS | CD80 | DAP12 |
| TTP | ICOS | CD80 | CD32 |
| TTP | ICOS | CD80 | CD79a |
| TTP | ICOS | CD80 | CD79b |
| TTP | ICOS | CD86 | CD8 |
| TTP | ICOS | CD86 | CD3ζ |
| TTP | ICOS | CD86 | CD3δ |
| TTP | ICOS | CD86 | CD3γ |
| TTP | ICOS | CD86 | CD3ε |
| TTP | ICOS | CD86 | FcγRI-γ |
| TTP | ICOS | CD86 | FcγRIII-γ |
| TTP | ICOS | CD86 | FcεRIβ |
| TTP | ICOS | CD86 | FcεRIγ |
| TTP | ICOS | CD86 | DAP10 |
| TTP | ICOS | CD86 | DAP12 |
| TTP | ICOS | CD86 | CD32 |
| TTP | ICOS | CD86 | CD79a |
| TTP | ICOS | CD86 | CD79b |
| TTP | ICOS | OX40 | CD8 |
| TTP | ICOS | OX40 | CD3ζ |
| TTP | ICOS | OX40 | CD3δ |
| TTP | ICOS | OX40 | CD3γ |
| TTP | ICOS | OX40 | CD3ε |
| TTP | ICOS | OX40 | FcγRI-γ |
| TTP | ICOS | OX40 | FcγRIII-γ |
| TTP | ICOS | OX40 | FcεRIβ |
| TTP | ICOS | OX40 | FcεRIγ |
| TTP | ICOS | OX40 | DAP10 |
| TTP | ICOS | OX40 | DAP12 |
| TTP | ICOS | OX40 | CD32 |
| TTP | ICOS | OX40 | CD79a |
| TTP | ICOS | OX40 | CD79b |
| TTP | ICOS | DAP10 | CD8 |
| TTP | ICOS | DAP10 | CD3ζ |
| TTP | ICOS | DAP10 | CD3δ |
| TTP | ICOS | DAP10 | CD3γ |
| TTP | ICOS | DAP10 | CD3ε |
| TTP | ICOS | DAP10 | FcγRI-γ |
| TTP | ICOS | DAP10 | FcγRIII-γ |
| TTP | ICOS | DAP10 | FcεRIβ |
| TTP | ICOS | DAP10 | FcεRIγ |
| TTP | ICOS | DAP10 | DAP10 |
| TTP | ICOS | DAP10 | DAP12 |
| TTP | ICOS | DAP10 | CD32 |
| TTP | ICOS | DAP10 | CD79a |
| TTP | ICOS | DAP10 | CD79b |
| TTP | ICOS | DAP12 | CD8 |
| TTP | ICOS | DAP12 | CD3ζ |
| TTP | ICOS | DAP12 | CD3δ |
| TTP | ICOS | DAP12 | CD3γ |
| TTP | ICOS | DAP12 | CD3ε |
| TTP | ICOS | DAP12 | FcγRI-γ |
| TTP | ICOS | DAP12 | FcγRIII-γ |
| TTP | ICOS | DAP12 | FcεRIβ |
| TTP | ICOS | DAP12 | FcεRIγ |
| TTP | ICOS | DAP12 | DAP10 |
| TTP | ICOS | DAP12 | DAP12 |
| TTP | ICOS | DAP12 | CD32 |
| TTP | ICOS | DAP12 | CD79a |
| TTP | ICOS | DAP12 | CD79b |
| TTP | ICOS | MyD88 | CD8 |
| TTP | ICOS | MyD88 | CD3ζ |
| TTP | ICOS | MyD88 | CD3δ |
| TTP | ICOS | MyD88 | CD3γ |
| TTP | ICOS | MyD88 | CD3ε |
| TTP | ICOS | MyD88 | FcγRI-γ |
| TTP | ICOS | MyD88 | FcγRIII-γ |
| TTP | ICOS | MyD88 | FcεRIβ |
| TTP | ICOS | MyD88 | FcεRIγ |
| TTP | ICOS | MyD88 | DAP10 |
| TTP | ICOS | MyD88 | DAP12 |
| TTP | ICOS | MyD88 | CD32 |
| TTP | ICOS | MyD88 | CD79a |
| TTP | ICOS | MyD88 | CD79b |
| TTP | ICOS | CD7 | CD8 |
| TTP | ICOS | CD7 | CD3ζ |
| TTP | ICOS | CD7 | CD3δ |
| TTP | ICOS | CD7 | CD3γ |
| TTP | ICOS | CD7 | CD3ε |
| TTP | ICOS | CD7 | FcγRI-γ |
| TTP | ICOS | CD7 | FcγRIII-γ |
| TTP | ICOS | CD7 | FcεRIβ |
| TTP | ICOS | CD7 | FcεRIγ |
| TTP | ICOS | CD7 | DAP10 |
| TTP | ICOS | CD7 | DAP12 |
| TTP | ICOS | CD7 | CD32 |
| TTP | ICOS | CD7 | CD79a |
| TTP | ICOS | CD7 | CD79b |
| TTP | ICOS | BTNL3 | CD8 |
| TTP | ICOS | BTNL3 | CD3ζ |
| TTP | ICOS | BTNL3 | CD3δ |
| TTP | ICOS | BTNL3 | CD3γ |
| TTP | ICOS | BTNL3 | CD3ε |
| TTP | ICOS | BTNL3 | FcγRI-γ |
| TTP | ICOS | BTNL3 | FcγRIII-γ |
| TTP | ICOS | BTNL3 | FcεRIβ |
| TTP | ICOS | BTNL3 | FcεRIγ |
| TTP | ICOS | BTNL3 | DAP10 |
| TTP | ICOS | BTNL3 | DAP12 |
| TTP | ICOS | BTNL3 | CD32 |
| TTP | ICOS | BTNL3 | CD79a |
| TTP | ICOS | BTNL3 | CD79b |
| TTP | ICOS | NKG2D | CD8 |
| TTP | ICOS | NKG2D | CD3ζ |
| TTP | ICOS | NKG2D | CD3δ |
| TTP | ICOS | NKG2D | CD3γ |
| TTP | ICOS | NKG2D | CD3ε |
| TTP | ICOS | NKG2D | FcγRI-γ |
| TTP | ICOS | NKG2D | FcγRIII-γ |
| TTP | ICOS | NKG2D | FcεRIβ |
| TTP | ICOS | NKG2D | FcεRIγ |
| TTP | ICOS | NKG2D | DAP10 |
| TTP | ICOS | NKG2D | DAP12 |
| TTP | ICOS | NKG2D | CD32 |
| TTP | ICOS | NKG2D | CD79a |
| TTP | ICOS | NKG2D | CD79b |
| TTP | CD27 | CD28 | CD8 |
| TTP | CD27 | CD28 | CD3ζ |
| TTP | CD27 | CD28 | CD3δ |
| TTP | CD27 | CD28 | CD3γ |
| TTP | CD27 | CD28 | CD3ε |
| TTP | CD27 | CD28 | FcγRI-γ |
| TTP | CD27 | CD28 | FcγRIII-γ |
| TTP | CD27 | CD28 | FcεRIβ |
| TTP | CD27 | CD28 | FcεRIγ |
| TTP | CD27 | CD28 | DAP10 |
| TTP | CD27 | CD28 | DAP12 |
| TTP | CD27 | CD28 | CD32 |
| TTP | CD27 | CD28 | CD79a |
| TTP | CD27 | CD28 | CD79b |
| TTP | CD27 | CD8 | CD8 |
| TTP | CD27 | CD8 | CD3ζ |
| TTP | CD27 | CD8 | CD3δ |
| TTP | CD27 | CD8 | CD3γ |
| TTP | CD27 | CD8 | CD3ε |
| TTP | CD27 | CD8 | FcγRI-γ |
| TTP | CD27 | CD8 | FcγRIII-γ |
| TTP | CD27 | CD8 | FcεRIβ |
| TTP | CD27 | CD8 | FcεRIγ |
| TTP | CD27 | CD8 | DAP10 |
| TTP | CD27 | CD8 | DAP12 |
| TTP | CD27 | CD8 | CD32 |
| TTP | CD27 | CD8 | CD79a |
| TTP | CD27 | CD8 | CD79b |
| TTP | CD27 | CD4 | CD8 |
| TTP | CD27 | CD4 | CD3ζ |
| TTP | CD27 | CD4 | CD3δ |
| TTP | CD27 | CD4 | CD3γ |
| TTP | CD27 | CD4 | CD3ε |
| TTP | CD27 | CD4 | FcγRI-γ |
| TTP | CD27 | CD4 | FcγRIII-γ |
| TTP | CD27 | CD4 | FcεRIβ |
| TTP | CD27 | CD4 | FcεRIγ |
| TTP | CD27 | CD4 | DAP10 |
| TTP | CD27 | CD4 | DAP12 |
| TTP | CD27 | CD4 | CD32 |
| TTP | CD27 | CD4 | CD79a |
| TTP | CD27 | CD4 | CD79b |
| TTP | CD27 | b2c | CD8 |
| TTP | CD27 | b2c | CD3ζ |
| TTP | CD27 | b2c | CD3δ |
| TTP | CD27 | b2c | CD3γ |
| TTP | CD27 | b2c | CD3ε |
| TTP | CD27 | b2c | FcγRI-γ |
| TTP | CD27 | b2c | FcγRIII-γ |
| TTP | CD27 | b2c | FcεRIβ |
| TTP | CD27 | b2c | FcεRIγ |
| TTP | CD27 | b2c | DAP10 |
| TTP | CD27 | b2c | DAP12 |
| TTP | CD27 | b2c | CD32 |
| TTP | CD27 | b2c | CD79a |
| TTP | CD27 | b2c | CD79b |
| TTP | CD27 | CD137/41BB | CD8 |
| TTP | CD27 | CD137/41BB | CD3ζ |
| TTP | CD27 | CD137/41BB | CD3δ |
| TTP | CD27 | CD137/41BB | CD3γ |
| TTP | CD27 | CD137/41BB | CD3ε |
| TTP | CD27 | CD137/41BB | FcγRI-γ |
| TTP | CD27 | CD137/41BB | FcγRIII-γ |
| TTP | CD27 | CD137/41BB | FcεRIβ |
| TTP | CD27 | CD137/41BB | FcεRIγ |
| TTP | CD27 | CD137/41BB | DAP10 |
| TTP | CD27 | CD137/41BB | DAP12 |
| TTP | CD27 | CD137/41BB | CD32 |
| TTP | CD27 | CD137/41BB | CD79a |
| TTP | CD27 | CD137/41BB | CD79b |
| TTP | CD27 | ICOS | CD8 |
| TTP | CD27 | ICOS | CD3ζ |
| TTP | CD27 | ICOS | CD3δ |
| TTP | CD27 | ICOS | CD3γ |
| TTP | CD27 | ICOS | CD3ε |
| TTP | CD27 | ICOS | FcγRI-γ |
| TTP | CD27 | ICOS | FcγRIII-y |
| TTP | CD27 | ICOS | FcεRIβ |
| TTP | CD27 | ICOS | FcεRIγ |
| TTP | CD27 | ICOS | DAP10 |
| TTP | CD27 | ICOS | DAP12 |
| TTP | CD27 | ICOS | CD32 |
| TTP | CD27 | ICOS | CD79a |
| TTP | CD27 | ICOS | CD79b |
| TTP | CD27 | CD27 | CD8 |
| TTP | CD27 | CD27 | CD3ζ |
| TTP | CD27 | CD27 | CD3δ |
| TTP | CD27 | CD27 | CD3γ |
| TTP | CD27 | CD27 | CD3ε |
| TTP | CD27 | CD27 | FcγRI-γ |
| TTP | CD27 | CD27 | FcγRIII-γ |
| TTP | CD27 | CD27 | FcεRIβ |
| TTP | CD27 | CD27 | FcεRIγ |
| TTP | CD27 | CD27 | DAP10 |
| TTP | CD27 | CD27 | DAP12 |
| TTP | CD27 | CD27 | CD32 |
| TTP | CD27 | CD27 | CD79a |
| TTP | CD27 | CD27 | CD79b |
| TTP | CD27 | CD28δ | CD8 |
| TTP | CD27 | CD28δ | CD3ζ |
| TTP | CD27 | CD28δ | CD3δ |
| TTP | CD27 | CD28δ | CD3γ |
| TTP | CD27 | CD28δ | CD3ε |
| TTP | CD27 | CD28δ | FcγRI-γ |
| TTP | CD27 | CD28δ | FcγRIII-γ |
| TTP | CD27 | CD28δ | FcεRIβ |
| TTP | CD27 | CD28δ | FcεRIγ |
| TTP | CD27 | CD28δ | DAP10 |
| TTP | CD27 | CD28δ | DAP12 |
| TTP | CD27 | CD28δ | CD32 |
| TTP | CD27 | CD28δ | CD79a |
| TTP | CD27 | CD28δ | CD79b |
| TTP | CD27 | CD80 | CD8 |
| TTP | CD27 | CD80 | CD3ζ |
| TTP | CD27 | CD80 | CD3δ |
| TTP | CD27 | CD80 | CD3γ |
| TTP | CD27 | CD80 | CD3ε |
| TTP | CD27 | CD80 | FcγRI-γ |
| TTP | CD27 | CD80 | FcγRIII-γ |
| TTP | CD27 | CD80 | FcεRIβ |
| TTP | CD27 | CD80 | FcεRIγ |
| TTP | CD27 | CD80 | DAP10 |
| TTP | CD27 | CD80 | DAP12 |
| TTP | CD27 | CD80 | CD32 |
| TTP | CD27 | CD80 | CD79a |
| TTP | CD27 | CD80 | CD79b |
| TTP | CD27 | CD86 | CD8 |
| TTP | CD27 | CD86 | CD3ζ |
| TTP | CD27 | CD86 | CD3δ |
| TTP | CD27 | CD86 | CD3γ |
| TTP | CD27 | CD86 | CD3ε |
| TTP | CD27 | CD86 | FcγRI-γ |
| TTP | CD27 | CD86 | FcγRIII-γ |
| TTP | CD27 | CD86 | FcεRIβ |
| TTP | CD27 | CD86 | FcεRIγ |
| TTP | CD27 | CD86 | DAP10 |
| TTP | CD27 | CD86 | DAP12 |
| TTP | CD27 | CD86 | CD32 |
| TTP | CD27 | CD86 | CD79a |
| TTP | CD27 | CD86 | CD79b |
| TTP | CD27 | OX40 | CD8 |
| TTP | CD27 | OX40 | CD3ζ |
| TTP | CD27 | OX40 | CD3δ |
| TTP | CD27 | OX40 | CD3γ |
| TTP | CD27 | OX40 | CD3ε |
| TTP | CD27 | OX40 | FcγRI-γ |
| TTP | CD27 | OX40 | FcγRIII-γ |
| TTP | CD27 | OX40 | FcεRIβ |
| TTP | CD27 | OX40 | FcεRIγ |
| TTP | CD27 | OX40 | DAP10 |
| TTP | CD27 | OX40 | DAP12 |
| TTP | CD27 | OX40 | CD32 |
| TTP | CD27 | OX40 | CD79a |
| TTP | CD27 | OX40 | CD79b |
| TTP | CD27 | DAP10 | CD8 |
| TTP | CD27 | DAP10 | CD3ζ |
| TTP | CD27 | DAP10 | CD3δ |
| TTP | CD27 | DAP10 | CD3γ |
| TTP | CD27 | DAP10 | CD3ε |
| TTP | CD27 | DAP10 | FcγRI-γ |
| TTP | CD27 | DAP10 | FcγRIII-γ |
| TTP | CD27 | DAP10 | FcεRIβ |
| TTP | CD27 | DAP10 | FcεRIγ |
| TTP | CD27 | DAP10 | DAP10 |
| TTP | CD27 | DAP10 | DAP12 |
| TTP | CD27 | DAP10 | CD32 |
| TTP | CD27 | DAP10 | CD79a |
| TTP | CD27 | DAP10 | CD79b |
| TTP | CD27 | DAP12 | CD8 |
| TTP | CD27 | DAP12 | CD3ζ |
| TTP | CD27 | DAP12 | CD3δ |
| TTP | CD27 | DAP12 | CD3γ |
| TTP | CD27 | DAP12 | CD3ε |
| TTP | CD27 | DAP12 | FcγRI-γ |
| TTP | CD27 | DAP12 | FcγRIII-γ |
| TTP | CD27 | DAP12 | FcεRIβ |
| TTP | CD27 | DAP12 | FcεRIγ |
| TTP | CD27 | DAP12 | DAP10 |
| TTP | CD27 | DAP12 | DAP12 |
| TTP | CD27 | DAP12 | CD32 |
| TTP | CD27 | DAP12 | CD79a |
| TTP | CD27 | DAP12 | CD79b |
| TTP | CD27 | MyD88 | CD8 |
| TTP | CD27 | MyD88 | CD3ζ |
| TTP | CD27 | MyD88 | CD3δ |
| TTP | CD27 | MyD88 | CD3γ |
| TTP | CD27 | MyD88 | CD3ε |
| TTP | CD27 | MyD88 | FcγRI-γ |
| TTP | CD27 | MyD88 | FcγRIII-γ |
| TTP | CD27 | MyD88 | FcεRIβ |
| TTP | CD27 | MyD88 | FcεRIγ |
| TTP | CD27 | MyD88 | DAP10 |
| TTP | CD27 | MyD88 | DAP12 |
| TTP | CD27 | MyD88 | CD32 |
| TTP | CD27 | MyD88 | CD79a |
| TTP | CD27 | MyD88 | CD79b |
| TTP | CD27 | CD7 | CD8 |
| TTP | CD27 | CD7 | CD3ζ |
| TTP | CD27 | CD7 | CD3δ |
| TTP | CD27 | CD7 | CD3γ |
| TTP | CD27 | CD7 | CD3ε |
| TTP | CD27 | CD7 | FcγRI-γ |
| TTP | CD27 | CD7 | FcγRIII-γ |
| TTP | CD27 | CD7 | FcεRIβ |
| TTP | CD27 | CD7 | FcεRIγ |
| TTP | CD27 | CD7 | DAP10 |
| TTP | CD27 | CD7 | DAP12 |
| TTP | CD27 | CD7 | CD32 |
| TTP | CD27 | CD7 | CD79a |
| TTP | CD27 | CD7 | CD79b |
| TTP | CD27 | BTNL3 | CD8 |
| TTP | CD27 | BTNL3 | CD3ζ |
| TTP | CD27 | BTNL3 | CD3δ |
| TTP | CD27 | BTNL3 | CD3γ |
| TTP | CD27 | BTNL3 | CD3ε |
| TTP | CD27 | BTNL3 | FcγRI-γ |
| TTP | CD27 | BTNL3 | FcγRIII-γ |
| TTP | CD27 | BTNL3 | FcεRIβ |
| TTP | CD27 | BTNL3 | FcεRIγ |
| TTP | CD27 | BTNL3 | DAP10 |
| TTP | CD27 | BTNL3 | DAP12 |
| TTP | CD27 | BTNL3 | CD32 |
| TTP | CD27 | BTNL3 | CD79a |
| TTP | CD27 | BTNL3 | CD79b |
| TTP | CD27 | NKG2D | CD8 |
| TTP | CD27 | NKG2D | CD3ζ |
| TTP | CD27 | NKG2D | CD3δ |
| TTP | CD27 | NKG2D | CD3γ |
| TTP | CD27 | NKG2D | CD3ε |
| TTP | CD27 | NKG2D | FcγRI-γ |
| TTP | CD27 | NKG2D | FcγRIII-γ |
| TTP | CD27 | NKG2D | FcεRIβ |
| TTP | CD27 | NKG2D | FcεRIγ |
| TTP | CD27 | NKG2D | DAP10 |
| TTP | CD27 | NKG2D | DAP12 |
| TTP | CD27 | NKG2D | CD32 |
| TTP | CD27 | NKG2D | CD79a |
| TTP | CD27 | NKG2D | CD79b |
| TTP | CD28δ | CD28 | CD8 |
| TTP | CD28δ | CD28 | CD3ζ |
| TTP | CD28δ | CD28 | CD3δ |
| TTP | CD28δ | CD28 | CD3γ |
| TTP | CD28δ | CD28 | CD3ε |
| TTP | CD28δ | CD28 | FcγRI-γ |
| TTP | CD28δ | CD28 | FcγRIII-γ |
| TTP | CD28δ | CD28 | FcεRIβ |
| TTP | CD28δ | CD28 | FcεRIγ |
| TTP | CD28δ | CD28 | DAP10 |
| TTP | CD28δ | CD28 | DAP12 |
| TTP | CD28δ | CD28 | CD32 |
| TTP | CD28δ | CD28 | CD79a |
| TTP | CD28δ | CD28 | CD79b |
| TTP | CD28δ | CD8 | CD8 |
| TTP | CD28δ | CD8 | CD3ζ |
| TTP | CD28δ | CD8 | CD3δ |
| TTP | CD28δ | CD8 | CD3γ |
| TTP | CD28δ | CD8 | CD3ε |
| TTP | CD28δ | CD8 | FcγRI-γ |
| TTP | CD28δ | CD8 | FcγRIII-γ |
| TTP | CD28δ | CD8 | FcεRIβ |
| TTP | CD28δ | CD8 | FcεRIγ |
| TTP | CD28δ | CD8 | DAP10 |
| TTP | CD28δ | CD8 | DAP12 |
| TTP | CD28δ | CD8 | CD32 |
| TTP | CD28δ | CD8 | CD79a |
| TTP | CD28δ | CD8 | CD79b |
| TTP | CD28δ | CD4 | CD8 |
| TTP | CD28δ | CD4 | CD3ζ |
| TTP | CD28δ | CD4 | CD3δ |
| TTP | CD28δ | CD4 | CD3γ |
| TTP | CD28δ | CD4 | CD3ε |
| TTP | CD28δ | CD4 | FcγRI-γ |
| TTP | CD28δ | CD4 | FcγRIII-γ |
| TTP | CD28δ | CD4 | FcεRIβ |
| TTP | CD28δ | CD4 | FcεRIγ |
| TTP | CD28δ | CD4 | DAP10 |
| TTP | CD28δ | CD4 | DAP12 |
| TTP | CD28δ | CD4 | CD32 |
| TTP | CD28δ | CD4 | CD79a |
| TTP | CD28δ | CD4 | CD79b |
| TTP | CD28δ | b2c | CD8 |
| TTP | CD28δ | b2c | CD3ζ |
| TTP | CD28δ | b2c | CD3δ |
| TTP | CD28δ | b2c | CD3γ |
| TTP | CD28δ | b2c | CD3ε |
| TTP | CD28δ | b2c | FcγRI-γ |
| TTP | CD28δ | b2c | FcγRIII-γ |
| TTP | CD28δ | b2c | FcεRIβ |
| TTP | CD28δ | b2c | FcεRIγ |
| TTP | CD28δ | b2c | DAP10 |
| TTP | CD28δ | b2c | DAP12 |
| TTP | CD28δ | b2c | CD32 |
| TTP | CD28δ | b2c | CD79a |
| TTP | CD28δ | b2c | CD79b |
| TTP | CD28δ | CD137/41BB | CD8 |
| TTP | CD28δ | CD137/41BB | CD3ζ |
| TTP | CD28δ | CD137/41BB | CD3δ |
| TTP | CD28δ | CD137/41BB | CD3γ |
| TTP | CD28δ | CD137/41BB | CD3ε |
| TTP | CD28δ | CD137/41BB | FcγRI-γ |
| TTP | CD28δ | CD137/41BB | FcγRIII-γ |
| TTP | CD28δ | CD137/41BB | FcεRIβ |
| TTP | CD28δ | CD137/41BB | FcεRIγ |
| TTP | CD28δ | CD137/41BB | DAP10 |
| TTP | CD28δ | CD137/41BB | DAP12 |
| TTP | CD28δ | CD137/41BB | CD32 |
| TTP | CD28δ | CD137/41BB | CD79a |
| TTP | CD28δ | CD137/41BB | CD79b |
| TTP | CD28δ | ICOS | CD8 |
| TTP | CD28δ | ICOS | CD3ζ |
| TTP | CD28δ | ICOS | CD3δ |
| TTP | CD28δ | ICOS | CD3γ |
| TTP | CD28δ | ICOS | CD3ε |
| TTP | CD28δ | ICOS | FcγRI-γ |
| TTP | CD28δ | ICOS | FcγRIII-γ |
| TTP | CD28δ | ICOS | FcεRIβ |
| TTP | CD28δ | ICOS | FcεRIγ |
| TTP | CD28δ | ICOS | DAP10 |
| TTP | CD28δ | ICOS | DAP12 |
| TTP | CD28δ | ICOS | CD32 |
| TTP | CD28δ | ICOS | CD79a |
| TTP | CD28δ | ICOS | CD79b |
| TTP | CD28δ | CD27 | CD8 |
| TTP | CD28δ | CD27 | CD3ζ |
| TTP | CD28δ | CD27 | CD3δ |
| TTP | CD28δ | CD27 | CD3γ |
| TTP | CD28δ | CD27 | CD3ε |
| TTP | CD28δ | CD27 | FcγRI-γ |
| TTP | CD28δ | CD27 | FcγRIII-γ |
| TTP | CD28δ | CD27 | FcεRIβ |
| TTP | CD28δ | CD27 | FcεRIγ |
| TTP | CD28δ | CD27 | DAP10 |
| TTP | CD28δ | CD27 | DAP12 |
| TTP | CD28δ | CD27 | CD32 |
| TTP | CD28δ | CD27 | CD79a |
| TTP | CD28δ | CD27 | CD79b |
| TTP | CD28δ | CD28δ | CD8 |
| TTP | CD28δ | CD28δ | CD3ζ |
| TTP | CD28δ | CD28δ | CD3δ |
| TTP | CD28δ | CD28δ | CD3γ |
| TTP | CD28δ | CD28δ | CD3ε |
| TTP | CD28δ | CD28δ | FcγRI-γ |
| TTP | CD28δ | CD28δ | FcγRIII-γ |
| TTP | CD28δ | CD28δ | FcεRIβ |
| TTP | CD28δ | CD28δ | FcεRIγ |
| TTP | CD28δ | CD28δ | DAP10 |
| TTP | CD28δ | CD28δ | DAP12 |
| TTP | CD28δ | CD28δ | CD32 |
| TTP | CD28δ | CD28δ | CD79a |
| TTP | CD28δ | CD28δ | CD79b |
| TTP | CD28δ | CD80 | CD8 |
| TTP | CD28δ | CD80 | CD3ζ |
| TTP | CD28δ | CD80 | CD3δ |
| TTP | CD28δ | CD80 | CD3γ |
| TTP | CD28δ | CD80 | CD3ε |
| TTP | CD28δ | CD80 | FcγRI-γ |
| TTP | CD28δ | CD80 | FcγRIII-γ |
| TTP | CD28δ | CD80 | FcεRIβ |
| TTP | CD28δ | CD80 | FcεRIγ |
| TTP | CD28δ | CD80 | DAP10 |
| TTP | CD28δ | CD80 | DAP12 |
| TTP | CD28δ | CD80 | CD32 |
| TTP | CD28δ | CD80 | CD79a |
| TTP | CD28δ | CD80 | CD79b |
| TTP | CD28δ | CD86 | CD8 |
| TTP | CD28δ | CD86 | CD3ζ |
| TTP | CD28δ | CD86 | CD3δ |
| TTP | CD28δ | CD86 | CD3γ |
| TTP | CD28δ | CD86 | CD3ε |
| TTP | CD28δ | CD86 | FcγRI-γ |
| TTP | CD28δ | CD86 | FcγRIII-γ |
| TTP | CD28δ | CD86 | FcεRIβ |
| TTP | CD28δ | CD86 | FcεRIγ |
| TTP | CD28δ | CD86 | DAP10 |
| TTP | CD28δ | CD86 | DAP12 |
| TTP | CD28δ | CD86 | CD32 |
| TTP | CD28δ | CD86 | CD79a |
| TTP | CD28δ | CD86 | CD79b |
| TTP | CD28δ | OX40 | CD8 |
| TTP | CD28δ | OX40 | CD3ζ |
| TTP | CD28δ | OX40 | CD3δ |
| TTP | CD28δ | OX40 | CD3γ |
| TTP | CD28δ | OX40 | CD3ε |
| TTP | CD28δ | OX40 | FcγRI-γ |
| TTP | CD28δ | OX40 | FcγRIII-γ |
| TTP | CD28δ | OX40 | FcεRIβ |
| TTP | CD28δ | OX40 | FcεRIγ |
| TTP | CD28δ | OX40 | DAP10 |
| TTP | CD28δ | OX40 | DAP12 |
| TTP | CD28δ | OX40 | CD32 |
| TTP | CD28δ | OX40 | CD79a |
| TTP | CD28δ | OX40 | CD79b |
| TTP | CD28δ | DAP10 | CD8 |
| TTP | CD28δ | DAP10 | CD3ζ |
| TTP | CD28δ | DAP10 | CD3δ |
| TTP | CD28δ | DAP10 | CD3γ |
| TTP | CD28δ | DAP10 | CD3ε |
| TTP | CD28δ | DAP10 | FcγRI-γ |
| TTP | CD28δ | DAP10 | FcγRIII-γ |
| TTP | CD28δ | DAP10 | FcεRIβ |
| TTP | CD28δ | DAP10 | FcεRIγ |
| TTP | CD28δ | DAP10 | DAP10 |
| TTP | CD28δ | DAP10 | DAP12 |
| TTP | CD28δ | DAP10 | CD32 |
| TTP | CD28δ | DAP10 | CD79a |
| TTP | CD28δ | DAP10 | CD79b |
| TTP | CD28δ | DAP12 | CD8 |
| TTP | CD28δ | DAP12 | CD3ζ |
| TTP | CD28δ | DAP12 | CD3δ |
| TTP | CD28δ | DAP12 | CD3γ |
| TTP | CD28δ | DAP12 | CD3ε |
| TTP | CD28δ | DAP12 | FcγRI-γ |
| TTP | CD28δ | DAP12 | FcγRIII-γ |
| TTP | CD28δ | DAP12 | FcεRIβ |
| TTP | CD28δ | DAP12 | FcεRIγ |
| TTP | CD28δ | DAP12 | DAP10 |
| TTP | CD28δ | DAP12 | DAP12 |
| TTP | CD28δ | DAP12 | CD32 |
| TTP | CD28δ | DAP12 | CD79a |
| TTP | CD28δ | DAP12 | CD79b |
| TTP | CD28δ | MyD88 | CD8 |
| TTP | CD28δ | MyD88 | CD3ζ |
| TTP | CD28δ | MyD88 | CD3δ |
| TTP | CD28δ | MyD88 | CD3γ |
| TTP | CD28δ | MyD88 | CD3ε |
| TTP | CD28δ | MyD88 | FcγRI-γ |
| TTP | CD28δ | MyD88 | FcγRIII-γ |
| TTP | CD28δ | MyD88 | FcεRIβ |
| TTP | CD28δ | MyD88 | FcεRIγ |
| TTP | CD28δ | MyD88 | DAP10 |
| TTP | CD28δ | MyD88 | DAP12 |
| TTP | CD28δ | MyD88 | CD32 |
| TTP | CD28δ | MyD88 | CD79a |
| TTP | CD28δ | MyD88 | CD79b |
| TTP | CD28δ | CD7 | CD8 |
| TTP | CD28δ | CD7 | CD3ζ |
| TTP | CD28δ | CD7 | CD3δ |
| TTP | CD28δ | CD7 | CD3γ |
| TTP | CD28δ | CD7 | CD3ε |
| TTP | CD28δ | CD7 | FcγRI-γ |
| TTP | CD28δ | CD7 | FcγRIII-γ |
| TTP | CD28δ | CD7 | FcεRIβ |
| TTP | CD28δ | CD7 | FcεRIγ |
| TTP | CD28δ | CD7 | DAP10 |
| TTP | CD28δ | CD7 | DAP12 |
| TTP | CD28δ | CD7 | CD32 |
| TTP | CD28δ | CD7 | CD79a |
| TTP | CD28δ | CD7 | CD79b |
| TTP | CD28δ | BTNL3 | CD8 |
| TTP | CD28δ | BTNL3 | CD3ζ |
| TTP | CD28δ | BTNL3 | CD3δ |
| TTP | CD28δ | BTNL3 | CD3γ |
| TTP | CD28δ | BTNL3 | CD3ε |
| TTP | CD28δ | BTNL3 | FcγRI-γ |
| TTP | CD28δ | BTNL3 | FcγRIII-γ |
| TTP | CD28δ | BTNL3 | FcεRIβ |
| TTP | CD28δ | BTNL3 | FcεRIγ |
| TTP | CD28δ | BTNL3 | DAP10 |
| TTP | CD28δ | BTNL3 | DAP12 |
| TTP | CD28δ | BTNL3 | CD32 |
| TTP | CD28δ | BTNL3 | CD79a |
| TTP | CD28δ | BTNL3 | CD79b |
| TTP | CD28δ | NKG2D | CD8 |
| TTP | CD28δ | NKG2D | CD3ζ |
| TTP | CD28δ | NKG2D | CD3δ |
| TTP | CD28δ | NKG2D | CD3γ |
| TTP | CD28δ | NKG2D | CD3ε |
| TTP | CD28δ | NKG2D | FcγRI-γ |
| TTP | CD28δ | NKG2D | FcγRIII-γ |
| TTP | CD28δ | NKG2D | FcεRIβ |
| TTP | CD28δ | NKG2D | FcεRIγ |
| TTP | CD28δ | NKG2D | DAP10 |
| TTP | CD28δ | NKG2D | DAP12 |
| TTP | CD28δ | NKG2D | CD32 |
| TTP | CD28δ | NKG2D | CD79a |
| TTP | CD28δ | NKG2D | CD79b |
| TTP | CD80 | CD28 | CD8 |
| TTP | CD80 | CD28 | CD3ζ |
| TTP | CD80 | CD28 | CD3δ |
| TTP | CD80 | CD28 | CD3γ |
| TTP | CD80 | CD28 | CD3ε |
| TTP | CD80 | CD28 | FcγRI-γ |
| TTP | CD80 | CD28 | FcγRIII-γ |
| TTP | CD80 | CD28 | FcεRIβ |
| TTP | CD80 | CD28 | FcεRIγ |
| TTP | CD80 | CD28 | DAP10 |
| TTP | CD80 | CD28 | DAP12 |
| TTP | CD80 | CD28 | CD32 |
| TTP | CD80 | CD28 | CD79a |
| TTP | CD80 | CD28 | CD79b |
| TTP | CD80 | CD8 | CD8 |
| TTP | CD80 | CD8 | CD3ζ |
| TTP | CD80 | CD8 | CD3δ |
| TTP | CD80 | CD8 | CD3γ |
| TTP | CD80 | CD8 | CD3ε |
| TTP | CD80 | CD8 | FcγRI-γ |
| TTP | CD80 | CD8 | FcγRIII-γ |
| TTP | CD80 | CD8 | FcεRIβ |
| TTP | CD80 | CD8 | FcεRIγ |
| TTP | CD80 | CD8 | DAP10 |
| TTP | CD80 | CD8 | DAP12 |
| TTP | CD80 | CD8 | CD32 |
| TTP | CD80 | CD8 | CD79a |
| TTP | CD80 | CD8 | CD79b |
| TTP | CD80 | CD4 | CD8 |
| TTP | CD80 | CD4 | CD3ζ |
| TTP | CD80 | CD4 | CD3δ |
| TTP | CD80 | CD4 | CD3γ |
| TTP | CD80 | CD4 | CD3ε |
| TTP | CD80 | CD4 | FcγRI-γ |
| TTP | CD80 | CD4 | FcγRIII-γ |
| TTP | CD80 | CD4 | FcεRIβ |
| TTP | CD80 | CD4 | FcεRIγ |
| TTP | CD80 | CD4 | DAP10 |
| TTP | CD80 | CD4 | DAP12 |
| TTP | CD80 | CD4 | CD32 |
| TTP | CD80 | CD4 | CD79a |
| TTP | CD80 | CD4 | CD79b |
| TTP | CD80 | b2c | CD8 |
| TTP | CD80 | b2c | CD3ζ |
| TTP | CD80 | b2c | CD3δ |
| TTP | CD80 | b2c | CD3γ |
| TTP | CD80 | b2c | CD3ε |
| TTP | CD80 | b2c | FcγRI-γ |
| TTP | CD80 | b2c | FcγRIII-γ |
| TTP | CD80 | b2c | FcεRIβ |
| TTP | CD80 | b2c | FcεRIγ |
| TTP | CD80 | b2c | DAP10 |
| TTP | CD80 | b2c | DAP12 |
| TTP | CD80 | b2c | CD32 |
| TTP | CD80 | b2c | CD79a |
| TTP | CD80 | b2c | CD79b |
| TTP | CD80 | CD137/41BB | CD8 |
| TTP | CD80 | CD137/41BB | CD3ζ |
| TTP | CD80 | CD137/41BB | CD3δ |
| TTP | CD80 | CD137/41BB | CD3γ |
| TTP | CD80 | CD137/41BB | CD3ε |
| TTP | CD80 | CD137/41BB | FcγRI-γ |
| TTP | CD80 | CD137/41BB | FcγRIII-γ |
| TTP | CD80 | CD137/41BB | FcεRIβ |
| TTP | CD80 | CD137/41BB | FcεRIγ |
| TTP | CD80 | CD137/41BB | DAP10 |
| TTP | CD80 | CD137/41BB | DAP12 |
| TTP | CD80 | CD137/41BB | CD32 |
| TTP | CD80 | CD137/41BB | CD79a |
| TTP | CD80 | CD137/41BB | CD79b |
| TTP | CD80 | ICOS | CD8 |
| TTP | CD80 | ICOS | CD3ζ |
| TTP | CD80 | ICOS | CD3δ |
| TTP | CD80 | ICOS | CD3γ |
| TTP | CD80 | ICOS | CD3ε |
| TTP | CD80 | ICOS | FcγRI-γ |
| TTP | CD80 | ICOS | FcγRIII-γ |
| TTP | CD80 | ICOS | FcεRIβ |
| TTP | CD80 | ICOS | FcεRIγ |
| TTP | CD80 | ICOS | DAP10 |
| TTP | CD80 | ICOS | DAP12 |
| TTP | CD80 | ICOS | CD32 |
| TTP | CD80 | ICOS | CD79a |
| TTP | CD80 | ICOS | CD79b |
| TTP | CD80 | CD27 | CD8 |
| TTP | CD80 | CD27 | CD3ζ |
| TTP | CD80 | CD27 | CD3δ |
| TTP | CD80 | CD27 | CD3γ |
| TTP | CD80 | CD27 | CD3ε |
| TTP | CD80 | CD27 | FcγRI-γ |
| TTP | CD80 | CD27 | FcγRIII-γ |
| TTP | CD80 | CD27 | FcεRIβ |
| TTP | CD80 | CD27 | FcεRIγ |
| TTP | CD80 | CD27 | DAP10 |
| TTP | CD80 | CD27 | DAP12 |
| TTP | CD80 | CD27 | CD32 |
| TTP | CD80 | CD27 | CD79a |
| TTP | CD80 | CD27 | CD79b |
| TTP | CD80 | CD28δ | CD8 |
| TTP | CD80 | CD28δ | CD3ζ |
| TTP | CD80 | CD28δ | CD3δ |
| TTP | CD80 | CD28δ | CD3γ |
| TTP | CD80 | CD28δ | CD3ε |
| TTP | CD80 | CD28δ | FcγRI-γ |
| TTP | CD80 | CD28δ | FcγRIII-γ |
| TTP | CD80 | CD28δ | FcεRIβ |
| TTP | CD80 | CD28δ | FcεRIγ |
| TTP | CD80 | CD28δ | DAP10 |
| TTP | CD80 | CD28δ | DAP12 |
| TTP | CD80 | CD28δ | CD32 |
| TTP | CD80 | CD28δ | CD79a |
| TTP | CD80 | CD28δ | CD79b |
| TTP | CD80 | CD80 | CD8 |
| TTP | CD80 | CD80 | CD3ζ |
| TTP | CD80 | CD80 | CD3δ |
| TTP | CD80 | CD80 | CD3γ |
| TTP | CD80 | CD80 | CD3ε |
| TTP | CD80 | CD80 | FcγRI-γ |
| TTP | CD80 | CD80 | FcγRIII-γ |
| TTP | CD80 | CD80 | FcεRIβ |
| TTP | CD80 | CD80 | FcεRIγ |
| TTP | CD80 | CD80 | DAP10 |
| TTP | CD80 | CD80 | DAP12 |
| TTP | CD80 | CD80 | CD32 |
| TTP | CD80 | CD80 | CD79a |
| TTP | CD80 | CD80 | CD79b |
| TTP | CD80 | CD86 | CD8 |
| TTP | CD80 | CD86 | CD3ζ |
| TTP | CD80 | CD86 | CD3δ |
| TTP | CD80 | CD86 | CD3γ |
| TTP | CD80 | CD86 | CD3ε |
| TTP | CD80 | CD86 | FcγRI-γ |
| TTP | CD80 | CD86 | FcγRIII-γ |
| TTP | CD80 | CD86 | FcεRIβ |
| TTP | CD80 | CD86 | FcεRIγ |
| TTP | CD80 | CD86 | DAP10 |
| TTP | CD80 | CD86 | DAP12 |
| TTP | CD80 | CD86 | CD32 |
| TTP | CD80 | CD86 | CD79a |
| TTP | CD80 | CD86 | CD79b |
| TTP | CD80 | OX40 | CD8 |
| TTP | CD80 | OX40 | CD3ζ |
| TTP | CD80 | OX40 | CD3δ |
| TTP | CD80 | OX40 | CD3γ |
| TTP | CD80 | OX40 | CD3ε |
| TTP | CD80 | OX40 | FcγRI-γ |
| TTP | CD80 | OX40 | FcγRIII-γ |
| TTP | CD80 | OX40 | FcεRIβ |
| TTP | CD80 | OX40 | FcεRIγ |
| TTP | CD80 | OX40 | DAP10 |
| TTP | CD80 | OX40 | DAP12 |
| TTP | CD80 | OX40 | CD32 |
| TTP | CD80 | OX40 | CD79a |
| TTP | CD80 | OX40 | CD79b |
| TTP | CD80 | DAP10 | CD8 |
| TTP | CD80 | DAP10 | CD3ζ |
| TTP | CD80 | DAP10 | CD3δ |
| TTP | CD80 | DAP10 | CD3γ |
| TTP | CD80 | DAP10 | CD3ε |
| TTP | CD80 | DAP10 | FcγRI-γ |
| TTP | CD80 | DAP10 | FcγRIII-γ |
| TTP | CD80 | DAP10 | FcεRIβ |
| TTP | CD80 | DAP10 | FcεRIγ |
| TTP | CD80 | DAP10 | DAP10 |
| TTP | CD80 | DAP10 | DAP12 |
| TTP | CD80 | DAP10 | CD32 |
| TTP | CD80 | DAP10 | CD79a |
| TTP | CD80 | DAP10 | CD79b |
| TTP | CD80 | DAP12 | CD8 |
| TTP | CD80 | DAP12 | CD3ζ |
| TTP | CD80 | DAP12 | CD3δ |
| TTP | CD80 | DAP12 | CD3γ |
| TTP | CD80 | DAP12 | CD3ε |
| TTP | CD80 | DAP12 | FcγRI-γ |
| TTP | CD80 | DAP12 | FcγRIII-γ |
| TTP | CD80 | DAP12 | FcεRIβ |
| TTP | CD80 | DAP12 | FcεRIγ |
| TTP | CD80 | DAP12 | DAP10 |
| TTP | CD80 | DAP12 | DAP12 |
| TTP | CD80 | DAP12 | CD32 |
| TTP | CD80 | DAP12 | CD79a |
| TTP | CD80 | DAP12 | CD79b |
| TTP | CD80 | MyD88 | CD8 |
| TTP | CD80 | MyD88 | CD3ζ |
| TTP | CD80 | MyD88 | CD3δ |
| TTP | CD80 | MyD88 | CD3γ |
| TTP | CD80 | MyD88 | CD3ε |
| TTP | CD80 | MyD88 | FcγRI-γ |
| TTP | CD80 | MyD88 | FcγRIII-γ |
| TTP | CD80 | MyD88 | FcεRIβ |
| TTP | CD80 | MyD88 | FcεRIγ |
| TTP | CD80 | MyD88 | DAP10 |
| TTP | CD80 | MyD88 | DAP12 |
| TTP | CD80 | MyD88 | CD32 |
| TTP | CD80 | MyD88 | CD79a |
| TTP | CD80 | MyD88 | CD79b |
| TTP | CD80 | CD7 | CD8 |
| TTP | CD80 | CD7 | CD3ζ |
| TTP | CD80 | CD7 | CD3δ |
| TTP | CD80 | CD7 | CD3γ |
| TTP | CD80 | CD7 | CD3ε |
| TTP | CD80 | CD7 | FcγRI-γ |
| TTP | CD80 | CD7 | FcγRIII-γ |
| TTP | CD80 | CD7 | FcεRIβ |
| TTP | CD80 | CD7 | FcεRIγ |
| TTP | CD80 | CD7 | DAP10 |
| TTP | CD80 | CD7 | DAP12 |
| TTP | CD80 | CD7 | CD32 |
| TTP | CD80 | CD7 | CD79a |
| TTP | CD80 | CD7 | CD79b |
| TTP | CD80 | BTNL3 | CD8 |
| TTP | CD80 | BTNL3 | CD3ζ |
| TTP | CD80 | BTNL3 | CD3δ |
| TTP | CD80 | BTNL3 | CD3γ |
| TTP | CD80 | BTNL3 | CD3ε |
| TTP | CD80 | BTNL3 | FcγRI-γ |
| TTP | CD80 | BTNL3 | FcγRIII-γ |
| TTP | CD80 | BTNL3 | FcεRIβ |
| TTP | CD80 | BTNL3 | FcεRIγ |
| TTP | CD80 | BTNL3 | DAP10 |
| TTP | CD80 | BTNL3 | DAP12 |
| TTP | CD80 | BTNL3 | CD32 |
| TTP | CD80 | BTNL3 | CD79a |
| TTP | CD80 | BTNL3 | CD79b |
| TTP | CD80 | NKG2D | CD8 |
| TTP | CD80 | NKG2D | CD3ζ |
| TTP | CD80 | NKG2D | CD3δ |
| TTP | CD80 | NKG2D | CD3γ |
| TTP | CD80 | NKG2D | CD3ε |
| TTP | CD80 | NKG2D | FcγRI-γ |
| TTP | CD80 | NKG2D | FcγRIII-γ |
| TTP | CD80 | NKG2D | FcεRIβ |
| TTP | CD80 | NKG2D | FcεRIγ |
| TTP | CD80 | NKG2D | DAP10 |
| TTP | CD80 | NKG2D | DAP12 |
| TTP | CD80 | NKG2D | CD32 |
| TTP | CD80 | NKG2D | CD79a |
| TTP | CD80 | NKG2D | CD79b |
| TTP | CD86 | CD28 | CD8 |
| TTP | CD86 | CD28 | CD3ζ |
| TTP | CD86 | CD28 | CD3δ |
| TTP | CD86 | CD28 | CD3γ |
| TTP | CD86 | CD28 | CD3ε |
| TTP | CD86 | CD28 | FcγRI-γ |
| TTP | CD86 | CD28 | FcγRIII-γ |
| TTP | CD86 | CD28 | FcεRIβ |
| TTP | CD86 | CD28 | FcεRIγ |
| TTP | CD86 | CD28 | DAP10 |
| TTP | CD86 | CD28 | DAP12 |
| TTP | CD86 | CD28 | CD32 |
| TTP | CD86 | CD28 | CD79a |
| TTP | CD86 | CD28 | CD79b |
| TTP | CD86 | CD8 | CD8 |
| TTP | CD86 | CD8 | CD3ζ |
| TTP | CD86 | CD8 | CD3δ |
| TTP | CD86 | CD8 | CD3γ |
| TTP | CD86 | CD8 | CD3ε |
| TTP | CD86 | CD8 | FcγRI-γ |
| TTP | CD86 | CD8 | FcγRIII-γ |
| TTP | CD86 | CD8 | FcεRIβ |
| TTP | CD86 | CD8 | FcεRIγ |
| TTP | CD86 | CD8 | DAP10 |
| TTP | CD86 | CD8 | DAP12 |
| TTP | CD86 | CD8 | CD32 |
| TTP | CD86 | CD8 | CD79a |
| TTP | CD86 | CD8 | CD79b |
| TTP | CD86 | CD4 | CD8 |
| TTP | CD86 | CD4 | CD3ζ |
| TTP | CD86 | CD4 | CD3δ |
| TTP | CD86 | CD4 | CD3γ |
| TTP | CD86 | CD4 | CD3ε |
| TTP | CD86 | CD4 | FcγRI-γ |
| TTP | CD86 | CD4 | FcγRIII-γ |
| TTP | CD86 | CD4 | FcεRIβ |
| TTP | CD86 | CD4 | FcεRIγ |
| TTP | CD86 | CD4 | DAP10 |
| TTP | CD86 | CD4 | DAP12 |
| TTP | CD86 | CD4 | CD32 |
| TTP | CD86 | CD4 | CD79a |
| TTP | CD86 | CD4 | CD79b |
| TTP | CD86 | b2c | CD8 |
| TTP | CD86 | b2c | CD3ζ |
| TTP | CD86 | b2c | CD3δ |
| TTP | CD86 | b2c | CD3γ |
| TTP | CD86 | b2c | CD3ε |
| TTP | CD86 | b2c | FcγRI-γ |
| TTP | CD86 | b2c | FcγRIII-γ |
| TTP | CD86 | b2c | FcεRIβ |
| TTP | CD86 | b2c | FcεRIγ |
| TTP | CD86 | b2c | DAP10 |
| TTP | CD86 | b2c | DAP12 |
| TTP | CD86 | b2c | CD32 |
| TTP | CD86 | b2c | CD79a |
| TTP | CD86 | b2c | CD79b |
| TTP | CD86 | CD137/41BB | CD8 |
| TTP | CD86 | CD137/41BB | CD3ζ |
| TTP | CD86 | CD137/41BB | CD3δ |
| TTP | CD86 | CD137/41BB | CD3γ |
| TTP | CD86 | CD137/41BB | CD3ε |
| TTP | CD86 | CD137/41BB | FcγRI-γ |
| TTP | CD86 | CD137/41BB | FcγRIII-γ |
| TTP | CD86 | CD137/41BB | FcεRIβ |
| TTP | CD86 | CD137/41BB | FcεRIγ |
| TTP | CD86 | CD137/41BB | DAP10 |
| TTP | CD86 | CD137/41BB | DAP12 |
| TTP | CD86 | CD137/41BB | CD32 |
| TTP | CD86 | CD137/41BB | CD79a |
| TTP | CD86 | CD137/41BB | CD79b |
| TTP | CD86 | ICOS | CD8 |
| TTP | CD86 | ICOS | CD3ζ |
| TTP | CD86 | ICOS | CD3δ |
| TTP | CD86 | ICOS | CD3γ |
| TTP | CD86 | ICOS | CD3ε |
| TTP | CD86 | ICOS | FcγRI-γ |
| TTP | CD86 | ICOS | FcγRIII-γ |
| TTP | CD86 | ICOS | FcεRIβ |
| TTP | CD86 | ICOS | FcεRIγ |
| TTP | CD86 | ICOS | DAP10 |
| TTP | CD86 | ICOS | DAP12 |
| TTP | CD86 | ICOS | CD32 |
| TTP | CD86 | ICOS | CD79a |
| TTP | CD86 | ICOS | CD79b |
| TTP | CD86 | CD27 | CD8 |
| TTP | CD86 | CD27 | CD3ζ |
| TTP | CD86 | CD27 | CD3δ |
| TTP | CD86 | CD27 | CD3γ |
| TTP | CD86 | CD27 | CD3ε |
| TTP | CD86 | CD27 | FcγRI-γ |
| TTP | CD86 | CD27 | FcγRIII-γ |
| TTP | CD86 | CD27 | FcεRIβ |
| TTP | CD86 | CD27 | FcεRIγ |
| TTP | CD86 | CD27 | DAP10 |
| TTP | CD86 | CD27 | DAP12 |
| TTP | CD86 | CD27 | CD32 |
| TTP | CD86 | CD27 | CD79a |
| TTP | CD86 | CD27 | CD79b |
| TTP | CD86 | CD28δ | CD8 |
| TTP | CD86 | CD28δ | CD3ζ |
| TTP | CD86 | CD28δ | CD3δ |
| TTP | CD86 | CD28δ | CD3γ |
| TTP | CD86 | CD28δ | CD3ε |
| TTP | CD86 | CD28δ | FcγRI-γ |
| TTP | CD86 | CD28δ | FcγRIII-γ |
| TTP | CD86 | CD28δ | FcεRIβ |
| TTP | CD86 | CD28δ | FcεRIγ |
| TTP | CD86 | CD28δ | DAP10 |
| TTP | CD86 | CD28δ | DAP12 |
| TTP | CD86 | CD28δ | CD32 |
| TTP | CD86 | CD28δ | CD79a |
| TTP | CD86 | CD28δ | CD79b |
| TTP | CD86 | CD80 | CD8 |
| TTP | CD86 | CD80 | CD3ζ |
| TTP | CD86 | CD80 | CD3δ |
| TTP | CD86 | CD80 | CD3γ |
| TTP | CD86 | CD80 | CD3ε |
| TTP | CD86 | CD80 | FcγRI-γ |
| TTP | CD86 | CD80 | FcγRIII-γ |
| TTP | CD86 | CD80 | FcεRIβ |
| TTP | CD86 | CD80 | FcεRIγ |
| TTP | CD86 | CD80 | DAP10 |
| TTP | CD86 | CD80 | DAP12 |
| TTP | CD86 | CD80 | CD32 |
| TTP | CD86 | CD80 | CD79a |
| TTP | CD86 | CD80 | CD79b |
| TTP | CD86 | CD86 | CD8 |
| TTP | CD86 | CD86 | CD3ζ |
| TTP | CD86 | CD86 | CD3δ |
| TTP | CD86 | CD86 | CD3γ |
| TTP | CD86 | CD86 | CD3ε |
| TTP | CD86 | CD86 | FcγRI-γ |
| TTP | CD86 | CD86 | FcγRIII-γ |
| TTP | CD86 | CD86 | FcεRIβ |
| TTP | CD86 | CD86 | FcεRIγ |
| TTP | CD86 | CD86 | DAP10 |
| TTP | CD86 | CD86 | DAP12 |
| TTP | CD86 | CD86 | CD32 |
| TTP | CD86 | CD86 | CD79a |
| TTP | CD86 | CD86 | CD79b |
| TTP | CD86 | OX40 | CD8 |
| TTP | CD86 | OX40 | CD3ζ |
| TTP | CD86 | OX40 | CD3δ |
| TTP | CD86 | OX40 | CD3γ |
| TTP | CD86 | OX40 | CD3ε |
| TTP | CD86 | OX40 | FcγRI-γ |
| TTP | CD86 | OX40 | FcγRIII-γ |
| TTP | CD86 | OX40 | FcεRIβ |
| TTP | CD86 | OX40 | FcεRIγ |
| TTP | CD86 | OX40 | DAP10 |
| TTP | CD86 | OX40 | DAP12 |
| TTP | CD86 | OX40 | CD32 |
| TTP | CD86 | OX40 | CD79a |
| TTP | CD86 | OX40 | CD79b |
| TTP | CD86 | DAP10 | CD8 |
| TTP | CD86 | DAP10 | CD3ζ |
| TTP | CD86 | DAP10 | CD3δ |
| TTP | CD86 | DAP10 | CD3γ |
| TTP | CD86 | DAP10 | CD3ε |
| TTP | CD86 | DAP10 | FcγRI-γ |
| TTP | CD86 | DAP10 | FcγRIII-γ |
| TTP | CD86 | DAP10 | FcεRIβ |
| TTP | CD86 | DAP10 | FcεRIγ |
| TTP | CD86 | DAP10 | DAP10 |
| TTP | CD86 | DAP10 | DAP12 |
| TTP | CD86 | DAP10 | CD32 |
| TTP | CD86 | DAP10 | CD79a |
| TTP | CD86 | DAP10 | CD79b |
| TTP | CD86 | DAP12 | CD8 |
| TTP | CD86 | DAP12 | CD3ζ |
| TTP | CD86 | DAP12 | CD3δ |
| TTP | CD86 | DAP12 | CD3γ |
| TTP | CD86 | DAP12 | CD3ε |
| TTP | CD86 | DAP12 | FcγRI-γ |
| TTP | CD86 | DAP12 | FcγRIII-γ |
| TTP | CD86 | DAP12 | FcεRIβ |
| TTP | CD86 | DAP12 | FcεRIγ |
| TTP | CD86 | DAP12 | DAP10 |
| TTP | CD86 | DAP12 | DAP12 |
| TTP | CD86 | DAP12 | CD32 |
| TTP | CD86 | DAP12 | CD79a |
| TTP | CD86 | DAP12 | CD79b |
| TTP | CD86 | MyD88 | CD8 |
| TTP | CD86 | MyD88 | CD3ζ |
| TTP | CD86 | MyD88 | CD3δ |
| TTP | CD86 | MyD88 | CD3γ |
| TTP | CD86 | MyD88 | CD3ε |
| TTP | CD86 | MyD88 | FcγRI-γ |
| TTP | CD86 | MyD88 | FcγRIII-γ |
| TTP | CD86 | MyD88 | FcεRIβ |
| TTP | CD86 | MyD88 | FcεRIγ |
| TTP | CD86 | MyD88 | DAP10 |
| TTP | CD86 | MyD88 | DAP12 |
| TTP | CD86 | MyD88 | CD32 |
| TTP | CD86 | MyD88 | CD79a |
| TTP | CD86 | MyD88 | CD79b |
| TTP | CD86 | CD7 | CD8 |
| TTP | CD86 | CD7 | CD3ζ |
| TTP | CD86 | CD7 | CD3δ |
| TTP | CD86 | CD7 | CD3γ |
| TTP | CD86 | CD7 | CD3ε |
| TTP | CD86 | CD7 | FcγRI-γ |
| TTP | CD86 | CD7 | FcγRIII-γ |
| TTP | CD86 | CD7 | FcεRIβ |
| TTP | CD86 | CD7 | FcεRIγ |
| TTP | CD86 | CD7 | DAP10 |
| TTP | CD86 | CD7 | DAP12 |
| TTP | CD86 | CD7 | CD32 |
| TTP | CD86 | CD7 | CD79a |
| TTP | CD86 | CD7 | CD79b |
| TTP | CD86 | BTNL3 | CD8 |
| TTP | CD86 | BTNL3 | CD3ζ |
| TTP | CD86 | BTNL3 | CD3δ |
| TTP | CD86 | BTNL3 | CD3γ |
| TTP | CD86 | BTNL3 | CD3ε |
| TTP | CD86 | BTNL3 | FcγRI-γ |
| TTP | CD86 | BTNL3 | FcγRIII-γ |
| TTP | CD86 | BTNL3 | FcεRIβ |
| TTP | CD86 | BTNL3 | FcεRIγ |
| TTP | CD86 | BTNL3 | DAP10 |
| TTP | CD86 | BTNL3 | DAP12 |
| TTP | CD86 | BTNL3 | CD32 |
| TTP | CD86 | BTNL3 | CD79a |
| TTP | CD86 | BTNL3 | CD79b |
| TTP | CD86 | NKG2D | CD8 |
| TTP | CD86 | NKG2D | CD3ζ |
| TTP | CD86 | NKG2D | CD3δ |
| TTP | CD86 | NKG2D | CD3γ |
| TTP | CD86 | NKG2D | CD3ε |
| TTP | CD86 | NKG2D | FcγRI-γ |
| TTP | CD86 | NKG2D | FcγRIII-γ |
| TTP | CD86 | NKG2D | FcεRIβ |
| TTP | CD86 | NKG2D | FcεRIγ |
| TTP | CD86 | NKG2D | DAP10 |
| TTP | CD86 | NKG2D | DAP12 |
| TTP | CD86 | NKG2D | CD32 |
| TTP | CD86 | NKG2D | CD79a |
| TTP | CD86 | NKG2D | CD79b |
| TTP | OX40 | CD28 | CD8 |
| TTP | OX40 | CD28 | CD3ζ |
| TTP | OX40 | CD28 | CD3δ |
| TTP | OX40 | CD28 | CD3γ |
| TTP | OX40 | CD28 | CD3ε |
| TTP | OX40 | CD28 | FcγRI-γ |
| TTP | OX40 | CD28 | FcγRIII-γ |
| TTP | OX40 | CD28 | FcεRIβ |
| TTP | OX40 | CD28 | FcεRIγ |
| TTP | OX40 | CD28 | DAP10 |
| TTP | OX40 | CD28 | DAP12 |
| TTP | OX40 | CD28 | CD32 |
| TTP | OX40 | CD28 | CD79a |
| TTP | OX40 | CD28 | CD79b |
| TTP | OX40 | CD8 | CD8 |
| TTP | OX40 | CD8 | CD3ζ |
| TTP | OX40 | CD8 | CD3δ |
| TTP | OX40 | CD8 | CD3γ |
| TTP | OX40 | CD8 | CD3ε |
| TTP | OX40 | CD8 | FcγRI-γ |
| TTP | OX40 | CD8 | FcγRIII-γ |
| TTP | OX40 | CD8 | FcεRIβ |
| TTP | OX40 | CD8 | FcεRIγ |
| TTP | OX40 | CD8 | DAP10 |
| TTP | OX40 | CD8 | DAP12 |
| TTP | OX40 | CD8 | CD32 |
| TTP | OX40 | CD8 | CD79a |
| TTP | OX40 | CD8 | CD79b |
| TTP | OX40 | CD4 | CD8 |
| TTP | OX40 | CD4 | CD3ζ |
| TTP | OX40 | CD4 | CD3δ |
| TTP | OX40 | CD4 | CD3γ |
| TTP | OX40 | CD4 | CD3ε |
| TTP | OX40 | CD4 | FcγRI-γ |
| TTP | OX40 | CD4 | FcγRIII-γ |
| TTP | OX40 | CD4 | FcεRIβ |
| TTP | OX40 | CD4 | FcεRIγ |
| TTP | OX40 | CD4 | DAP10 |
| TTP | OX40 | CD4 | DAP12 |
| TTP | OX40 | CD4 | CD32 |
| TTP | OX40 | CD4 | CD79a |
| TTP | OX40 | CD4 | CD79b |
| TTP | OX40 | b2c | CD8 |
| TTP | OX40 | b2c | CD3ζ |
| TTP | OX40 | b2c | CD3δ |
| TTP | OX40 | b2c | CD3γ |
| TTP | OX40 | b2c | CD3ε |
| TTP | OX40 | b2c | FcγRI-γ |
| TTP | OX40 | b2c | FcγRIII-γ |
| TTP | OX40 | b2c | FcεRIβ |
| TTP | OX40 | b2c | FcεRIγ |
| TTP | OX40 | b2c | DAP10 |
| TTP | OX40 | b2c | DAP12 |
| TTP | OX40 | b2c | CD32 |
| TTP | OX40 | b2c | CD79a |
| TTP | OX40 | b2c | CD79b |
| TTP | OX40 | CD137/41BB | CD8 |
| TTP | OX40 | CD137/41BB | CD3ζ |
| TTP | OX40 | CD137/41BB | CD3δ |
| TTP | OX40 | CD137/41BB | CD3γ |
| TTP | OX40 | CD137/41BB | CD3ε |
| TTP | OX40 | CD137/41BB | FcγRI-γ |
| TTP | OX40 | CD137/41BB | FcγRIII-γ |
| TTP | OX40 | CD137/41BB | FcεRIβ |
| TTP | OX40 | CD137/41BB | FcεRIγ |
| TTP | OX40 | CD137/41BB | DAP10 |
| TTP | OX40 | CD137/41BB | DAP12 |
| TTP | OX40 | CD137/41BB | CD32 |
| TTP | OX40 | CD137/41BB | CD79a |
| TTP | OX40 | CD137/41BB | CD79b |
| TTP | OX40 | ICOS | CD8 |
| TTP | OX40 | ICOS | CD3ζ |
| TTP | OX40 | ICOS | CD3δ |
| TTP | OX40 | ICOS | CD3γ |
| TTP | OX40 | ICOS | CD3ε |
| TTP | OX40 | ICOS | FcγRI-γ |
| TTP | OX40 | ICOS | FcγRIII-γ |
| TTP | OX40 | ICOS | FcεRIβ |
| TTP | OX40 | ICOS | FcεRIγ |
| TTP | OX40 | ICOS | DAP10 |
| TTP | OX40 | ICOS | DAP12 |
| TTP | OX40 | ICOS | CD32 |
| TTP | OX40 | ICOS | CD79a |
| TTP | OX40 | ICOS | CD79b |
| TTP | OX40 | CD27 | CD8 |
| TTP | OX40 | CD27 | CD3ζ |
| TTP | OX40 | CD27 | CD3δ |
| TTP | OX40 | CD27 | CD3γ |
| TTP | OX40 | CD27 | CD3ε |
| TTP | OX40 | CD27 | FcγRI-γ |
| TTP | OX40 | CD27 | FcγRIII-γ |
| TTP | OX40 | CD27 | FcεRIβ |
| TTP | OX40 | CD27 | FcεRIγ |
| TTP | OX40 | CD27 | DAP10 |
| TTP | OX40 | CD27 | DAP12 |
| TTP | OX40 | CD27 | CD32 |
| TTP | OX40 | CD27 | CD79a |
| TTP | OX40 | CD27 | CD79b |
| TTP | OX40 | CD28δ | CD8 |
| TTP | OX40 | CD28δ | CD3ζ |
| TTP | OX40 | CD28δ | CD3δ |
| TTP | OX40 | CD28δ | CD3γ |
| TTP | OX40 | CD28δ | CD3ε |
| TTP | OX40 | CD28δ | FcγRI-γ |
| TTP | OX40 | CD28δ | FcγRIII-γ |
| TTP | OX40 | CD28δ | FcεRIβ |
| TTP | OX40 | CD28δ | FcεRIγ |
| TTP | OX40 | CD28δ | DAP10 |
| TTP | OX40 | CD28δ | DAP12 |
| TTP | OX40 | CD28δ | CD32 |
| TTP | OX40 | CD28δ | CD79a |
| TTP | OX40 | CD28δ | CD79b |
| TTP | OX40 | CD80 | CD8 |
| TTP | OX40 | CD80 | CD3ζ |
| TTP | OX40 | CD80 | CD3δ |
| TTP | OX40 | CD80 | CD3γ |
| TTP | OX40 | CD80 | CD3ε |
| TTP | OX40 | CD80 | FcγRI-γ |
| TTP | OX40 | CD80 | FcγRIII-γ |
| TTP | OX40 | CD80 | FcεRIβ |
| TTP | OX40 | CD80 | FcεRIγ |
| TTP | OX40 | CD80 | DAP10 |
| TTP | OX40 | CD80 | DAP12 |
| TTP | OX40 | CD80 | CD32 |
| TTP | OX40 | CD80 | CD79a |
| TTP | OX40 | CD80 | CD79b |
| TTP | OX40 | CD86 | CD8 |
| TTP | OX40 | CD86 | CD3ζ |
| TTP | OX40 | CD86 | CD3δ |
| TTP | OX40 | CD86 | CD3γ |
| TTP | OX40 | CD86 | CD3ε |
| TTP | OX40 | CD86 | FcγRI-γ |
| TTP | OX40 | CD86 | FcγRIII-γ |
| TTP | OX40 | CD86 | FcεRIβ |
| TTP | OX40 | CD86 | FcεRIγ |
| TTP | OX40 | CD86 | DAP10 |
| TTP | OX40 | CD86 | DAP12 |
| TTP | OX40 | CD86 | CD32 |
| TTP | OX40 | CD86 | CD79a |
| TTP | OX40 | CD86 | CD79b |
| TTP | OX40 | OX40 | CD8 |
| TTP | OX40 | OX40 | CD3ζ |
| TTP | OX40 | OX40 | CD3δ |
| TTP | OX40 | OX40 | CD3γ |
| TTP | OX40 | OX40 | CD3ε |
| TTP | OX40 | OX40 | FcγRI-γ |
| TTP | OX40 | OX40 | FcγRIII-γ |
| TTP | OX40 | OX40 | FcεRIβ |
| TTP | OX40 | OX40 | FcεRIγ |
| TTP | OX40 | OX40 | DAP10 |
| TTP | OX40 | OX40 | DAP12 |
| TTP | OX40 | OX40 | CD32 |
| TTP | OX40 | OX40 | CD79a |
| TTP | OX40 | OX40 | CD79b |
| TTP | OX40 | DAP10 | CD8 |
| TTP | OX40 | DAP10 | CD3ζ |
| TTP | OX40 | DAP10 | CD3δ |
| TTP | OX40 | DAP10 | CD3γ |
| TTP | OX40 | DAP10 | CD3ε |
| TTP | OX40 | DAP10 | FcγRI-γ |
| TTP | OX40 | DAP10 | FcγRIII-γ |
| TTP | OX40 | DAP10 | FcεRIβ |
| TTP | OX40 | DAP10 | FcεRIγ |
| TTP | OX40 | DAP10 | DAP10 |
| TTP | OX40 | DAP10 | DAP12 |
| TTP | OX40 | DAP10 | CD32 |
| TTP | OX40 | DAP10 | CD79a |
| TTP | OX40 | DAP10 | CD79b |
| TTP | OX40 | DAP12 | CD8 |
| TTP | OX40 | DAP12 | CD3ζ |
| TTP | OX40 | DAP12 | CD3δ |
| TTP | OX40 | DAP12 | CD3γ |
| TTP | OX40 | DAP12 | CD3ε |
| TTP | OX40 | DAP12 | FcγRI-γ |
| TTP | OX40 | DAP12 | FcγRIII-γ |
| TTP | OX40 | DAP12 | FcεRIβ |
| TTP | OX40 | DAP12 | FcεRIγ |
| TTP | OX40 | DAP12 | DAP10 |
| TTP | OX40 | DAP12 | DAP12 |
| TTP | OX40 | DAP12 | CD32 |
| TTP | OX40 | DAP12 | CD79a |
| TTP | OX40 | DAP12 | CD79b |
| TTP | OX40 | MyD88 | CD8 |
| TTP | OX40 | MyD88 | CD3ζ |
| TTP | OX40 | MyD88 | CD3δ |
| TTP | OX40 | MyD88 | CD3γ |
| TTP | OX40 | MyD88 | CD3ε |
| TTP | OX40 | MyD88 | FcγRI-γ |
| TTP | OX40 | MyD88 | FcγRIII-γ |
| TTP | OX40 | MyD88 | FcεRIβ |
| TTP | OX40 | MyD88 | FcεRIγ |
| TTP | OX40 | MyD88 | DAP10 |
| TTP | OX40 | MyD88 | DAP12 |
| TTP | OX40 | MyD88 | CD32 |
| TTP | OX40 | MyD88 | CD79a |
| TTP | OX40 | MyD88 | CD79b |
| TTP | OX40 | CD7 | CD8 |
| TTP | OX40 | CD7 | CD3ζ |
| TTP | OX40 | CD7 | CD3δ |
| TTP | OX40 | CD7 | CD3γ |
| TTP | OX40 | CD7 | CD3ε |
| TTP | OX40 | CD7 | FcγRI-γ |
| TTP | OX40 | CD7 | FcγRIII-γ |
| TTP | OX40 | CD7 | FcεRIβ |
| TTP | OX40 | CD7 | FcεRIγ |
| TTP | OX40 | CD7 | DAP10 |
| TTP | OX40 | CD7 | DAP12 |
| TTP | OX40 | CD7 | CD32 |
| TTP | OX40 | CD7 | CD79a |
| TTP | OX40 | CD7 | CD79b |
| TTP | OX40 | BTNL3 | CD8 |
| TTP | OX40 | BTNL3 | CD3ζ |
| TTP | OX40 | BTNL3 | CD3δ |
| TTP | OX40 | BTNL3 | CD3γ |
| TTP | OX40 | BTNL3 | CD3ε |
| TTP | OX40 | BTNL3 | FcγRI-γ |
| TTP | OX40 | BTNL3 | FcγRIII-γ |
| TTP | OX40 | BTNL3 | FcεRIβ |
| TTP | OX40 | BTNL3 | FcεRIγ |
| TTP | OX40 | BTNL3 | DAP10 |
| TTP | OX40 | BTNL3 | DAP12 |
| TTP | OX40 | BTNL3 | CD32 |
| TTP | OX40 | BTNL3 | CD79a |
| TTP | OX40 | BTNL3 | CD79b |
| TTP | OX40 | NKG2D | CD8 |
| TTP | OX40 | NKG2D | CD3ζ |
| TTP | OX40 | NKG2D | CD3δ |
| TTP | OX40 | NKG2D | CD3γ |
| TTP | OX40 | NKG2D | CD3ε |
| TTP | OX40 | NKG2D | FcγRI-γ |
| TTP | OX40 | NKG2D | FcγRIII-γ |
| TTP | OX40 | NKG2D | FcεRIβ |
| TTP | OX40 | NKG2D | FcεRIγ |
| TTP | OX40 | NKG2D | DAP10 |
| TTP | OX40 | NKG2D | DAP12 |
| TTP | OX40 | NKG2D | CD32 |
| TTP | OX40 | NKG2D | CD79a |
| TTP | OX40 | NKG2D | CD79b |
| TTP | DAP10 | CD28 | CD8 |
| TTP | DAP10 | CD28 | CD3ζ |
| TTP | DAP10 | CD28 | CD3δ |
| TTP | DAP10 | CD28 | CD3γ |
| TTP | DAP10 | CD28 | CD3ε |
| TTP | DAP10 | CD28 | FcγRI-γ |
| TTP | DAP10 | CD28 | FcγRIII-γ |
| TTP | DAP10 | CD28 | FcεRIβ |
| TTP | DAP10 | CD28 | FcεRIγ |
| TTP | DAP10 | CD28 | DAP10 |
| TTP | DAP10 | CD28 | DAP12 |
| TTP | DAP10 | CD28 | CD32 |
| TTP | DAP10 | CD28 | CD79a |
| TTP | DAP10 | CD28 | CD79b |
| TTP | DAP10 | CD8 | CD8 |
| TTP | DAP10 | CD8 | CD3ζ |
| TTP | DAP10 | CD8 | CD3δ |
| TTP | DAP10 | CD8 | CD3γ |
| TTP | DAP10 | CD8 | CD3ε |
| TTP | DAP10 | CD8 | FcγRI-γ |
| TTP | DAP10 | CD8 | FcγRIII-γ |
| TTP | DAP10 | CD8 | FcεRIβ |
| TTP | DAP10 | CD8 | FcεRIγ |
| TTP | DAP10 | CD8 | DAP10 |
| TTP | DAP10 | CD8 | DAP12 |
| TTP | DAP10 | CD8 | CD32 |
| TTP | DAP10 | CD8 | CD79a |
| TTP | DAP10 | CD8 | CD79b |
| TTP | DAP10 | CD4 | CD8 |
| TTP | DAP10 | CD4 | CD3ζ |
| TTP | DAP10 | CD4 | CD3δ |
| TTP | DAP10 | CD4 | CD3γ |
| TTP | DAP10 | CD4 | CD3ε |
| TTP | DAP10 | CD4 | FcγRI-γ |
| TTP | DAP10 | CD4 | FcγRIII-γ |
| TTP | DAP10 | CD4 | FcεRIβ |
| TTP | DAP10 | CD4 | FcεRIγ |
| TTP | DAP10 | CD4 | DAP10 |
| TTP | DAP10 | CD4 | DAP12 |
| TTP | DAP10 | CD4 | CD32 |
| TTP | DAP10 | CD4 | CD79a |
| TTP | DAP10 | CD4 | CD79b |
| TTP | DAP10 | b2c | CD8 |
| TTP | DAP10 | b2c | CD3ζ |
| TTP | DAP10 | b2c | CD3δ |
| TTP | DAP10 | b2c | CD3γ |
| TTP | DAP10 | b2c | CD3ε |
| TTP | DAP10 | b2c | FcγRI-γ |
| TTP | DAP10 | b2c | FcγRIII-γ |
| TTP | DAP10 | b2c | FcεRIβ |
| TTP | DAP10 | b2c | FcεRIγ |
| TTP | DAP10 | b2c | DAP10 |
| TTP | DAP10 | b2c | DAP12 |
| TTP | DAP10 | b2c | CD32 |
| TTP | DAP10 | b2c | CD79a |
| TTP | DAP10 | b2c | CD79b |
| TTP | DAP10 | CD137/41BB | CD8 |
| TTP | DAP10 | CD137/41BB | CD3ζ |
| TTP | DAP10 | CD137/41BB | CD3δ |
| TTP | DAP10 | CD137/41BB | CD3γ |
| TTP | DAP10 | CD137/41BB | CD3ε |
| TTP | DAP10 | CD137/41BB | FcγRI-γ |
| TTP | DAP10 | CD137/41BB | FcγRIII-γ |
| TTP | DAP10 | CD137/41BB | FcεRIβ |
| TTP | DAP10 | CD137/41BB | FcεRIγ |
| TTP | DAP10 | CD137/41BB | DAP10 |
| TTP | DAP10 | CD137/41BB | DAP12 |
| TTP | DAP10 | CD137/41BB | CD32 |
| TTP | DAP10 | CD137/41BB | CD79a |
| TTP | DAP10 | CD137/41BB | CD79b |
| TTP | DAP10 | ICOS | CD8 |
| TTP | DAP10 | ICOS | CD3ζ |
| TTP | DAP10 | ICOS | CD3δ |
| TTP | DAP10 | ICOS | CD3γ |
| TTP | DAP10 | ICOS | CD3ε |
| TTP | DAP10 | ICOS | FcγRI-γ |
| TTP | DAP10 | ICOS | FcγRIII-γ |
| TTP | DAP10 | ICOS | FcεRIβ |
| TTP | DAP10 | ICOS | FcεRIγ |
| TTP | DAP10 | ICOS | DAP10 |
| TTP | DAP10 | ICOS | DAP12 |
| TTP | DAP10 | ICOS | CD32 |
| TTP | DAP10 | ICOS | CD79a |
| TTP | DAP10 | ICOS | CD79b |
| TTP | DAP10 | CD27 | CD8 |
| TTP | DAP10 | CD27 | CD3ζ |
| TTP | DAP10 | CD27 | CD3δ |
| TTP | DAP10 | CD27 | CD3γ |
| TTP | DAP10 | CD27 | CD3ε |
| TTP | DAP10 | CD27 | FcγRI-γ |
| TTP | DAP10 | CD27 | FcγRIII-γ |
| TTP | DAP10 | CD27 | FcεRIβ |
| TTP | DAP10 | CD27 | FcεRIγ |
| TTP | DAP10 | CD27 | DAP10 |
| TTP | DAP10 | CD27 | DAP12 |
| TTP | DAP10 | CD27 | CD32 |
| TTP | DAP10 | CD27 | CD79a |
| TTP | DAP10 | CD27 | CD79b |
| TTP | DAP10 | CD28δ | CD8 |
| TTP | DAP10 | CD28δ | CD3ζ |
| TTP | DAP10 | CD28δ | CD3δ |
| TTP | DAP10 | CD28δ | CD3γ |
| TTP | DAP10 | CD28δ | CD3ε |
| TTP | DAP10 | CD28δ | FcγRI-γ |
| TTP | DAP10 | CD28δ | FcγRIII-γ |
| TTP | DAP10 | CD28δ | FcεRIβ |
| TTP | DAP10 | CD28δ | FcεRIγ |
| TTP | DAP10 | CD28δ | DAP10 |
| TTP | DAP10 | CD28δ | DAP12 |
| TTP | DAP10 | CD28δ | CD32 |
| TTP | DAP10 | CD28δ | CD79a |
| TTP | DAP10 | CD28δ | CD79b |
| TTP | DAP10 | CD80 | CD8 |
| TTP | DAP10 | CD80 | CD3ζ |
| TTP | DAP10 | CD80 | CD3δ |
| TTP | DAP10 | CD80 | CD3γ |
| TTP | DAP10 | CD80 | CD3ε |
| TTP | DAP10 | CD80 | FcγRI-γ |
| TTP | DAP10 | CD80 | FcγRIII-γ |
| TTP | DAP10 | CD80 | FcεRIβ |
| TTP | DAP10 | CD80 | FcεRIγ |
| TTP | DAP10 | CD80 | DAP10 |
| TTP | DAP10 | CD80 | DAP12 |
| TTP | DAP10 | CD80 | CD32 |
| TTP | DAP10 | CD80 | CD79a |
| TTP | DAP10 | CD80 | CD79b |
| TTP | DAP10 | CD86 | CD8 |
| TTP | DAP10 | CD86 | CD3ζ |
| TTP | DAP10 | CD86 | CD3δ |
| TTP | DAP10 | CD86 | CD3γ |
| TTP | DAP10 | CD86 | CD3ε |
| TTP | DAP10 | CD86 | FcγRI-γ |
| TTP | DAP10 | CD86 | FcγRIII-γ |
| TTP | DAP10 | CD86 | FcγRIβ |
| TTP | DAP10 | CD86 | FcγRIγ |
| TTP | DAP10 | CD86 | DAP10 |
| TTP | DAP10 | CD86 | DAP12 |
| TTP | DAP10 | CD86 | CD32 |
| TTP | DAP10 | CD86 | CD79a |
| TTP | DAP10 | CD86 | CD79b |
| TTP | DAP10 | OX40 | CD8 |
| TTP | DAP10 | OX40 | CD3ζ |
| TTP | DAP10 | OX40 | CD3δ |
| TTP | DAP10 | OX40 | CD3γ |
| TTP | DAP10 | OX40 | CD3ε |
| TTP | DAP10 | OX40 | FcγRI-γ |
| TTP | DAP10 | OX40 | FcγRIII-γ |
| TTP | DAP10 | OX40 | FcεRIβ |
| TTP | DAP10 | OX40 | FcεRIγ |
| TTP | DAP10 | OX40 | DAP10 |
| TTP | DAP10 | OX40 | DAP12 |
| TTP | DAP10 | OX40 | CD32 |
| TTP | DAP10 | OX40 | CD79a |
| TTP | DAP10 | OX40 | CD79b |
| TTP | DAP10 | DAP10 | CD8 |
| TTP | DAP10 | DAP10 | CD3ζ |
| TTP | DAP10 | DAP10 | CD3δ |
| TTP | DAP10 | DAP10 | CD3γ |
| TTP | DAP10 | DAP10 | CD3ε |
| TTP | DAP10 | DAP10 | FcγRI-γ |
| TTP | DAP10 | DAP10 | FcγRIII-γ |
| TTP | DAP10 | DAP10 | FcεRIβ |
| TTP | DAP10 | DAP10 | FcεRIγ |
| TTP | DAP10 | DAP10 | DAP10 |
| TTP | DAP10 | DAP10 | DAP12 |
| TTP | DAP10 | DAP10 | CD32 |
| TTP | DAP10 | DAP10 | CD79a |
| TTP | DAP10 | DAP10 | CD79b |
| TTP | DAP10 | DAP12 | CD8 |
| TTP | DAP10 | DAP12 | CD3ζ |
| TTP | DAP10 | DAP12 | CD3δ |
| TTP | DAP10 | DAP12 | CD3γ |
| TTP | DAP10 | DAP12 | CD3ε |
| TTP | DAP10 | DAP12 | FcγRI-γ |
| TTP | DAP10 | DAP12 | FcγRIII-γ |
| TTP | DAP10 | DAP12 | FcεRIβ |
| TTP | DAP10 | DAP12 | FcεRIγ |
| TTP | DAP10 | DAP12 | DAP10 |
| TTP | DAP10 | DAP12 | DAP12 |
| TTP | DAP10 | DAP12 | CD32 |
| TTP | DAP10 | DAP12 | CD79a |
| TTP | DAP10 | DAP12 | CD79b |
| TTP | DAP10 | MyD88 | CD8 |
| TTP | DAP10 | MyD88 | CD3ζ |
| TTP | DAP10 | MyD88 | CD3δ |
| TTP | DAP10 | MyD88 | CD3γ |
| TTP | DAP10 | MyD88 | CD3ε |
| TTP | DAP10 | MyD88 | FcγRI-γ |
| TTP | DAP10 | MyD88 | FcγRIII-γ |
| TTP | DAP10 | MyD88 | FcεRIβ |
| TTP | DAP10 | MyD88 | FcεRIγ |
| TTP | DAP10 | MyD88 | DAP10 |
| TTP | DAP10 | MyD88 | DAP12 |
| TTP | DAP10 | MyD88 | CD32 |
| TTP | DAP10 | MyD88 | CD79a |
| TTP | DAP10 | MyD88 | CD79b |
| TTP | DAP10 | CD7 | CD8 |
| TTP | DAP10 | CD7 | CD3ζ |
| TTP | DAP10 | CD7 | CD3δ |
| TTP | DAP10 | CD7 | CD3γ |
| TTP | DAP10 | CD7 | CD3ε |
| TTP | DAP10 | CD7 | FcγRI-γ |
| TTP | DAP10 | CD7 | FcγRIII-γ |
| TTP | DAP10 | CD7 | FcεRIβ |
| TTP | DAP10 | CD7 | FcεRIγ |
| TTP | DAP10 | CD7 | DAP10 |
| TTP | DAP10 | CD7 | DAP12 |
| TTP | DAP10 | CD7 | CD32 |
| TTP | DAP10 | CD7 | CD79a |
| TTP | DAP10 | CD7 | CD79b |
| TTP | DAP10 | BTNL3 | CD8 |
| TTP | DAP10 | BTNL3 | CD3ζ |
| TTP | DAP10 | BTNL3 | CD3δ |
| TTP | DAP10 | BTNL3 | CD3γ |
| TTP | DAP10 | BTNL3 | CD3ε |
| TTP | DAP10 | BTNL3 | FcγRI-γ |
| TTP | DAP10 | BTNL3 | FcγRIII-γ |
| TTP | DAP10 | BTNL3 | FcεRIβ |
| TTP | DAP10 | BTNL3 | FcεRIγ |
| TTP | DAP10 | BTNL3 | DAP10 |
| TTP | DAP10 | BTNL3 | DAP12 |
| TTP | DAP10 | BTNL3 | CD32 |
| TTP | DAP10 | BTNL3 | CD79a |
| TTP | DAP10 | BTNL3 | CD79b |
| TTP | DAP10 | NKG2D | CD8 |
| TTP | DAP10 | NKG2D | CD3ζ |
| TTP | DAP10 | NKG2D | CD3δ |
| TTP | DAP10 | NKG2D | CD3γ |
| TTP | DAP10 | NKG2D | CD3ε |
| TTP | DAP10 | NKG2D | FcγRI-γ |
| TTP | DAP10 | NKG2D | FcγRIII-γ |
| TTP | DAP10 | NKG2D | FcεRIβ |
| TTP | DAP10 | NKG2D | FcεRIγ |
| TTP | DAP10 | NKG2D | DAP10 |
| TTP | DAP10 | NKG2D | DAP12 |
| TTP | DAP10 | NKG2D | CD32 |
| TTP | DAP10 | NKG2D | CD79a |
| TTP | DAP10 | NKG2D | CD79b |
| TTP | DAP12 | CD28 | CD8 |
| TTP | DAP12 | CD28 | CD3ζ |
| TTP | DAP12 | CD28 | CD3δ |
| TTP | DAP12 | CD28 | CD3γ |
| TTP | DAP12 | CD28 | CD3ε |
| TTP | DAP12 | CD28 | FcγRI-γ |
| TTP | DAP12 | CD28 | FcγRIII-γ |
| TTP | DAP12 | CD28 | FcεRIβ |
| TTP | DAP12 | CD28 | FcεRIγ |
| TTP | DAP12 | CD28 | DAP10 |
| TTP | DAP12 | CD28 | DAP12 |
| TTP | DAP12 | CD28 | CD32 |
| TTP | DAP12 | CD28 | CD79a |
| TTP | DAP12 | CD28 | CD79b |
| TTP | DAP12 | CD8 | CD8 |
| TTP | DAP12 | CD8 | CD3ζ |
| TTP | DAP12 | CD8 | CD3δ |
| TTP | DAP12 | CD8 | CD3γ |
| TTP | DAP12 | CD8 | CD3ε |
| TTP | DAP12 | CD8 | FcγRI-γ |
| TTP | DAP12 | CD8 | FcγRIII-γ |
| TTP | DAP12 | CD8 | FcεRIβ |
| TTP | DAP12 | CD8 | FcεRIγ |
| TTP | DAP12 | CD8 | DAP10 |
| TTP | DAP12 | CD8 | DAP12 |
| TTP | DAP12 | CD8 | CD32 |
| TTP | DAP12 | CD8 | CD79a |
| TTP | DAP12 | CD8 | CD79b |
| TTP | DAP12 | CD4 | CD8 |
| TTP | DAP12 | CD4 | CD3ζ |
| TTP | DAP12 | CD4 | CD3δ |
| TTP | DAP12 | CD4 | CD3γ |
| TTP | DAP12 | CD4 | CD3ε |
| TTP | DAP12 | CD4 | FcγRI-γ |
| TTP | DAP12 | CD4 | FcγRIII-γ |
| TTP | DAP12 | CD4 | FcεRIβ |
| TTP | DAP12 | CD4 | FcεRIγ |
| TTP | DAP12 | CD4 | DAP10 |
| TTP | DAP12 | CD4 | DAP12 |
| TTP | DAP12 | CD4 | CD32 |
| TTP | DAP12 | CD4 | CD79a |
| TTP | DAP12 | CD4 | CD79b |
| TTP | DAP12 | b2c | CD8 |
| TTP | DAP12 | b2c | CD3ζ |
| TTP | DAP12 | b2c | CD3δ |
| TTP | DAP12 | b2c | CD3γ |
| TTP | DAP12 | b2c | CD3ε |
| TTP | DAP12 | b2c | FcγRI-γ |
| TTP | DAP12 | b2c | FcγRIII-γ |
| TTP | DAP12 | b2c | FcεRIβ |
| TTP | DAP12 | b2c | FcεRIγ |
| TTP | DAP12 | b2c | DAP10 |
| TTP | DAP12 | b2c | DAP12 |
| TTP | DAP12 | b2c | CD32 |
| TTP | DAP12 | b2c | CD79a |
| TTP | DAP12 | b2c | CD79b |
| TTP | DAP12 | CD137/41BB | CD8 |
| TTP | DAP12 | CD137/41BB | CD3ζ |
| TTP | DAP12 | CD137/41BB | CD3δ |
| TTP | DAP12 | CD137/41BB | CD3γ |
| TTP | DAP12 | CD137/41BB | CD3ε |
| TTP | DAP12 | CD137/41BB | FcγRI-γ |
| TTP | DAP12 | CD137/41BB | FcγRIII-γ |
| TTP | DAP12 | CD137/41BB | FcεRIβ |
| TTP | DAP12 | CD137/41BB | FcεRIγ |
| TTP | DAP12 | CD137/41BB | DAP10 |
| TTP | DAP12 | CD137/41BB | DAP12 |
| TTP | DAP12 | CD137/41BB | CD32 |
| TTP | DAP12 | CD137/41BB | CD79a |
| TTP | DAP12 | CD137/41BB | CD79b |
| TTP | DAP12 | ICOS | CD8 |
| TTP | DAP12 | ICOS | CD3ζ |
| TTP | DAP12 | ICOS | CD3δ |
| TTP | DAP12 | ICOS | CD3γ |
| TTP | DAP12 | ICOS | CD3ε |
| TTP | DAP12 | ICOS | FcγRI-γ |
| TTP | DAP12 | ICOS | FcγRIII-γ |
| TTP | DAP12 | ICOS | FcεRIβ |
| TTP | DAP12 | ICOS | FcεRIγ |
| TTP | DAP12 | ICOS | DAP10 |
| TTP | DAP12 | ICOS | DAP12 |
| TTP | DAP12 | ICOS | CD32 |
| TTP | DAP12 | ICOS | CD79a |
| TTP | DAP12 | ICOS | CD79b |
| TTP | DAP12 | CD27 | CD8 |
| TTP | DAP12 | CD27 | CD3ζ |
| TTP | DAP12 | CD27 | CD3δ |
| TTP | DAP12 | CD27 | CD3γ |
| TTP | DAP12 | CD27 | CD3ε |
| TTP | DAP12 | CD27 | FcγRI-γ |
| TTP | DAP12 | CD27 | FcγRIII-γ |
| TTP | DAP12 | CD27 | FcεRIβ |
| TTP | DAP12 | CD27 | FcεRIγ |
| TTP | DAP12 | CD27 | DAP10 |
| TTP | DAP12 | CD27 | DAP12 |
| TTP | DAP12 | CD27 | CD32 |
| TTP | DAP12 | CD27 | CD79a |
| TTP | DAP12 | CD27 | CD79b |
| TTP | DAP12 | CD28δ | CD8 |
| TTP | DAP12 | CD28δ | CD3ζ |
| TTP | DAP12 | CD28δ | CD3δ |
| TTP | DAP12 | CD28δ | CD3γ |
| TTP | DAP12 | CD28δ | CD3ε |
| TTP | DAP12 | CD28δ | FcγRI-γ |
| TTP | DAP12 | CD28δ | FcγRIII-γ |
| TTP | DAP12 | CD28δ | FcεRIβ |
| TTP | DAP12 | CD28δ | FcεRIγ |
| TTP | DAP12 | CD28δ | DAP10 |
| TTP | DAP12 | CD28δ | DAP12 |
| TTP | DAP12 | CD28δ | CD32 |
| TTP | DAP12 | CD28δ | CD79a |
| TTP | DAP12 | CD28δ | CD79b |
| TTP | DAP12 | CD80 | CD8 |
| TTP | DAP12 | CD80 | CD3ζ |
| TTP | DAP12 | CD80 | CD3δ |
| TTP | DAP12 | CD80 | CD3γ |
| TTP | DAP12 | CD80 | CD3ε |
| TTP | DAP12 | CD80 | FcγRI-γ |
| TTP | DAP12 | CD80 | FcγRIII-γ |
| TTP | DAP12 | CD80 | FcεRIβ |
| TTP | DAP12 | CD80 | FcεRIγ |
| TTP | DAP12 | CD80 | DAP10 |
| TTP | DAP12 | CD80 | DAP12 |
| TTP | DAP12 | CD80 | CD32 |
| TTP | DAP12 | CD80 | CD79a |
| TTP | DAP12 | CD80 | CD79b |
| TTP | DAP12 | CD86 | CD8 |
| TTP | DAP12 | CD86 | CD3ζ |
| TTP | DAP12 | CD86 | CD3δ |
| TTP | DAP12 | CD86 | CD3γ |
| TTP | DAP12 | CD86 | CD3ε |
| TTP | DAP12 | CD86 | FcγRI-γ |
| TTP | DAP12 | CD86 | FcγRIII-γ |
| TTP | DAP12 | CD86 | FcεRIβ |
| TTP | DAP12 | CD86 | FcεRIγ |
| TTP | DAP12 | CD86 | DAP10 |
| TTP | DAP12 | CD86 | DAP12 |
| TTP | DAP12 | CD86 | CD32 |
| TTP | DAP12 | CD86 | CD79a |
| TTP | DAP12 | CD86 | CD79b |
| TTP | DAP12 | OX40 | CD8 |
| TTP | DAP12 | OX40 | CD3ζ |
| TTP | DAP12 | OX40 | CD3δ |
| TTP | DAP12 | OX40 | CD3γ |
| TTP | DAP12 | OX40 | CD3ε |
| TTP | DAP12 | OX40 | FcγRI-γ |
| TTP | DAP12 | OX40 | FcγRIII-γ |
| TTP | DAP12 | OX40 | FcεRIβ |
| TTP | DAP12 | OX40 | FcεRIγ |
| TTP | DAP12 | OX40 | DAP10 |
| TTP | DAP12 | OX40 | DAP12 |
| TTP | DAP12 | OX40 | CD32 |
| TTP | DAP12 | OX40 | CD79a |
| TTP | DAP12 | OX40 | CD79b |
| TTP | DAP12 | DAP10 | CD8 |
| TTP | DAP12 | DAP10 | CD3ζ |
| TTP | DAP12 | DAP10 | CD3δ |
| TTP | DAP12 | DAP10 | CD3γ |
| TTP | DAP12 | DAP10 | CD3ε |
| TTP | DAP12 | DAP10 | FcγRI-γ |
| TTP | DAP12 | DAP10 | FcγRIII-γ |
| TTP | DAP12 | DAP10 | FcεRIβ |
| TTP | DAP12 | DAP10 | FcεRIγ |
| TTP | DAP12 | DAP10 | DAP10 |
| TTP | DAP12 | DAP10 | DAP12 |
| TTP | DAP12 | DAP10 | CD32 |
| TTP | DAP12 | DAP10 | CD79a |
| TTP | DAP12 | DAP10 | CD79b |
| TTP | DAP12 | DAP12 | CD8 |
| TTP | DAP12 | DAP12 | CD3ζ |
| TTP | DAP12 | DAP12 | CD3δ |
| TTP | DAP12 | DAP12 | CD3γ |
| TTP | DAP12 | DAP12 | CD3ε |
| TTP | DAP12 | DAP12 | FcγRI-γ |
| TTP | DAP12 | DAP12 | FcγRIII-γ |
| TTP | DAP12 | DAP12 | FcεRIβ |
| TTP | DAP12 | DAP12 | FcεRIγ |
| TTP | DAP12 | DAP12 | DAP10 |
| TTP | DAP12 | DAP12 | DAP12 |
| TTP | DAP12 | DAP12 | CD32 |
| TTP | DAP12 | DAP12 | CD79a |
| TTP | DAP12 | DAP12 | CD79b |
| TTP | DAP12 | MyD88 | CD8 |
| TTP | DAP12 | MyD88 | CD3ζ |
| TTP | DAP12 | MyD88 | CD3δ |
| TTP | DAP12 | MyD88 | CD3γ |
| TTP | DAP12 | MyD88 | CD3ε |
| TTP | DAP12 | MyD88 | FcγRI-γ |
| TTP | DAP12 | MyD88 | FcγRIII-γ |
| TTP | DAP12 | MyD88 | FcεRIβ |
| TTP | DAP12 | MyD88 | FcεRIγ |
| TTP | DAP12 | MyD88 | DAP10 |
| TTP | DAP12 | MyD88 | DAP12 |
| TTP | DAP12 | MyD88 | CD32 |
| TTP | DAP12 | MyD88 | CD79a |
| TTP | DAP12 | MyD88 | CD79b |
| TTP | DAP12 | CD7 | CD8 |
| TTP | DAP12 | CD7 | CD3ζ |
| TTP | DAP12 | CD7 | CD3δ |
| TTP | DAP12 | CD7 | CD3γ |
| TTP | DAP12 | CD7 | CD3ε |
| TTP | DAP12 | CD7 | FcγRI-γ |
| TTP | DAP12 | CD7 | FcγRIII-γ |
| TTP | DAP12 | CD7 | FcεRIβ |
| TTP | DAP12 | CD7 | FcεRIγ |
| TTP | DAP12 | CD7 | DAP10 |
| TTP | DAP12 | CD7 | DAP12 |
| TTP | DAP12 | CD7 | CD32 |
| TTP | DAP12 | CD7 | CD79a |
| TTP | DAP12 | CD7 | CD79b |
| TTP | DAP12 | BTNL3 | CD8 |
| TTP | DAP12 | BTNL3 | CD3ζ |
| TTP | DAP12 | BTNL3 | CD3δ |
| TTP | DAP12 | BTNL3 | CD3γ |
| TTP | DAP12 | BTNL3 | CD3ε |
| TTP | DAP12 | BTNL3 | FcγRI-γ |
| TTP | DAP12 | BTNL3 | FcγRIII-γ |
| TTP | DAP12 | BTNL3 | FcεRIβ |
| TTP | DAP12 | BTNL3 | FcεRIγ |
| TTP | DAP12 | BTNL3 | DAP10 |
| TTP | DAP12 | BTNL3 | DAP12 |
| TTP | DAP12 | BTNL3 | CD32 |
| TTP | DAP12 | BTNL3 | CD79a |
| TTP | DAP12 | BTNL3 | CD79b |
| TTP | DAP12 | NKG2D | CD8 |
| TTP | DAP12 | NKG2D | CD3ζ |
| TTP | DAP12 | NKG2D | CD3δ |
| TTP | DAP12 | NKG2D | CD3γ |
| TTP | DAP12 | NKG2D | CD3ε |
| TTP | DAP12 | NKG2D | FcγRI-γ |
| TTP | DAP12 | NKG2D | FcγRIII-γ |
| TTP | DAP12 | NKG2D | FcεRIβ |
| TTP | DAP12 | NKG2D | FcεRIγ |
| TTP | DAP12 | NKG2D | DAP10 |
| TTP | DAP12 | NKG2D | DAP12 |
| TTP | DAP12 | NKG2D | CD32 |
| TTP | DAP12 | NKG2D | CD79a |
| TTP | DAP12 | NKG2D | CD79b |
| TTP | MyD88 | CD28 | CD8 |
| TTP | MyD88 | CD28 | CD3ζ |
| TTP | MyD88 | CD28 | CD3δ |
| TTP | MyD88 | CD28 | CD3γ |
| TTP | MyD88 | CD28 | CD3ε |
| TTP | MyD88 | CD28 | FcγRI-γ |
| TTP | MyD88 | CD28 | FcγRIII-γ |
| TTP | MyD88 | CD28 | FcεRIβ |
| TTP | MyD88 | CD28 | FcεRIγ |
| TTP | MyD88 | CD28 | DAP10 |
| TTP | MyD88 | CD28 | DAP12 |
| TTP | MyD88 | CD28 | CD32 |
| TTP | MyD88 | CD28 | CD79a |
| TTP | MyD88 | CD28 | CD79b |
| TTP | MyD88 | CD8 | CD8 |
| TTP | MyD88 | CD8 | CD3ζ |
| TTP | MyD88 | CD8 | CD3δ |
| TTP | MyD88 | CD8 | CD3γ |
| TTP | MyD88 | CD8 | CD3ε |
| TTP | MyD88 | CD8 | FcγRI-γ |
| TTP | MyD88 | CD8 | FcγRIII-γ |
| TTP | MyD88 | CD8 | FcεRIβ |
| TTP | MyD88 | CD8 | FcεRIγ |
| TTP | MyD88 | CD8 | DAP10 |
| TTP | MyD88 | CD8 | DAP12 |
| TTP | MyD88 | CD8 | CD32 |
| TTP | MyD88 | CD8 | CD79a |
| TTP | MyD88 | CD8 | CD79b |
| TTP | MyD88 | CD4 | CD8 |
| TTP | MyD88 | CD4 | CD3ζ |
| TTP | MyD88 | CD4 | CD3δ |
| TTP | MyD88 | CD4 | CD3γ |
| TTP | MyD88 | CD4 | CD3ε |
| TTP | MyD88 | CD4 | FcγRI-γ |
| TTP | MyD88 | CD4 | FcγRIII-γ |
| TTP | MyD88 | CD4 | FcεRIβ |
| TTP | MyD88 | CD4 | FcεRIγ |
| TTP | MyD88 | CD4 | DAP10 |
| TTP | MyD88 | CD4 | DAP12 |
| TTP | MyD88 | CD4 | CD32 |
| TTP | MyD88 | CD4 | CD79a |
| TTP | MyD88 | CD4 | CD79b |
| TTP | MyD88 | b2c | CD8 |
| TTP | MyD88 | b2c | CD3ζ |
| TTP | MyD88 | b2c | CD3δ |
| TTP | MyD88 | b2c | CD3γ |
| TTP | MyD88 | b2c | CD3ε |
| TTP | MyD88 | b2c | FcγRI-γ |
| TTP | MyD88 | b2c | FcγRIII-γ |
| TTP | MyD88 | b2c | FcεRIβ |
| TTP | MyD88 | b2c | FcεRIγ |
| TTP | MyD88 | b2c | DAP10 |
| TTP | MyD88 | b2c | DAP12 |
| TTP | MyD88 | b2c | CD32 |
| TTP | MyD88 | b2c | CD79a |
| TTP | MyD88 | b2c | CD79b |
| TTP | MyD88 | CD137/41BB | CD8 |
| TTP | MyD88 | CD137/41BB | CD3ζ |
| TTP | MyD88 | CD137/41BB | CD3δ |
| TTP | MyD88 | CD137/41BB | CD3γ |
| TTP | MyD88 | CD137/41BB | CD3ε |
| TTP | MyD88 | CD137/41BB | FcγRI-γ |
| TTP | MyD88 | CD137/41BB | FcγRIII-γ |
| TTP | MyD88 | CD137/41BB | FcεRIβ |
| TTP | MyD88 | CD137/41BB | FcεRIγ |
| TTP | MyD88 | CD137/41BB | DAP10 |
| TTP | MyD88 | CD137/41BB | DAP12 |
| TTP | MyD88 | CD137/41BB | CD32 |
| TTP | MyD88 | CD137/41BB | CD79a |
| TTP | MyD88 | CD137/41BB | CD79b |
| TTP | MyD88 | ICOS | CD8 |
| TTP | MyD88 | ICOS | CD3ζ |
| TTP | MyD88 | ICOS | CD3δ |
| TTP | MyD88 | ICOS | CD3γ |
| TTP | MyD88 | ICOS | CD3ε |
| TTP | MyD88 | ICOS | FcγRI-γ |
| TTP | MyD88 | ICOS | FcγRIII-γ |
| TTP | MyD88 | ICOS | FcεRIβ |
| TTP | MyD88 | ICOS | FcεRIγ |
| TTP | MyD88 | ICOS | DAP10 |
| TTP | MyD88 | ICOS | DAP12 |
| TTP | MyD88 | ICOS | CD32 |
| TTP | MyD88 | ICOS | CD79a |
| TTP | MyD88 | ICOS | CD79b |
| TTP | MyD88 | CD27 | CD8 |
| TTP | MyD88 | CD27 | CD3ζ |
| TTP | MyD88 | CD27 | CD3δ |
| TTP | MyD88 | CD27 | CD3γ |
| TTP | MyD88 | CD27 | CD3ε |
| TTP | MyD88 | CD27 | FcγRI-γ |
| TTP | MyD88 | CD27 | FcγRIII-γ |
| TTP | MyD88 | CD27 | FcεRIβ |
| TTP | MyD88 | CD27 | FcεRIγ |
| TTP | MyD88 | CD27 | DAP10 |
| TTP | MyD88 | CD27 | DAP12 |
| TTP | MyD88 | CD27 | CD32 |
| TTP | MyD88 | CD27 | CD79a |
| TTP | MyD88 | CD27 | CD79b |
| TTP | MyD88 | CD28δ | CD8 |
| TTP | MyD88 | CD28δ | CD3ζ |
| TTP | MyD88 | CD28δ | CD3δ |
| TTP | MyD88 | CD28δ | CD3γ |
| TTP | MyD88 | CD28δ | CD3ε |
| TTP | MyD88 | CD28δ | FcγRI-γ |
| TTP | MyD88 | CD28δ | FcγRIII-γ |
| TTP | MyD88 | CD28δ | FcεRIβ |
| TTP | MyD88 | CD28δ | FcεRIγ |
| TTP | MyD88 | CD28δ | DAP10 |
| TTP | MyD88 | CD28δ | DAP12 |
| TTP | MyD88 | CD28δ | CD32 |
| TTP | MyD88 | CD28δ | CD79a |
| TTP | MyD88 | CD28δ | CD79b |
| TTP | MyD88 | CD80 | CD8 |
| TTP | MyD88 | CD80 | CD3ζ |
| TTP | MyD88 | CD80 | CD3δ |
| TTP | MyD88 | CD80 | CD3γ |
| TTP | MyD88 | CD80 | CD3ε |
| TTP | MyD88 | CD80 | FcγRI-γ |
| TTP | MyD88 | CD80 | FcγRIII-γ |
| TTP | MyD88 | CD80 | FcεRIβ |
| TTP | MyD88 | CD80 | FcεRIγ |
| TTP | MyD88 | CD80 | DAP10 |
| TTP | MyD88 | CD80 | DAP12 |
| TTP | MyD88 | CD80 | CD32 |
| TTP | MyD88 | CD80 | CD79a |
| TTP | MyD88 | CD80 | CD79b |
| TTP | MyD88 | CD86 | CD8 |
| TTP | MyD88 | CD86 | CD3ζ |
| TTP | MyD88 | CD86 | CD3δ |
| TTP | MyD88 | CD86 | CD3γ |
| TTP | MyD88 | CD86 | CD3ε |
| TTP | MyD88 | CD86 | FcγRI-γ |
| TTP | MyD88 | CD86 | FcγRIII-γ |
| TTP | MyD88 | CD86 | FcεRIβ |
| TTP | MyD88 | CD86 | FcεRIγ |
| TTP | MyD88 | CD86 | DAP10 |
| TTP | MyD88 | CD86 | DAP12 |
| TTP | MyD88 | CD86 | CD32 |
| TTP | MyD88 | CD86 | CD79a |
| TTP | MyD88 | CD86 | CD79b |
| TTP | MyD88 | OX40 | CD8 |
| TTP | MyD88 | OX40 | CD3ζ |
| TTP | MyD88 | OX40 | CD3δ |
| TTP | MyD88 | OX40 | CD3γ |
| TTP | MyD88 | OX40 | CD3ε |
| TTP | MyD88 | OX40 | FcγRI-γ |
| TTP | MyD88 | OX40 | FcγRIII-γ |
| TTP | MyD88 | OX40 | FcεRIβ |
| TTP | MyD88 | OX40 | FcεRIγ |
| TTP | MyD88 | OX40 | DAP10 |
| TTP | MyD88 | OX40 | DAP12 |
| TTP | MyD88 | OX40 | CD32 |
| TTP | MyD88 | OX40 | CD79a |
| TTP | MyD88 | OX40 | CD79b |
| TTP | MyD88 | DAP10 | CD8 |
| TTP | MyD88 | DAP10 | CD3ζ |
| TTP | MyD88 | DAP10 | CD3δ |
| TTP | MyD88 | DAP10 | CD3γ |
| TTP | MyD88 | DAP10 | CD3ε |
| TTP | MyD88 | DAP10 | FcγRI-γ |
| TTP | MyD88 | DAP10 | FcγRIII-γ |
| TTP | MyD88 | DAP10 | FcεRIβ |
| TTP | MyD88 | DAP10 | FcεRIγ |
| TTP | MyD88 | DAP10 | DAP10 |
| TTP | MyD88 | DAP10 | DAP12 |
| TTP | MyD88 | DAP10 | CD32 |
| TTP | MyD88 | DAP10 | CD79a |
| TTP | MyD88 | DAP10 | CD79b |
| TTP | MyD88 | DAP12 | CD8 |
| TTP | MyD88 | DAP12 | CD3ζ |
| TTP | MyD88 | DAP12 | CD3δ |
| TTP | MyD88 | DAP12 | CD3γ |
| TTP | MyD88 | DAP12 | CD3ε |
| TTP | MyD88 | DAP12 | FcγRI-γ |
| TTP | MyD88 | DAP12 | FcγRIII-γ |
| TTP | MyD88 | DAP12 | FcεRIβ |
| TTP | MyD88 | DAP12 | FcεRIγ |
| TTP | MyD88 | DAP12 | DAP10 |
| TTP | MyD88 | DAP12 | DAP12 |
| TTP | MyD88 | DAP12 | CD32 |
| TTP | MyD88 | DAP12 | CD79a |
| TTP | MyD88 | DAP12 | CD79b |
| TTP | MyD88 | MyD88 | CD8 |
| TTP | MyD88 | MyD88 | CD3ζ |
| TTP | MyD88 | MyD88 | CD3δ |
| TTP | MyD88 | MyD88 | CD3γ |
| TTP | MyD88 | MyD88 | CD3ε |
| TTP | MyD88 | MyD88 | FcγRI-γ |
| TTP | MyD88 | MyD88 | FcγRIII-γ |
| TTP | MyD88 | MyD88 | FcεRIβ |
| TTP | MyD88 | MyD88 | FcεRIγ |
| TTP | MyD88 | MyD88 | DAP10 |
| TTP | MyD88 | MyD88 | DAP12 |
| TTP | MyD88 | MyD88 | CD32 |
| TTP | MyD88 | MyD88 | CD79a |
| TTP | MyD88 | MyD88 | CD79b |
| TTP | MyD88 | CD7 | CD8 |
| TTP | MyD88 | CD7 | CD3ζ |
| TTP | MyD88 | CD7 | CD3δ |
| TTP | MyD88 | CD7 | CD3γ |
| TTP | MyD88 | CD7 | CD3ε |
| TTP | MyD88 | CD7 | FcγRI-γ |
| TTP | MyD88 | CD7 | FcγRIII-γ |
| TTP | MyD88 | CD7 | FcεRIβ |
| TTP | MyD88 | CD7 | FcεRIγ |
| TTP | MyD88 | CD7 | DAP10 |
| TTP | MyD88 | CD7 | DAP12 |
| TTP | MyD88 | CD7 | CD32 |
| TTP | MyD88 | CD7 | CD79a |
| TTP | MyD88 | CD7 | CD79b |
| TTP | MyD88 | BTNL3 | CD8 |
| TTP | MyD88 | BTNL3 | CD3ζ |
| TTP | MyD88 | BTNL3 | CD3δ |
| TTP | MyD88 | BTNL3 | CD3γ |
| TTP | MyD88 | BTNL3 | CD3ε |
| TTP | MyD88 | BTNL3 | FcγRI-γ |
| TTP | MyD88 | BTNL3 | FcγRIII-γ |
| TTP | MyD88 | BTNL3 | FcεRIβ |
| TTP | MyD88 | BTNL3 | FcεRIγ |
| TTP | MyD88 | BTNL3 | DAP10 |
| TTP | MyD88 | BTNL3 | DAP12 |
| TTP | MyD88 | BTNL3 | CD32 |
| TTP | MyD88 | BTNL3 | CD79a |
| TTP | MyD88 | BTNL3 | CD79b |
| TTP | MyD88 | NKG2D | CD8 |
| TTP | MyD88 | NKG2D | CD3ζ |
| TTP | MyD88 | NKG2D | CD3δ |
| TTP | MyD88 | NKG2D | CD3γ |
| TTP | MyD88 | NKG2D | CD3ε |
| TTP | MyD88 | NKG2D | FcγRI-γ |
| TTP | MyD88 | NKG2D | FcγRIII-γ |
| TTP | MyD88 | NKG2D | FcεRIβ |
| TTP | MyD88 | NKG2D | FcεRIγ |
| TTP | MyD88 | NKG2D | DAP10 |
| TTP | MyD88 | NKG2D | DAP12 |
| TTP | MyD88 | NKG2D | CD32 |
| TTP | MyD88 | NKG2D | CD79a |
| TTP | MyD88 | NKG2D | CD79b |
| TTP | CD7 | CD28 | CD8 |
| TTP | CD7 | CD28 | CD3ζ |
| TTP | CD7 | CD28 | CD3δ |
| TTP | CD7 | CD28 | CD3γ |
| TTP | CD7 | CD28 | CD3ε |
| TTP | CD7 | CD28 | FcγRI-γ |
| TTP | CD7 | CD28 | FcγRIII-γ |
| TTP | CD7 | CD28 | FcεRIβ |
| TTP | CD7 | CD28 | FcεRIγ |
| TTP | CD7 | CD28 | DAP10 |
| TTP | CD7 | CD28 | DAP12 |
| TTP | CD7 | CD28 | CD32 |
| TTP | CD7 | CD28 | CD79a |
| TTP | CD7 | CD28 | CD79b |
| TTP | CD7 | CD8 | CD8 |
| TTP | CD7 | CD8 | CD3ζ |
| TTP | CD7 | CD8 | CD3δ |
| TTP | CD7 | CD8 | CD3γ |
| TTP | CD7 | CD8 | CD3ε |
| TTP | CD7 | CD8 | FcγRI-γ |
| TTP | CD7 | CD8 | FcγRIII-γ |
| TTP | CD7 | CD8 | FcεRIβ |
| TTP | CD7 | CD8 | FcεRIγ |
| TTP | CD7 | CD8 | DAP10 |
| TTP | CD7 | CD8 | DAP12 |
| TTP | CD7 | CD8 | CD32 |
| TTP | CD7 | CD8 | CD79a |
| TTP | CD7 | CD8 | CD79b |
| TTP | CD7 | CD4 | CD8 |
| TTP | CD7 | CD4 | CD3ζ |
| TTP | CD7 | CD4 | CD3δ |
| TTP | CD7 | CD4 | CD3γ |
| TTP | CD7 | CD4 | CD3ε |
| TTP | CD7 | CD4 | FcγRI-γ |
| TTP | CD7 | CD4 | FcγRIII-γ |
| TTP | CD7 | CD4 | FcεRIβ |
| TTP | CD7 | CD4 | FcεRIγ |
| TTP | CD7 | CD4 | DAP10 |
| TTP | CD7 | CD4 | DAP12 |
| TTP | CD7 | CD4 | CD32 |
| TTP | CD7 | CD4 | CD79a |
| TTP | CD7 | CD4 | CD79b |
| TTP | CD7 | b2c | CD8 |
| TTP | CD7 | b2c | CD3ζ |
| TTP | CD7 | b2c | CD3δ |
| TTP | CD7 | b2c | CD3γ |
| TTP | CD7 | b2c | CD3ε |
| TTP | CD7 | b2c | FcγRI-γ |
| TTP | CD7 | b2c | FcγRIII-γ |
| TTP | CD7 | b2c | FcεRIβ |
| TTP | CD7 | b2c | FcεRIγ |
| TTP | CD7 | b2c | DAP10 |
| TTP | CD7 | b2c | DAP12 |
| TTP | CD7 | b2c | CD32 |
| TTP | CD7 | b2c | CD79a |
| TTP | CD7 | b2c | CD79b |
| TTP | CD7 | CD137/41BB | CD8 |
| TTP | CD7 | CD137/41BB | CD3ζ |
| TTP | CD7 | CD137/41BB | CD3δ |
| TTP | CD7 | CD137/41BB | CD3γ |
| TTP | CD7 | CD137/41BB | CD3ε |
| TTP | CD7 | CD137/41BB | FcγRI-γ |
| TTP | CD7 | CD137/41BB | FcγRIII-γ |
| TTP | CD7 | CD137/41BB | FcεRIβ |
| TTP | CD7 | CD137/41BB | FcεRIγ |
| TTP | CD7 | CD137/41BB | DAP10 |
| TTP | CD7 | CD137/41BB | DAP12 |
| TTP | CD7 | CD137/41BB | CD32 |
| TTP | CD7 | CD137/41BB | CD79a |
| TTP | CD7 | CD137/41BB | CD79b |
| TTP | CD7 | ICOS | CD8 |
| TTP | CD7 | ICOS | CD3ζ |
| TTP | CD7 | ICOS | CD3δ |
| TTP | CD7 | ICOS | CD3γ |
| TTP | CD7 | ICOS | CD3ε |
| TTP | CD7 | ICOS | FcγRI-γ |
| TTP | CD7 | ICOS | FcγRIII-γ |
| TTP | CD7 | ICOS | FcεRIβ |
| TTP | CD7 | ICOS | FcεRIγ |
| TTP | CD7 | ICOS | DAP10 |
| TTP | CD7 | ICOS | DAP12 |
| TTP | CD7 | ICOS | CD32 |
| TTP | CD7 | ICOS | CD79a |
| TTP | CD7 | ICOS | CD79b |
| TTP | CD7 | CD27 | CD8 |
| TTP | CD7 | CD27 | CD3ζ |
| TTP | CD7 | CD27 | CD3δ |
| TTP | CD7 | CD27 | CD3γ |
| TTP | CD7 | CD27 | CD3ε |
| TTP | CD7 | CD27 | FcγRI-γ |
| TTP | CD7 | CD27 | FcγRIII-γ |
| TTP | CD7 | CD27 | FcεRIβ |
| TTP | CD7 | CD27 | FcεRIγ |
| TTP | CD7 | CD27 | DAP10 |
| TTP | CD7 | CD27 | DAP12 |
| TTP | CD7 | CD27 | CD32 |
| TTP | CD7 | CD27 | CD79a |
| TTP | CD7 | CD27 | CD79b |
| TTP | CD7 | CD28δ | CD8 |
| TTP | CD7 | CD28δ | CD3ζ |
| TTP | CD7 | CD28δ | CD3δ |
| TTP | CD7 | CD28δ | CD3γ |
| TTP | CD7 | CD28δ | CD3ε |
| TTP | CD7 | CD28δ | FcγRI-γ |
| TTP | CD7 | CD28δ | FcγRIII-γ |
| TTP | CD7 | CD28δ | FcεRIβ |
| TTP | CD7 | CD28δ | FcεRIγ |
| TTP | CD7 | CD28δ | DAP10 |
| TTP | CD7 | CD28δ | DAP12 |
| TTP | CD7 | CD28δ | CD32 |
| TTP | CD7 | CD28δ | CD79a |
| TTP | CD7 | CD28δ | CD79b |
| TTP | CD7 | CD80 | CD8 |
| TTP | CD7 | CD80 | CD3ζ |
| TTP | CD7 | CD80 | CD3δ |
| TTP | CD7 | CD80 | CD3γ |
| TTP | CD7 | CD80 | CD3ε |
| TTP | CD7 | CD80 | FcγRI-γ |
| TTP | CD7 | CD80 | FcγRIII-γ |
| TTP | CD7 | CD80 | FcεRIβ |
| TTP | CD7 | CD80 | FcεRIγ |
| TTP | CD7 | CD80 | DAP10 |
| TTP | CD7 | CD80 | DAP12 |
| TTP | CD7 | CD80 | CD32 |
| TTP | CD7 | CD80 | CD79a |
| TTP | CD7 | CD80 | CD79b |
| TTP | CD7 | CD86 | CD8 |
| TTP | CD7 | CD86 | CD3ζ |
| TTP | CD7 | CD86 | CD3δ |
| TTP | CD7 | CD86 | CD3γ |
| TTP | CD7 | CD86 | CD3ε |
| TTP | CD7 | CD86 | FcγRI-γ |
| TTP | CD7 | CD86 | FcγRIII-γ |
| TTP | CD7 | CD86 | FcεRIβ |
| TTP | CD7 | CD86 | FcεRIγ |
| TTP | CD7 | CD86 | DAP10 |
| TTP | CD7 | CD86 | DAP12 |
| TTP | CD7 | CD86 | CD32 |
| TTP | CD7 | CD86 | CD79a |
| TTP | CD7 | CD86 | CD79b |
| TTP | CD7 | OX40 | CD8 |
| TTP | CD7 | OX40 | CD3ζ |
| TTP | CD7 | OX40 | CD3δ |
| TTP | CD7 | OX40 | CD3γ |
| TTP | CD7 | OX40 | CD3ε |
| TTP | CD7 | OX40 | FcγRI-γ |
| TTP | CD7 | OX40 | FcγRIII-γ |
| TTP | CD7 | OX40 | FcεRIβ |
| TTP | CD7 | OX40 | FcεRIγ |
| TTP | CD7 | OX40 | DAP10 |
| TTP | CD7 | OX40 | DAP12 |
| TTP | CD7 | OX40 | CD32 |
| TTP | CD7 | OX40 | CD79a |
| TTP | CD7 | OX40 | CD79b |
| TTP | CD7 | DAP10 | CD8 |
| TTP | CD7 | DAP10 | CD3ζ |
| TTP | CD7 | DAP10 | CD3δ |
| TTP | CD7 | DAP10 | CD3γ |
| TTP | CD7 | DAP10 | CD3ε |
| TTP | CD7 | DAP10 | FcγRI-γ |
| TTP | CD7 | DAP10 | FcγRIII-γ |
| TTP | CD7 | DAP10 | FcεRIβ |
| TTP | CD7 | DAP10 | FcεRIγ |
| TTP | CD7 | DAP10 | DAP10 |
| TTP | CD7 | DAP10 | DAP12 |
| TTP | CD7 | DAP10 | CD32 |
| TTP | CD7 | DAP10 | CD79a |
| TTP | CD7 | DAP10 | CD79b |
| TTP | CD7 | DAP12 | CD8 |
| TTP | CD7 | DAP12 | CD3ζ |
| TTP | CD7 | DAP12 | CD3δ |
| TTP | CD7 | DAP12 | CD3γ |
| TTP | CD7 | DAP12 | CD3ε |
| TTP | CD7 | DAP12 | FcγRI-γ |
| TTP | CD7 | DAP12 | FcγRIII-γ |
| TTP | CD7 | DAP12 | FcεRIβ |
| TTP | CD7 | DAP12 | FcεRIγ |
| TTP | CD7 | DAP12 | DAP10 |
| TTP | CD7 | DAP12 | DAP12 |
| TTP | CD7 | DAP12 | CD32 |
| TTP | CD7 | DAP12 | CD79a |
| TTP | CD7 | DAP12 | CD79b |
| TTP | CD7 | MyD88 | CD8 |
| TTP | CD7 | MyD88 | CD3ζ |
| TTP | CD7 | MyD88 | CD3δ |
| TTP | CD7 | MyD88 | CD3γ |
| TTP | CD7 | MyD88 | CD3ε |
| TTP | CD7 | MyD88 | FcγRI-γ |
| TTP | CD7 | MyD88 | FcγRIII-γ |
| TTP | CD7 | MyD88 | FcεRIβ |
| TTP | CD7 | MyD88 | FcεRIγ |
| TTP | CD7 | MyD88 | DAP10 |
| TTP | CD7 | MyD88 | DAP12 |
| TTP | CD7 | MyD88 | CD32 |
| TTP | CD7 | MyD88 | CD79a |
| TTP | CD7 | MyD88 | CD79b |
| TTP | CD7 | CD7 | CD8 |
| TTP | CD7 | CD7 | CD3ζ |
| TTP | CD7 | CD7 | CD3δ |
| TTP | CD7 | CD7 | CD3γ |
| TTP | CD7 | CD7 | CD3ε |
| TTP | CD7 | CD7 | FcγRI-γ |
| TTP | CD7 | CD7 | FcγRIII-γ |
| TTP | CD7 | CD7 | FcεRIβ |
| TTP | CD7 | CD7 | FcεRIγ |
| TTP | CD7 | CD7 | DAP10 |
| TTP | CD7 | CD7 | DAP12 |
| TTP | CD7 | CD7 | CD32 |
| TTP | CD7 | CD7 | CD79a |
| TTP | CD7 | CD7 | CD79b |
| TTP | CD7 | BTNL3 | CD8 |
| TTP | CD7 | BTNL3 | CD3ζ |
| TTP | CD7 | BTNL3 | CD3δ |
| TTP | CD7 | BTNL3 | CD3γ |
| TTP | CD7 | BTNL3 | CD3ε |
| TTP | CD7 | BTNL3 | FcγRI-γ |
| TTP | CD7 | BTNL3 | FcγRIII-γ |
| TTP | CD7 | BTNL3 | FcεRIβ |
| TTP | CD7 | BTNL3 | FcεRIγ |
| TTP | CD7 | BTNL3 | DAP10 |
| TTP | CD7 | BTNL3 | DAP12 |
| TTP | CD7 | BTNL3 | CD32 |
| TTP | CD7 | BTNL3 | CD79a |
| TTP | CD7 | BTNL3 | CD79b |
| TTP | CD7 | NKG2D | CD8 |
| TTP | CD7 | NKG2D | CD3ζ |
| TTP | CD7 | NKG2D | CD3δ |
| TTP | CD7 | NKG2D | CD3γ |
| TTP | CD7 | NKG2D | CD3ε |
| TTP | CD7 | NKG2D | FcγRI-γ |
| TTP | CD7 | NKG2D | FcγRIII-γ |
| TTP | CD7 | NKG2D | FcεRIβ |
| TTP | CD7 | NKG2D | FcεRIγ |
| TTP | CD7 | NKG2D | DAP10 |
| TTP | CD7 | NKG2D | DAP12 |
| TTP | CD7 | NKG2D | CD32 |
| TTP | CD7 | NKG2D | CD79a |
| TTP | CD7 | NKG2D | CD79b |
| TTP | BTNL3 | CD28 | CD8 |
| TTP | BTNL3 | CD28 | CD3ζ |
| TTP | BTNL3 | CD28 | CD3δ |
| TTP | BTNL3 | CD28 | CD3γ |
| TTP | BTNL3 | CD28 | CD3ε |
| TTP | BTNL3 | CD28 | FcγRI-γ |
| TTP | BTNL3 | CD28 | FcγRIII-γ |
| TTP | BTNL3 | CD28 | FcεRIβ |
| TTP | BTNL3 | CD28 | FcεRIγ |
| TTP | BTNL3 | CD28 | DAP10 |
| TTP | BTNL3 | CD28 | DAP12 |
| TTP | BTNL3 | CD28 | CD32 |
| TTP | BTNL3 | CD28 | CD79a |
| TTP | BTNL3 | CD28 | CD79b |
| TTP | BTNL3 | CD8 | CD8 |
| TTP | BTNL3 | CD8 | CD3ζ |
| TTP | BTNL3 | CD8 | CD3δ |
| TTP | BTNL3 | CD8 | CD3γ |
| TTP | BTNL3 | CD8 | CD3ε |
| TTP | BTNL3 | CD8 | FcγRI-γ |
| TTP | BTNL3 | CD8 | FcγRIII-γ |
| TTP | BTNL3 | CD8 | FcεRIβ |
| TTP | BTNL3 | CD8 | FcεRIγ |
| TTP | BTNL3 | CD8 | DAP10 |
| TTP | BTNL3 | CD8 | DAP12 |
| TTP | BTNL3 | CD8 | CD32 |
| TTP | BTNL3 | CD8 | CD79a |
| TTP | BTNL3 | CD8 | CD79b |
| TTP | BTNL3 | CD4 | CD8 |
| TTP | BTNL3 | CD4 | CD3ζ |
| TTP | BTNL3 | CD4 | CD3δ |
| TTP | BTNL3 | CD4 | CD3γ |
| TTP | BTNL3 | CD4 | CD3ε |
| TTP | BTNL3 | CD4 | FcγRI-γ |
| TTP | BTNL3 | CD4 | FcγRIII-γ |
| TTP | BTNL3 | CD4 | FcεRIβ |
| TTP | BTNL3 | CD4 | FcεRIγ |
| TTP | BTNL3 | CD4 | DAP10 |
| TTP | BTNL3 | CD4 | DAP12 |
| TTP | BTNL3 | CD4 | CD32 |
| TTP | BTNL3 | CD4 | CD79a |
| TTP | BTNL3 | CD4 | CD79b |
| TTP | BTNL3 | b2c | CD8 |
| TTP | BTNL3 | b2c | CD3ζ |
| TTP | BTNL3 | b2c | CD3δ |
| TTP | BTNL3 | b2c | CD3γ |
| TTP | BTNL3 | b2c | CD3ε |
| TTP | BTNL3 | b2c | FcγRI-γ |
| TTP | BTNL3 | b2c | FcγRIII-γ |
| TTP | BTNL3 | b2c | FcεRIβ |
| TTP | BTNL3 | b2c | FcεRIγ |
| TTP | BTNL3 | b2c | DAP10 |
| TTP | BTNL3 | b2c | DAP12 |
| TTP | BTNL3 | b2c | CD32 |
| TTP | BTNL3 | b2c | CD79a |
| TTP | BTNL3 | b2c | CD79b |
| TTP | BTNL3 | CD137/41BB | CD8 |
| TTP | BTNL3 | CD137/41BB | CD3ζ |
| TTP | BTNL3 | CD137/41BB | CD3δ |
| TTP | BTNL3 | CD137/41BB | CD3γ |
| TTP | BTNL3 | CD137/41BB | CD3ε |
| TTP | BTNL3 | CD137/41BB | FcγRI-γ |
| TTP | BTNL3 | CD137/41BB | FcγRIII-γ |
| TTP | BTNL3 | CD137/41BB | FcεRIβ |
| TTP | BTNL3 | CD137/41BB | FcεRIγ |
| TTP | BTNL3 | CD137/41BB | DAP10 |
| TTP | BTNL3 | CD137/41BB | DAP12 |
| TTP | BTNL3 | CD137/41BB | CD32 |
| TTP | BTNL3 | CD137/41BB | CD79a |
| TTP | BTNL3 | CD137/41BB | CD79b |
| TTP | BTNL3 | ICOS | CD8 |
| TTP | BTNL3 | ICOS | CD3ζ |
| TTP | BTNL3 | ICOS | CD3δ |
| TTP | BTNL3 | ICOS | CD3γ |
| TTP | BTNL3 | ICOS | CD3ε |
| TTP | BTNL3 | ICOS | FcγRI-γ |
| TTP | BTNL3 | ICOS | FcγRIII-γ |
| TTP | BTNL3 | ICOS | FcεRIβ |
| TTP | BTNL3 | ICOS | FcεRIγ |
| TTP | BTNL3 | ICOS | DAP10 |
| TTP | BTNL3 | ICOS | DAP12 |
| TTP | BTNL3 | ICOS | CD32 |
| TTP | BTNL3 | ICOS | CD79a |
| TTP | BTNL3 | ICOS | CD79b |
| TTP | BTNL3 | CD27 | CD8 |
| TTP | BTNL3 | CD27 | CD3ζ |
| TTP | BTNL3 | CD27 | CD3δ |
| TTP | BTNL3 | CD27 | CD3γ |
| TTP | BTNL3 | CD27 | CD3ε |
| TTP | BTNL3 | CD27 | FcγRI-γ |
| TTP | BTNL3 | CD27 | FcγRIII-γ |
| TTP | BTNL3 | CD27 | FcεRIβ |
| TTP | BTNL3 | CD27 | FcεRIγ |
| TTP | BTNL3 | CD27 | DAP10 |
| TTP | BTNL3 | CD27 | DAP12 |
| TTP | BTNL3 | CD27 | CD32 |
| TTP | BTNL3 | CD27 | CD79a |
| TTP | BTNL3 | CD27 | CD79b |
| TTP | BTNL3 | CD28δ | CD8 |
| TTP | BTNL3 | CD28δ | CD3ζ |
| TTP | BTNL3 | CD28δ | CD3δ |
| TTP | BTNL3 | CD28δ | CD3γ |
| TTP | BTNL3 | CD28δ | CD3ε |
| TTP | BTNL3 | CD28δ | FcγRI-γ |
| TTP | BTNL3 | CD28δ | FcγRIII-γ |
| TTP | BTNL3 | CD28δ | FcεRIβ |
| TTP | BTNL3 | CD28δ | FcεRIγ |
| TTP | BTNL3 | CD28δ | DAP10 |
| TTP | BTNL3 | CD28δ | DAP12 |
| TTP | BTNL3 | CD28δ | CD32 |
| TTP | BTNL3 | CD28δ | CD79a |
| TTP | BTNL3 | CD28δ | CD79b |
| TTP | BTNL3 | CD80 | CD8 |
| TTP | BTNL3 | CD80 | CD3ζ |
| TTP | BTNL3 | CD80 | CD3δ |
| TTP | BTNL3 | CD80 | CD3γ |
| TTP | BTNL3 | CD80 | CD3ε |
| TTP | BTNL3 | CD80 | FcγRI-γ |
| TTP | BTNL3 | CD80 | FcγRIII-γ |
| TTP | BTNL3 | CD80 | FcεRIβ |
| TTP | BTNL3 | CD80 | FcεRIγ |
| TTP | BTNL3 | CD80 | DAP10 |
| TTP | BTNL3 | CD80 | DAP12 |
| TTP | BTNL3 | CD80 | CD32 |
| TTP | BTNL3 | CD80 | CD79a |
| TTP | BTNL3 | CD80 | CD79b |
| TTP | BTNL3 | CD86 | CD8 |
| TTP | BTNL3 | CD86 | CD3ζ |
| TTP | BTNL3 | CD86 | CD3δ |
| TTP | BTNL3 | CD86 | CD3γ |
| TTP | BTNL3 | CD86 | CD3ε |
| TTP | BTNL3 | CD86 | FcγRI-γ |
| TTP | BTNL3 | CD86 | FcγRIII-γ |
| TTP | BTNL3 | CD86 | FcεRIβ |
| TTP | BTNL3 | CD86 | FcεRIγ |
| TTP | BTNL3 | CD86 | DAP10 |
| TTP | BTNL3 | CD86 | DAP12 |
| TTP | BTNL3 | CD86 | CD32 |
| TTP | BTNL3 | CD86 | CD79a |
| TTP | BTNL3 | CD86 | CD79b |
| TTP | BTNL3 | OX40 | CD8 |
| TTP | BTNL3 | OX40 | CD3ζ |
| TTP | BTNL3 | OX40 | CD3δ |
| TTP | BTNL3 | OX40 | CD3γ |
| TTP | BTNL3 | OX40 | CD3ε |
| TTP | BTNL3 | OX40 | FcγRI-γ |
| TTP | BTNL3 | OX40 | FcγRIII-γ |
| TTP | BTNL3 | OX40 | FcεRIβ |
| TTP | BTNL3 | OX40 | FcεRIγ |
| TTP | BTNL3 | OX40 | DAP10 |
| TTP | BTNL3 | OX40 | DAP12 |
| TTP | BTNL3 | OX40 | CD32 |
| TTP | BTNL3 | OX40 | CD79a |
| TTP | BTNL3 | OX40 | CD79b |
| TTP | BTNL3 | DAP10 | CD8 |
| TTP | BTNL3 | DAP10 | CD3ζ |
| TTP | BTNL3 | DAP10 | CD3δ |
| TTP | BTNL3 | DAP10 | CD3γ |
| TTP | BTNL3 | DAP10 | CD3ε |
| TTP | BTNL3 | DAP10 | FcγRI-γ |
| TTP | BTNL3 | DAP10 | FcγRIII-γ |
| TTP | BTNL3 | DAP10 | FcεRIβ |
| TTP | BTNL3 | DAP10 | FcεRIγ |
| TTP | BTNL3 | DAP10 | DAP10 |
| TTP | BTNL3 | DAP10 | DAP12 |
| TTP | BTNL3 | DAP10 | CD32 |
| TTP | BTNL3 | DAP10 | CD79a |
| TTP | BTNL3 | DAP10 | CD79b |
| TTP | BTNL3 | DAP12 | CD8 |
| TTP | BTNL3 | DAP12 | CD3ζ |
| TTP | BTNL3 | DAP12 | CD3δ |
| TTP | BTNL3 | DAP12 | CD3γ |
| TTP | BTNL3 | DAP12 | CD3ε |
| TTP | BTNL3 | DAP12 | FcγRI-γ |
| TTP | BTNL3 | DAP12 | FcγRIII-γ |
| TTP | BTNL3 | DAP12 | FcεRIβ |
| TTP | BTNL3 | DAP12 | FcεRIγ |
| TTP | BTNL3 | DAP12 | DAP10 |
| TTP | BTNL3 | DAP12 | DAP12 |
| TTP | BTNL3 | DAP12 | CD32 |
| TTP | BTNL3 | DAP12 | CD79a |
| TTP | BTNL3 | DAP12 | CD79b |
| TTP | BTNL3 | MyD88 | CD8 |
| TTP | BTNL3 | MyD88 | CD3ζ |
| TTP | BTNL3 | MyD88 | CD3δ |
| TTP | BTNL3 | MyD88 | CD3γ |
| TTP | BTNL3 | MyD88 | CD3ε |
| TTP | BTNL3 | MyD88 | FcγRI-γ |
| TTP | BTNL3 | MyD88 | FcγRIII-γ |
| TTP | BTNL3 | MyD88 | FcεRIβ |
| TTP | BTNL3 | MyD88 | FcεRIγ |
| TTP | BTNL3 | MyD88 | DAP10 |
| TTP | BTNL3 | MyD88 | DAP12 |
| TTP | BTNL3 | MyD88 | CD32 |
| TTP | BTNL3 | MyD88 | CD79a |
| TTP | BTNL3 | MyD88 | CD79b |
| TTP | BTNL3 | CD7 | CD8 |
| TTP | BTNL3 | CD7 | CD3ζ |
| TTP | BTNL3 | CD7 | CD3δ |
| TTP | BTNL3 | CD7 | CD3γ |
| TTP | BTNL3 | CD7 | CD3ε |
| TTP | BTNL3 | CD7 | FcγRI-γ |
| TTP | BTNL3 | CD7 | FcγRIII-γ |
| TTP | BTNL3 | CD7 | FcεRIβ |
| TTP | BTNL3 | CD7 | FcεRIγ |
| TTP | BTNL3 | CD7 | DAP10 |
| TTP | BTNL3 | CD7 | DAP12 |
| TTP | BTNL3 | CD7 | CD32 |
| TTP | BTNL3 | CD7 | CD79a |
| TTP | BTNL3 | CD7 | CD79b |
| TTP | BTNL3 | BTNL3 | CD8 |
| TTP | BTNL3 | BTNL3 | CD3ζ |
| TTP | BTNL3 | BTNL3 | CD3δ |
| TTP | BTNL3 | BTNL3 | CD3γ |
| TTP | BTNL3 | BTNL3 | CD3ε |
| TTP | BTNL3 | BTNL3 | FcγRI-γ |
| TTP | BTNL3 | BTNL3 | FcγRIII-γ |
| TTP | BTNL3 | BTNL3 | FcεRIβ |
| TTP | BTNL3 | BTNL3 | FcεRIγ |
| TTP | BTNL3 | BTNL3 | DAP10 |
| TTP | BTNL3 | BTNL3 | DAP12 |
| TTP | BTNL3 | BTNL3 | CD32 |
| TTP | BTNL3 | BTNL3 | CD79a |
| TTP | BTNL3 | BTNL3 | CD79b |
| TTP | BTNL3 | NKG2D | CD8 |
| TTP | BTNL3 | NKG2D | CD3ζ |
| TTP | BTNL3 | NKG2D | CD3δ |
| TTP | BTNL3 | NKG2D | CD3γ |
| TTP | BTNL3 | NKG2D | CD3ε |
| TTP | BTNL3 | NKG2D | FcγRI-γ |
| TTP | BTNL3 | NKG2D | FcγRIII-γ |
| TTP | BTNL3 | NKG2D | FcεRIβ |
| TTP | BTNL3 | NKG2D | FcεRIγ |
| TTP | BTNL3 | NKG2D | DAP10 |
| TTP | BTNL3 | NKG2D | DAP12 |
| TTP | BTNL3 | NKG2D | CD32 |
| TTP | BTNL3 | NKG2D | CD79a |
| TTP | BTNL3 | NKG2D | CD79b |
| TTP | NKG2D | CD28 | CD8 |
| TTP | NKG2D | CD28 | CD3ζ |
| TTP | NKG2D | CD28 | CD3δ |
| TTP | NKG2D | CD28 | CD3γ |
| TTP | NKG2D | CD28 | CD3ε |
| TTP | NKG2D | CD28 | FcγRI-γ |
| TTP | NKG2D | CD28 | FcγRIII-γ |
| TTP | NKG2D | CD28 | FcεRIβ |
| TTP | NKG2D | CD28 | FcεRIγ |
| TTP | NKG2D | CD28 | DAP10 |
| TTP | NKG2D | CD28 | DAP12 |
| TTP | NKG2D | CD28 | CD32 |
| TTP | NKG2D | CD28 | CD79a |
| TTP | NKG2D | CD28 | CD79b |
| TTP | NKG2D | CD8 | CD8 |
| TTP | NKG2D | CD8 | CD3ζ |
| TTP | NKG2D | CD8 | CD3δ |
| TTP | NKG2D | CD8 | CD3γ |
| TTP | NKG2D | CD8 | CD3ε |
| TTP | NKG2D | CD8 | FcγRI-γ |
| TTP | NKG2D | CD8 | FcγRIII-γ |
| TTP | NKG2D | CD8 | FcεRIβ |
| TTP | NKG2D | CD8 | FcεRIγ |
| TTP | NKG2D | CD8 | DAP10 |
| TTP | NKG2D | CD8 | DAP12 |
| TTP | NKG2D | CD8 | CD32 |
| TTP | NKG2D | CD8 | CD79a |
| TTP | NKG2D | CD8 | CD79b |
| TTP | NKG2D | CD4 | CD8 |
| TTP | NKG2D | CD4 | CD3ζ |
| TTP | NKG2D | CD4 | CD3δ |
| TTP | NKG2D | CD4 | CD3γ |
| TTP | NKG2D | CD4 | CD3ε |
| TTP | NKG2D | CD4 | FcγRI-γ |
| TTP | NKG2D | CD4 | FcγRIII-γ |
| TTP | NKG2D | CD4 | FcεRIβ |
| TTP | NKG2D | CD4 | FcεRIγ |
| TTP | NKG2D | CD4 | DAP10 |
| TTP | NKG2D | CD4 | DAP12 |
| TTP | NKG2D | CD4 | CD32 |
| TTP | NKG2D | CD4 | CD79a |
| TTP | NKG2D | CD4 | CD79b |
| TTP | NKG2D | b2c | CD8 |
| TTP | NKG2D | b2c | CD3ζ |
| TTP | NKG2D | b2c | CD3δ |
| TTP | NKG2D | b2c | CD3γ |
| TTP | NKG2D | b2c | CD3ε |
| TTP | NKG2D | b2c | FcγRI-γ |
| TTP | NKG2D | b2c | FcγRIII-γ |
| TTP | NKG2D | b2c | FcεRIβ |
| TTP | NKG2D | b2c | FcεRIγ |
| TTP | NKG2D | b2c | DAP10 |
| TTP | NKG2D | b2c | DAP12 |
| TTP | NKG2D | b2c | CD32 |
| TTP | NKG2D | b2c | CD79a |
| TTP | NKG2D | b2c | CD79b |
| TTP | NKG2D | CD137/41BB | CD8 |
| TTP | NKG2D | CD137/41BB | CD3ζ |
| TTP | NKG2D | CD137/41BB | CD3δ |
| TTP | NKG2D | CD137/41BB | CD3γ |
| TTP | NKG2D | CD137/41BB | CD3ε |
| TTP | NKG2D | CD137/41BB | FcγRI-γ |
| TTP | NKG2D | CD137/41BB | FcγRIII-γ |
| TTP | NKG2D | CD137/41BB | FcεRIβ |
| TTP | NKG2D | CD137/41BB | FcεRIγ |
| TTP | NKG2D | CD137/41BB | DAP10 |
| TTP | NKG2D | CD137/41BB | DAP12 |
| TTP | NKG2D | CD137/41BB | CD32 |
| TTP | NKG2D | CD137/41BB | CD79a |
| TTP | NKG2D | CD137/41BB | CD79b |
| TTP | NKG2D | ICOS | CD8 |
| TTP | NKG2D | ICOS | CD3ζ |
| TTP | NKG2D | ICOS | CD3δ |
| TTP | NKG2D | ICOS | CD3γ |
| TTP | NKG2D | ICOS | CD3ε |
| TTP | NKG2D | ICOS | FcγRI-γ |
| TTP | NKG2D | ICOS | FcγRIII-γ |
| TTP | NKG2D | ICOS | FcεRIβ |
| TTP | NKG2D | ICOS | FcεRIγ |
| TTP | NKG2D | ICOS | DAP10 |
| TTP | NKG2D | ICOS | DAP12 |
| TTP | NKG2D | ICOS | CD32 |
| TTP | NKG2D | ICOS | CD79a |
| TTP | NKG2D | ICOS | CD79b |
| TTP | NKG2D | CD27 | CD8 |
| TTP | NKG2D | CD27 | CD3ζ |
| TTP | NKG2D | CD27 | CD3δ |
| TTP | NKG2D | CD27 | CD3γ |
| TTP | NKG2D | CD27 | CD3ε |
| TTP | NKG2D | CD27 | FcγRI-γ |
| TTP | NKG2D | CD27 | FcγRIII-γ |
| TTP | NKG2D | CD27 | FcεRIβ |
| TTP | NKG2D | CD27 | FcεRIγ |
| TTP | NKG2D | CD27 | DAP10 |
| TTP | NKG2D | CD27 | DAP12 |
| TTP | NKG2D | CD27 | CD32 |
| TTP | NKG2D | CD27 | CD79a |
| TTP | NKG2D | CD27 | CD79b |
| TTP | NKG2D | CD28δ | CD8 |
| TTP | NKG2D | CD28δ | CD3ζ |
| TTP | NKG2D | CD28δ | CD3δ |
| TTP | NKG2D | CD28δ | CD3γ |
| TTP | NKG2D | CD28δ | CD3ε |
| TTP | NKG2D | CD28δ | FcγRI-γ |
| TTP | NKG2D | CD28δ | FcγRIII-γ |
| TTP | NKG2D | CD28δ | FcεRIβ |
| TTP | NKG2D | CD28δ | FcεRIγ |
| TTP | NKG2D | CD28δ | DAP10 |
| TTP | NKG2D | CD28δ | DAP12 |
| TTP | NKG2D | CD28δ | CD32 |
| TTP | NKG2D | CD28δ | CD79a |
| TTP | NKG2D | CD28δ | CD79b |
| TTP | NKG2D | CD8 | CD80 |
| TTP | NKG2D | CD80 | CD3ζ |
| TTP | NKG2D | CD80 | CD3δ |
| TTP | NKG2D | CD80 | CD3γ |
| TTP | NKG2D | CD80 | CD3ε |
| TTP | NKG2D | CD80 | FcγRI-γ |
| TTP | NKG2D | CD80 | FcγRIII-γ |
| TTP | NKG2D | CD80 | FcεRIβ |
| TTP | NKG2D | CD80 | FcεRIγ |
| TTP | NKG2D | CD80 | DAP10 |
| TTP | NKG2D | CD80 | DAP12 |
| TTP | NKG2D | CD80 | CD32 |
| TTP | NKG2D | CD80 | CD79a |
| TTP | NKG2D | CD80 | CD79b |
| TTP | NKG2D | CD86 | CD8 |
| TTP | NKG2D | CD86 | CD3ζ |
| TTP | NKG2D | CD86 | CD3δ |
| TTP | NKG2D | CD86 | CD3γ |
| TTP | NKG2D | CD86 | CD3ε |
| TTP | NKG2D | CD86 | FcγRI-γ |
| TTP | NKG2D | CD86 | FcγRIII-γ |
| TTP | NKG2D | CD86 | FcεRIβ |
| TTP | NKG2D | CD86 | FcεRIγ |
| TTP | NKG2D | CD86 | DAP10 |
| TTP | NKG2D | CD86 | DAP12 |
| TTP | NKG2D | CD86 | CD32 |
| TTP | NKG2D | CD86 | CD79a |
| TTP | NKG2D | CD86 | CD79b |
| TTP | NKG2D | OX40 | CD8 |
| TTP | NKG2D | OX40 | CD3ζ |
| TTP | NKG2D | OX40 | CD3δ |
| TTP | NKG2D | OX40 | CD3γ |
| TTP | NKG2D | OX40 | CD3ε |
| TTP | NKG2D | OX40 | FcγRI-γ |
| TTP | NKG2D | OX40 | FcγRIII-γ |
| TTP | NKG2D | OX40 | FcεRIβ |
| TTP | NKG2D | OX40 | FcεRIγ |
| TTP | NKG2D | OX40 | DAP10 |
| TTP | NKG2D | OX40 | DAP12 |
| TTP | NKG2D | OX40 | CD32 |
| TTP | NKG2D | OX40 | CD79a |
| TTP | NKG2D | OX40 | CD79b |
| TTP | NKG2D | DAP10 | CD8 |
| TTP | NKG2D | DAP10 | CD3ζ |
| TTP | NKG2D | DAP10 | CD3δ |
| TTP | NKG2D | DAP10 | CD3γ |
| TTP | NKG2D | DAP10 | CD3ε |
| TTP | NKG2D | DAP10 | FcγRI-γ |
| TTP | NKG2D | DAP10 | FcγRIII-γ |
| TTP | NKG2D | DAP10 | FcεRIβ |
| TTP | NKG2D | DAP10 | FcεRIγ |
| TTP | NKG2D | DAP10 | DAP10 |
| TTP | NKG2D | DAP10 | DAP12 |
| TTP | NKG2D | DAP10 | CD32 |
| TTP | NKG2D | DAP10 | CD79a |
| TTP | NKG2D | DAP10 | CD79b |
| TTP | NKG2D | DAP12 | CD8 |
| TTP | NKG2D | DAP12 | CD3ζ |
| TTP | NKG2D | DAP12 | CD3δ |
| TTP | NKG2D | DAP12 | CD3γ |
| TTP | NKG2D | DAP12 | CD3ε |
| TTP | NKG2D | DAP12 | FcγRI-γ |
| TTP | NKG2D | DAP12 | FcγRIII-γ |
| TTP | NKG2D | DAP12 | FcεRIβ |
| TTP | NKG2D | DAP12 | FcεRIγ |
| TTP | NKG2D | DAP12 | DAP10 |
| TTP | NKG2D | DAP12 | DAP12 |
| TTP | NKG2D | DAP12 | CD32 |
| TTP | NKG2D | DAP12 | CD79a |
| TTP | NKG2D | DAP12 | CD79b |
| TTP | NKG2D | MyD88 | CD8 |
| TTP | NKG2D | MyD88 | CD3ζ |
| TTP | NKG2D | MyD88 | CD3δ |
| TTP | NKG2D | MyD88 | CD3γ |
| TTP | NKG2D | MyD88 | CD3ε |
| TTP | NKG2D | MyD88 | FcγRI-γ |
| TTP | NKG2D | MyD88 | FcγRIII-γ |
| TTP | NKG2D | MyD88 | FcεRIβ |
| TTP | NKG2D | MyD88 | FcεRIγ |
| TTP | NKG2D | MyD88 | DAP10 |
| TTP | NKG2D | MyD88 | DAP12 |
| TTP | NKG2D | MyD88 | CD32 |
| TTP | NKG2D | MyD88 | CD79a |
| TTP | NKG2D | MyD88 | CD79b |
| TTP | NKG2D | CD7 | CD8 |
| TTP | NKG2D | CD7 | CD3ζ |
| TTP | NKG2D | CD7 | CD3δ |
| TTP | NKG2D | CD7 | CD3γ |
| TTP | NKG2D | CD7 | CD3ε |
| TTP | NKG2D | CD7 | FcγRI-γ |
| TTP | NKG2D | CD7 | FcγRIII-γ |
| TTP | NKG2D | CD7 | FcεRIβ |
| TTP | NKG2D | CD7 | FcεRIγ |
| TTP | NKG2D | CD7 | DAP10 |
| TTP | NKG2D | CD7 | DAP12 |
| TTP | NKG2D | CD7 | CD32 |
| TTP | NKG2D | CD7 | CD79a |
| TTP | NKG2D | CD7 | CD79b |
| TTP | NKG2D | BTNL3 | CD8 |
| TTP | NKG2D | BTNL3 | CD3ζ |
| TTP | NKG2D | BTNL3 | CD3δ |
| TTP | NKG2D | BTNL3 | CD3γ |
| TTP | NKG2D | BTNL3 | CD3ε |
| TTP | NKG2D | BTNL3 | FcγRI-γ |
| TTP | NKG2D | BTNL3 | FcγRIII-γ |
| TTP | NKG2D | BTNL3 | FcεRIβ |
| TTP | NKG2D | BTNL3 | FcεRIγ |
| TTP | NKG2D | BTNL3 | DAP10 |
| TTP | NKG2D | BTNL3 | DAP12 |
| TTP | NKG2D | BTNL3 | CD32 |
| TTP | NKG2D | BTNL3 | CD79a |
| TTP | NKG2D | BTNL3 | CD79b |
| TTP | NKG2D | NKG2D | CD8 |
| TTP | NKG2D | NKG2D | CD3ζ |
| TTP | NKG2D | NKG2D | CD3δ |
| TTP | NKG2D | NKG2D | CD3γ |
| TTP | NKG2D | NKG2D | CD3ε |
| TTP | NKG2D | NKG2D | FcγRI-γ |
| TTP | NKG2D | NKG2D | FcγRIII-γ |
| TTP | NKG2D | NKG2D | FcεRIβ |
| TTP | NKG2D | NKG2D | FcεRIγ |
| TTP | NKG2D | NKG2D | DAP10 |
| TTP | NKG2D | NKG2D | DAP12 |
| TTP | NKG2D | NKG2D | CD32 |
| TTP | NKG2D | NKG2D | CD79a |
| TTP | NKG2D | NKG2D | CD79b |

TABLE 4
CARs lacking Co-Simulatory Signal (for dual CAR approach)

| | Co-stimulatory Signal | Signal Domain |
|---|---|---|
| TTP | none | CD8 |
| TTP | none | CD3ζ |
| TTP | none | CD3δ |
| TTP | none | CD3γ |
| TTP | none | CD3ε |
| TTP | none | FcγRI-γ |
| TTP | none | FcγRIII-γ |
| TTP | none | FcεRIβ |
| TTP | none | FcεRIγ |
| TTP | none | DAP10 |
| TTP | none | DAP12 |
| TTP | none | CD32 |
| TTP | none | CD79a |
| TTP | none | CD8 |
| TTP | none | CD3ζ |
| TTP | none | CD3δ |
| TTP | none | CD3γ |
| TTP | none | CD3ε |
| TTP | none | FcγRI-γ |

TABLE 5
CARs lacking Signal Domain (for dual CAR approach)

| | Co-stimulatory Signal | Signal Domain |
|---|---|---|
| TTP | CD28 | none |
| TTP | CD8 | none |
| TTP | CD4 | none |
| TTP | b2c | none |
| TTP | CD137/41BB | none |
| TTP | ICOS | none |
| TTP | CD27 | none |
| TTP | CD28δ | none |
| TTP | CD80 | none |
| TTP | CD86 | none |
| TTP | OX40 | none |
| TTP | DAP10 | none |
| TTP | MyD88 | none |
| TTP | CD7 | none |
| TTP | DAP12 | none |
| TTP | MyD88 | none |
| TTP | CD7 | none |
| TTP | BTNL3 | none |
| TTP | NKG2D | none |

TABLE 6
Third Generation CARs lacking Signal Domain
(for dual CAR approach)

| | Co-stimulatory | Co-stimulatory | Signal |
|---|---|---|---|
| | Signal | Signal | Domain |
| TTP | CD28 | CD28 | none |
| TTP | CD28 | CD8 | none |
| TTP | CD28 | CD4 | none |
| TTP | CD28 | b2c | none |
| TTP | CD28 | CD137/41BB | none |
| TTP | CD28 | ICOS | none |
| TTP | CD28 | CD27 | none |
| TTP | CD28 | CD28δ | none |
| TTP | CD28 | CD80 | none |
| TTP | CD28 | CD86 | none |
| TTP | CD28 | OX40 | none |
| TTP | CD28 | DAP10 | none |
| TTP | CD28 | MyD88 | none |
| TTP | CD28 | CD7 | none |
| TTP | CD28 | DAP12 | none |
| TTP | CD28 | MyD88 | none |
| TTP | CD28 | CD7 | none |
| TTP | CD8 | CD28 | none |
| TTP | CD8 | CD8 | none |
| TTP | CD8 | CD4 | none |
| TTP | CD8 | b2c | none |
| TTP | CD8 | CD137/41BB | none |
| TTP | CD8 | ICOS | none |
| TTP | CD8 | CD27 | none |
| TTP | CD8 | CD28δ | none |
| TTP | CD8 | CD80 | none |
| TTP | CD8 | CD86 | none |
| TTP | CD8 | OX40 | none |
| TTP | CD8 | DAP10 | none |
| TTP | CD8 | MyD88 | none |
| TTP | CD8 | CD7 | none |
| TTP | CD8 | DAP12 | none |
| TTP | CD8 | MyD88 | none |
| TTP | CD8 | CD7 | none |
| TTP | CD4 | CD28 | none |
| TTP | CD4 | CD8 | none |
| TTP | CD4 | CD4 | none |
| TTP | CD4 | b2c | none |
| TTP | CD4 | CD137/41BB | none |
| TTP | CD4 | ICOS | none |
| TTP | CD4 | CD27 | none |
| TTP | CD4 | CD28δ | none |
| TTP | CD4 | CD80 | none |
| TTP | CD4 | CD86 | none |
| TTP | CD4 | OX40 | none |
| TTP | CD4 | DAP10 | none |
| TTP | CD4 | MyD88 | none |
| TTP | CD4 | CD7 | none |
| TTP | CD4 | DAP12 | none |
| TTP | CD4 | MyD88 | none |
| TTP | CD4 | CD7 | none |
| TTP | b2c | CD28 | none |
| TTP | b2c | CD8 | none |
| TTP | b2c | CD4 | none |
| TTP | b2c | b2c | none |
| TTP | b2c | CD137/41BB | none |
| TTP | b2c | ICOS | none |
| TTP | b2c | CD27 | none |
| TTP | b2c | CD28δ | none |
| TTP | b2c | CD80 | none |
| TTP | b2c | CD86 | none |
| TTP | b2c | OX40 | none |
| TTP | b2c | DAP10 | none |
| TTP | b2c | MyD88 | none |
| TTP | b2c | CD7 | none |
| TTP | b2c | DAP12 | none |
| TTP | b2c | MyD88 | none |
| TTP | b2c | CD7 | none |
| TTP | CD137/41BB | CD28 | none |
| TTP | CD137/41BB | CD8 | none |
| TTP | CD137/41BB | CD4 | none |
| TTP | CD137/41BB | b2c | none |
| TTP | CD137/41BB | CD137/41BB | none |
| TTP | CD137/41BB | ICOS | none |
| TTP | CD137/41BB | CD27 | none |
| TTP | CD137/41BB | CD28δ | none |
| TTP | CD137/41BB | CD80 | none |
| TTP | CD137/41BB | CD86 | none |
| TTP | CD137/41BB | OX40 | none |
| TTP | CD137/41BB | DAP10 | none |
| TTP | CD137/41BB | MyD88 | none |
| TTP | CD137/41BB | CD7 | none |
| TTP | CD137/41BB | DAP12 | none |
| TTP | CD137/41BB | MyD88 | none |
| TTP | CD137/41BB | CD7 | none |
| TTP | ICOS | CD28 | none |
| TTP | ICOS | CD8 | none |
| TTP | ICOS | CD4 | none |
| TTP | ICOS | b2c | none |
| TTP | ICOS | CD137/41BB | none |
| TTP | ICOS | ICOS | none |
| TTP | ICOS | CD27 | none |
| TTP | ICOS | CD28δ | none |
| TTP | ICOS | CD80 | none |
| TTP | ICOS | CD86 | none |
| TTP | ICOS | OX40 | none |
| TTP | ICOS | DAP10 | none |
| TTP | ICOS | MyD88 | none |
| TTP | ICOS | CD7 | none |
| TTP | ICOS | DAP12 | none |
| TTP | ICOS | MyD88 | none |
| TTP | ICOS | CD7 | none |
| TTP | ICOS | CD28 | none |
| TTP | ICOS | CD8 | none |
| TTP | ICOS | CD4 | none |
| TTP | ICOS | b2c | none |
| TTP | ICOS | CD137/41BB | none |
| TTP | ICOS | ICOS | none |
| TTP | ICOS | CD27 | none |
| TTP | ICOS | CD28δ | none |
| TTP | ICOS | CD80 | none |
| TTP | ICOS | CD86 | none |
| TTP | ICOS | OX40 | none |
| TTP | ICOS | DAP10 | none |
| TTP | ICOS | MyD88 | none |
| TTP | ICOS | CD7 | none |
| TTP | ICOS | DAP12 | none |
| TTP | ICOS | MyD88 | none |
| TTP | ICOS | CD7 | none |
| TTP | CD27 | CD28 | none |
| TTP | CD27 | CD8 | none |
| TTP | CD27 | CD4 | none |
| TTP | CD27 | b2c | none |
| TTP | CD27 | CD137/41BB | none |
| TTP | CD27 | ICOS | none |
| TTP | CD27 | CD27 | none |
| TTP | CD27 | CD28δ | none |
| TTP | CD27 | CD80 | none |
| TTP | CD27 | CD86 | none |
| TTP | CD27 | OX40 | none |
| TTP | CD27 | DAP10 | none |
| TTP | CD27 | MyD88 | none |
| TTP | CD27 | CD7 | none |
| TTP | CD27 | DAP12 | none |
| TTP | CD27 | MyD88 | none |
| TTP | CD27 | CD7 | none |
| TTP | CD28δ | CD28 | none |
| TTP | CD28δ | CD8 | none |
| TTP | CD28δ | CD4 | none |
| TTP | CD28δ | b2c | none |
| TTP | CD28δ | CD137/41BB | none |
| TTP | CD28δ | ICOS | none |
| TTP | CD28δ | CD27 | none |
| TTP | CD28δ | CD28δ | none |
| TTP | CD28δ | CD80 | none |
| TTP | CD28δ | CD86 | none |
| TTP | CD28δ | OX40 | none |
| TTP | CD28δ | DAP10 | none |
| TTP | CD28δ | MyD88 | none |
| TTP | CD28δ | CD7 | none |
| TTP | CD28δ | DAP12 | none |
| TTP | CD28δ | MyD88 | none |
| TTP | CD28δ | CD7 | none |
| TTP | CD80 | CD28 | none |
| TTP | CD80 | CD8 | none |
| TTP | CD80 | CD4 | none |
| TTP | CD80 | b2c | none |
| TTP | CD80 | CD137/41BB | none |
| TTP | CD80 | ICOS | none |
| TTP | CD80 | CD27 | none |
| TTP | CD80 | CD28δ | none |
| TTP | CD80 | CD80 | none |
| TTP | CD80 | CD86 | none |
| TTP | CD80 | OX40 | none |
| TTP | CD80 | DAP10 | none |
| TTP | CD80 | MyD88 | none |
| TTP | CD80 | CD7 | none |
| TTP | CD80 | DAP12 | none |
| TTP | CD80 | MyD88 | none |
| TTP | CD80 | CD7 | none |
| TTP | CD86 | CD28 | none |
| TTP | CD86 | CD8 | none |
| TTP | CD86 | CD4 | none |
| TTP | CD86 | b2c | none |
| TTP | CD86 | CD137/41BB | none |
| TTP | CD86 | ICOS | none |
| TTP | CD86 | CD27 | none |
| TTP | CD86 | CD28δ | none |
| TTP | CD86 | CD80 | none |
| TTP | CD86 | CD86 | none |
| TTP | CD86 | OX40 | none |
| TTP | CD86 | DAP10 | none |
| TTP | CD86 | MyD88 | none |
| TTP | CD86 | CD7 | none |
| TTP | CD86 | DAP12 | none |
| TTP | CD86 | MyD88 | none |
| TTP | CD86 | CD7 | none |
| TTP | OX40 | CD28 | none |
| TTP | OX40 | CD8 | none |
| TTP | OX40 | CD4 | none |
| TTP | OX40 | b2c | none |
| TTP | OX40 | CD137/41BB | none |
| TTP | OX40 | ICOS | none |
| TTP | OX40 | CD27 | none |
| TTP | OX40 | CD28δ | none |
| TTP | OX40 | CD80 | none |
| TTP | OX40 | CD86 | none |
| TTP | OX40 | OX40 | none |
| TTP | OX40 | DAP10 | none |
| TTP | OX40 | MyD88 | none |
| TTP | OX40 | CD7 | none |
| TTP | OX40 | DAP12 | none |
| TTP | OX40 | MyD88 | none |
| TTP | OX40 | CD7 | none |
| TTP | DAP10 | CD28 | none |
| TTP | DAP10 | CD8 | none |
| TTP | DAP10 | CD4 | none |
| TTP | DAP10 | b2c | none |
| TTP | DAP10 | CD137/41BB | none |
| TTP | DAP10 | ICOS | none |
| TTP | DAP10 | CD27 | none |
| TTP | DAP10 | CD28δ | none |
| TTP | DAP10 | CD80 | none |
| TTP | DAP10 | CD86 | none |
| TTP | DAP10 | OX40 | none |
| TTP | DAP10 | DAP10 | none |
| TTP | DAP10 | MyD88 | none |
| TTP | DAP10 | CD7 | none |
| TTP | DAP10 | DAP12 | none |
| TTP | DAP10 | MyD88 | none |
| TTP | DAP10 | CD7 | none |
| TTP | DAP12 | CD28 | none |
| TTP | DAP12 | CD8 | none |
| TTP | DAP12 | CD4 | none |
| TTP | DAP12 | b2c | none |
| TTP | DAP12 | CD137/41BB | none |
| TTP | DAP12 | ICOS | none |
| TTP | DAP12 | CD27 | none |
| TTP | DAP12 | CD28δ | none |
| TTP | DAP12 | CD80 | none |
| TTP | DAP12 | CD86 | none |
| TTP | DAP12 | OX40 | none |
| TTP | DAP12 | DAP10 | none |
| TTP | DAP12 | MyD88 | none |
| TTP | DAP12 | CD7 | none |
| TTP | DAP12 | DAP12 | none |
| TTP | DAP12 | MyD88 | none |
| TTP | DAP12 | CD7 | none |
| TTP | MyD88 | CD28 | none |
| TTP | MyD88 | CD8 | none |
| TTP | MyD88 | CD4 | none |
| TTP | MyD88 | b2c | none |
| TTP | MyD88 | CD137/41BB | none |
| TTP | MyD88 | ICOS | none |
| TTP | MyD88 | CD27 | none |
| TTP | MyD88 | CD28δ | none |
| TTP | MyD88 | CD80 | none |
| TTP | MyD88 | CD86 | none |
| TTP | MyD88 | OX40 | none |
| TTP | MyD88 | DAP10 | none |
| TTP | MyD88 | MyD88 | none |
| TTP | MyD88 | CD7 | none |
| TTP | MyD88 | DAP12 | none |
| TTP | MyD88 | MyD88 | none |
| TTP | MyD88 | CD7 | none |
| TTP | CD7 | CD28 | none |
| TTP | CD7 | CD8 | none |
| TTP | CD7 | CD4 | none |
| TTP | CD7 | b2c | none |
| TTP | CD7 | CD137/41BB | none |
| TTP | CD7 | ICOS | none |
| TTP | CD7 | CD27 | none |
| TTP | CD7 | CD28δ | none |
| TTP | CD7 | CD80 | none |
| TTP | CD7 | CD86 | none |
| TTP | CD7 | OX40 | none |
| TTP | CD7 | DAP10 | none |
| TTP | CD7 | MyD88 | none |
| TTP | CD7 | CD7 | none |
| TTP | CD7 | DAP12 | none |
| TTP | CD7 | MyD88 | none |
| TTP | CD7 | CD7 | none |
| TTP | BTNL3 | CD28 | none |
| TTP | BTNL3 | CD8 | none |
| TTP | BTNL3 | CD4 | none |
| TTP | BTNL3 | b2c | none |
| TTP | BTNL3 | CD137/41BB | none |
| TTP | BTNL3 | ICOS | none |
| TTP | BTNL3 | CD27 | none |
| TTP | BTNL3 | CD28δ | none |
| TTP | BTNL3 | CD80 | none |
| TTP | BTNL3 | CD86 | none |
| TTP | BTNL3 | OX40 | none |
| TTP | BTNL3 | DAP10 | none |
| TTP | BTNL3 | MyD88 | none |
| TTP | BTNL3 | CD7 | none |
| TTP | BTNL3 | DAP12 | none |
| TTP | BTNL3 | MyD88 | none |
| TTP | BTNL3 | CD7 | none |
| TTP | NKG2D | CD28 | none |
| TTP | NKG2D | CD8 | none |
| TTP | NKG2D | CD4 | none |
| TTP | NKG2D | b2c | none |
| TTP | NKG2D | CD137/41BB | none |
| TTP | NKG2D | ICOS | none |
| TTP | NKG2D | CD27 | none |
| TTP | NKG2D | CD28δ | none |
| TTP | NKG2D | CD80 | none |
| TTP | NKG2D | CD86 | none |
| TTP | NKG2D | OX40 | none |
| TTP | NKG2D | DAP10 | none |
| TTP | NKG2D | MyD88 | none |
| TTP | NKG2D | CD7 | none |
| TTP | NKG2D | DAP12 | none |
| TTP | NKG2D | MyD88 | none |
| TTP | NKG2D | CD7 | none |

Also disclosed are bi-specific CARs. Also disclosed are CARs designed to work only in conjunction with another CAR that binds a different antigen, such as a tumor antigen. For example, in these embodiments, the endodomain of the disclosed CAR can contain only a signaling domain (SD) or a co-stimulatory signaling region (CSR), but not both. The second CAR (or endogenous T-cell) provides the missing signal if it is activated. For example, if the disclosed CAR contains an SD but not a CSR, then the immune effector cell containing this CAR is only activated if another CAR (or T-cell) containing a CSR binds its respective antigen. Likewise, if the disclosed CAR contains a CSR but not a SD, then the immune effector cell containing this CAR is only activated if another CAR (or T-cell) containing an SD binds its respective antigen.

Tumor antigens are proteins that are produced by tumor cells that elicit an immune response, particularly T-cell mediated immune responses. The additional antigen binding domain can be an antibody or a natural ligand of the tumor antigen. The selection of the additional antigen binding domain will depend on the particular type of cancer to be treated. Tumor antigens are well known in the art and include, for example, a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, TIM3, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6, E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, TIM3, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Ra, SSTR2, CD19, TIM3, BCMA, GD2, CLL-1, CA-IX, MUCI, HER2, and any combination thereof.

Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pI85erbB2, pI80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG1 6, TA-90Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

Also disclosed are polynucleotides and polynucleotide vectors encoding the disclosed GSC-specific CARs that allow expression of the GSC-specific CARs in the disclosed immune effector cells.

Nucleic acid sequences encoding the disclosed CARs, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids encoding CARs is typically achieved by operably linking a nucleic acid encoding the CAR polypeptide to a promoter, and incorporating the construct into an expression vector. Typical cloning vectors contain transcription and translation terminators, initiation sequences, and promoters useful for regulation of the expression of the desired nucleic acid sequence.

The disclosed nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodiments, the polynucleotide vectors are lentiviral or retroviral vectors.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

Also disclosed are immune effector cells that are engineered to express the disclosed CARs (also referred to herein as “CAR-T cells.” These cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dentritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4⁺ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (T_{c} cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of α and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can also eradicate MHC-1-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects. Although NK cells have a well-known role as killers of cancer cells, and NK cell impairment has been extensively documented as crucial for progression of MM (Godfrey J, et al. Leuk Lymphoma 2012 53:1666-1676; Fauriat C, et al. Leukemia 2006 20:732-733), the means by which one might enhance NK cell-mediated anti-MM activity has been largely unexplored prior to the disclosed CARs.

Immune effector cells expressing the disclosed CARs can elicit an anti-tumor immune response against GSCs. The anti-tumor immune response elicited by the disclosed CAR-modified immune effector cells may be an active or a passive immune response. In addition, the CAR-mediated immune response may be part of an adoptive immunotherapy approach in which CAR-modified immune effector cells induce an immune response specific to GSCs.

Adoptive transfer of immune effector cells expressing chimeric antigen receptors is a promising anti-cancer therapeutic. Following the collection of a patient's immune effector cells, the cells may be genetically engineered to express the disclosed GSC-specific CARs, then infused back into the patient.

The disclosed CAR-modified immune effector cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat MM. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed CAR-modified immune effector cells are administered to a patient in conjunction with (e.g., before, simultaneously or following) any number of relevant treatment modalities, including but not limited to thalidomide, dexamethasone, bortezomib, and lenalidomide. In further embodiments, the CAR-modified immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an

The disclosed CARs can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed CARs can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MEDI4736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed CARs can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec STI571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with a CARs for treating the disorders as described above may be an anti-cancer cytokine, chemokine, or combination thereof. Examples of suitable cytokines and growth factors include IFNγ, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-Ia from the human CXC and C-C chemokine families. Suitable cytokines include cytokine derivatives, cytokine variants, cytokine fragments, and cytokine fusion proteins.

In some embodiments, a therapeutic agent for use in combination with a CARs for treating the disorders as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent for use in combination with a CARs for treating the disorders as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with a CARs for treating the disorders as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed CARs is administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed CARs is administered in combination with surgery.

CAR-T cells may be designed in several ways that enhance tumor cytotoxicity and specificity, evade tumor immunosuppression, avoid host rejection, and prolong their therapeutic half-life. TRUCK (T-cells Redirected for Universal Cytokine Killing) T cells for example, possess a CAR but are also engineered to release cytokines such as IL-12 that promote tumor killing. Because these cells are designed to release a molecular payload upon activation of the CAR once localized to the tumor environment, these CAR-T cells are sometimes also referred to as ‘armored CARs’. Several cytokines as cancer therapies are being investigated both pre-clinically and clinically, and may also prove useful when similarly incorporated into a TRUCK form of CAR-T therapy. Among these include IL-2, IL-3. IL-4, IL-5, IL-6, IL-7, IL-10, IL-12, IL-13, IL-15, IL-18, M-CSF, GM-CSF, IFN-α, IFN-γ, TNF-α, TRAIL, FLT3 ligand, Lymphotactin, and TGF-β (Dranoff 2004). “Self-driving” or “homing” CAR-T cells are engineered to express a chemokine receptor in addition to their CAR. As certain chemokines can be upregulated in tumors, incorporation of a chemokine receptor aids in tumor trafficking to and infiltration by the adoptive T-cell, thereby enhancing both specificity and functionality of the CAR-T (Moon 2011). Universal CAR-T cells also possess a CAR, but are engineered such that they do not express endogenous TCR (T-cell receptor) or MHC (major histocompatibility complex) proteins. Removal of these two proteins from the signaling repertoire of the adoptive T-cell therapy prevents graft-versus-host-disease and rejection, respectively. Armored CAR-T cells are additionally so named for their ability to evade tumor immunosuppression and tumor-induced CAR-T hypofunction. These particular CAR-Ts possess a CAR, and may be engineered to not express checkpoint inhibitors. Alternatively, these CAR-Ts can be co-administered with a monoclonal antibody (mAb) that blocks checkpoint signaling. Administration of an anti-PDL1 antibody significantly restored the killing ability of CAR TILs (tumor infiltrating lymphocytes). While PD1-PDL1 and CTLA-4-CD80/CD86 signaling pathways have been investigated, it is possible to target other immune checkpoint signaling molecules in the design of an armored CAR-T including LAG-3, Tim-3, IDO-1, 2B4, and KIR. Other intracellular inhibitors of TILs include phosphatases (SHP1), ubiquitin-ligases (i.e., cbl-b), and kinases (i.e., diacylglycerol kinase). Armored CAR-Ts may also be engineered to express proteins or receptors that protect them against or make them resistant to the effects of tumor-secreted cytokines. For example, CTLs (cytotoxic T lymphocytes) transduced with the double negative form of the TGF-β receptor are resistant to the immunosuppression by lymphoma secreted TGF-β. These transduced cells showed notably increased antitumor activity in vivo when compared to their control counterparts.

A tandem CAR contains two sequential antigen binding domains facing the extracellular environment connected to the intracellular costimulatory and stimulatory domains. A dual CAR can be engineered such that one extracellular antigen binding domain is connected to the intracellular costimulatory domain and a second, distinct extracellular antigen binding domain is connected to the intracellular stimulatory domain. Because the stimulatory and costimulatory domains are split between two separate antigen binding domains, dual CARs are also referred to as “split CARs”. In both tandem and dual CAR designs, binding of both antigen binding domains is necessary to allow signaling of the CAR circuit in the T-cell. Because these two CAR designs have binding affinities for different, distinct antigens, they are also referred to as “bi-specific” CARs.

One primary concern with CAR-T cells as a form of “living therapeutic” is their manipulability in vivo and their potential immune-stimulating side effects. To better control CAR-T therapy and prevent against unwanted side effects, a variety of features have been engineered including off-switches, safety mechanisms, and conditional control mechanisms. Both self-destruct and marked/tagged CAR-T cells for example, are engineered to have an “off-switch” that promotes clearance of the CAR-expressing T-cell. A self-destruct CAR-T contains a CAR, but is also engineered to express a pro-apoptotic suicide gene or “elimination gene” inducible upon administration of an exogenous molecule. A variety of suicide genes may be employed for this purpose, including HSV-TK (herpes simplex virus thymidine kinase), Fas, iCasp9 (inducible caspase 9), CD20, MYC TAG, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. A marked/tagged CAR-T cell however, is one that possesses a CAR but also is engineered to express a selection marker. Administration of a mAb against this selection marker will promote clearance of the CAR-T cell. Truncated EGFR is one such targetable antigen by the anti-EGFR mAb, and administration of cetuximab works to promotes elimination of the CAR-T cell. CARs created to have these features are also referred to as sCARs for ‘switchable CARs’, and RCARs for ‘regulatable CARs’. A “safety CAR”, also known as an “inhibitory CAR” (iCAR), is engineered to express two antigen binding domains. One of these extracellular domains is directed against a tumor related antigen and bound to an intracellular costimulatory and stimulatory domain. The second extracellular antigen binding domain however is specific for normal tissue and bound to an intracellular checkpoint domain such as CTLA4, PD1, or CD45. Incorporation of multiple intracellular inhibitory domains to the iCAR is also possible. Some inhibitory molecules that may provide these inhibitory domains include B7-H1, B7-1, CD160, PIH, 2B4, CEACAM (CEACAM-1. CEACAM-3, and/or CEACAM-5), LAG-3, TIGIT, BTLA, LAIR1, and TGFβ-R. In the presence of normal tissue, stimulation of this second antigen binding domain will work to inhibit the CAR. It should be noted that due to this dual antigen specificity, iCARs are also a form of bi-specific CAR-T cells. The safety CAR-T engineering enhances specificity of the CAR-T cell for tumor tissue, and is advantageous in situations where certain normal tissues may express very low levels of a tumor associated antigen that would lead to off target effects with a standard CAR (Morgan 2010). A conditional CAR-T cell expresses an extracellular antigen binding domain connected to an intracellular costimulatory domain and a separate, intracellular costimulator. The costimulatory and stimulatory domain sequences are engineered in such a way that upon administration of an exogenous molecule the resultant proteins will come together intracellularly to complete the CAR circuit. In this way, CAR-T activation can be modulated, and possibly even ‘fine-tuned’ or personalized to a specific patient. Similar to a dual CAR design, the stimulatory and costimulatory domains are physically separated when inactive in the conditional CAR; for this reason these too are also referred to as a “split CAR”.

In some embodiments, two or more of these engineered features may be combined to create an enhanced, multifunctional CAR-T. For example, it is possible to create a CAR-T cell with either dual- or conditional-CAR design that also releases cytokines like a TRUCK. In some embodiments, a dual-conditional CAR-T cell could be made such that it expresses two CARs with two separate antigen binding domains against two distinct cancer antigens, each bound to their respective costimulatory domains. The costimulatory domain would only become functional with the stimulatory domain after the activating molecule is administered. For this CAR-T cell to be effective the cancer must express both cancer antigens and the activating molecule must be administered to the patient; this design thereby incorporating features of both dual and conditional CAR-T cells.

Typically, CAR-T cells are created using α-β T cells, however γ-δ T cells may also be used. In some embodiments, the described CAR constructs, domains, and engineered features used to generate CAR-T cells could similarly be employed in the generation of other types of CAR-expressing immune cells including NK (natural killer) cells, B cells, mast cells, myeloid-derived phagocytes, and NKT cells. Alternatively, a CAR-expressing cell may be created to have properties of both T-cell and NK cells. In an additional embodiment, the transduced with CARs may be autologous or allogeneic.

Several different methods for CAR expression may be used including retroviral transduction (including γ-retroviral), lentiviral transduction, transposon/transposases (Sleeping Beauty and PiggyBac systems), and messenger RNA transfer-mediated gene expression. Gene editing (gene insertion or gene deletion/disruption) has become of increasing importance with respect to the possibility for engineering CAR-T cells as well. CRISPR-Cas9, ZFN (zinc finger nuclease), and TALEN (transcription activator like effector nuclease) systems are three potential methods through which CAR-T cells may be generated.

Also disclosed is a pharmaceutical composition comprising a molecule disclosed herein in a pharmaceutically acceptable carrier. Pharmaceutical carriers are known to those skilled in the art. These most typically would be standard carriers for administration of drugs to humans, including solutions such as sterile water, saline, and buffered solutions at physiological pH. For example, suitable carriers and their formulations are described in Remington: The Science and Practice of Pharmacy (21 ed.) ed. PP. Gerbino, Lippincott Williams & Wilkins, Philadelphia, P A. 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. It will be apparent to those persons skilled in the art that certain carriers may be more preferable depending upon, for instance, the route of administration and concentration of composition being administered.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Also disclosed is a method for treating a lung cancer-derived metastatic brain tumor in a subject by administering to the subject a therapeutically effective amount of the disclosed pharmaceutical composition.

The disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. In some embodiments, the disclosed composition is administered intrathecally.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The compositions disclosed herein may be administered prophylactically to patients or subjects who are at risk for a lung cancer-derived metastatic brain tumor. Thus, the method can further comprise identifying a subject at risk for a lung cancer-derived metastatic brain tumor prior to administration of the herein disclosed compositions.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. A typical daily dosage of the disclosed composition used alone might range from about 1 μg/kg to up to 100 mg/kg of body weight or more per day, depending on the factors mentioned above.

In some embodiments, the molecule is administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of molecule administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Methods

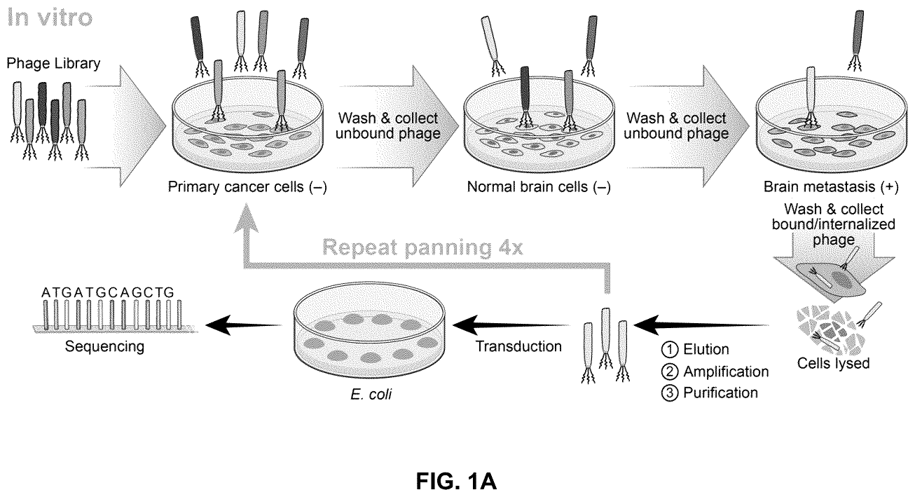

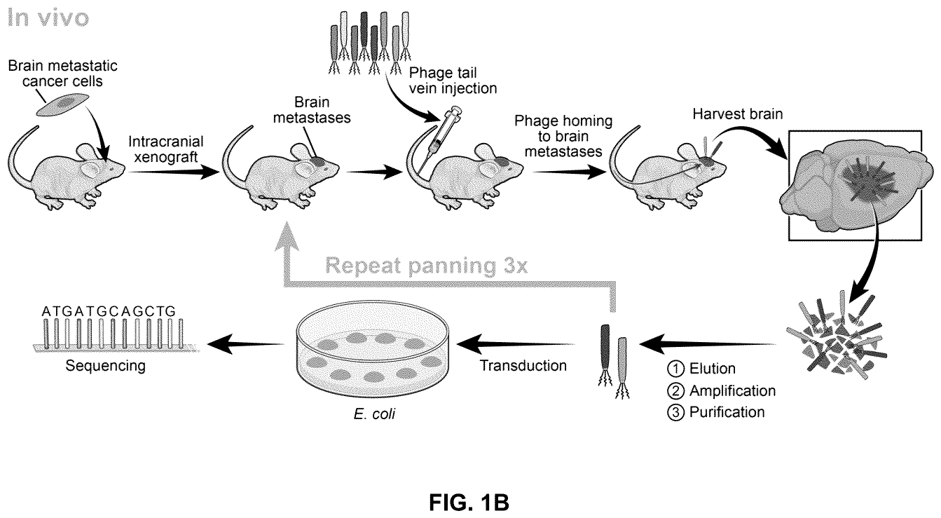

Two strategies were used for phage display screening use to isolate lung cancer derived metastatic brain tumors. FIG. 1A illustrates an in vitro screening method. The phage library (commercially avail through New England Biolabs), was applied to lung cancer cells. Phage peptides that were NOT bound were collected. This is a negative selection process to eliminate peptides that bind lung cancer cells. The collected peptides were applied to normal brain tissue, and again peptides that were NOT bound were collected. The phage library was then applied to lung cancer derived metastatic brain tumor cells. After washing away non-binding peptides, phage peptides were collected and amplified. The amplified peptides were then applied to the primary cancer cells, and this is repeated 4× to enrich for phage peptides that strongly bind lung cancer brain metastases. After 4 rounds, the peptides were collected and sequenced.

In the in vivo strategy (FIG. 1B), NSG mice have lung cancer brain mets injected into the intracranial cavity. After maximal tumor growth, the phage library was injected into the tail vein and after 24 hours, the intracranial tumors were collected and the phage peptides were harvested. This phage peptides were amplified and reapplied to new mice with intracranial tumors for 4 total rounds. After 4 rounds, the peptides were collected and sequenced.

### Results

Table 7 shows all the phage peptides that came up from the in vitro screening process. The right column are all the phage peptides that came up from the in vivo screening process.

TABLE 7

| In vitro Phage Screening | | In vivo Phage Screening | |
|---|---|---|---|
| D04 | ADSNHAYERDSVGGG (SEQ ID NO: 85) | B06 | AHPHSDKLVPPRGGGS (SEQ ID NO: 122) |
| D01 | AESPLTNRGWNPGGG (SEQ ID NO: 86) | B07 | AHSSFGFSHLAVGGGS (SEQ ID NO: 123) |
| F03 | AHVNSSERIHPYGGG (SEQ ID NO: 87) | F03 | AMNMRSLTYSDLGGGS (SEQ ID NO: 124) |
| F02 | ANTELALANRKHGGG (SEQ ID NO: 88) | D05 | APQTSDSTLTVMGGGS (SEQ ID NO: 125) |
| D06 | APNVGDLTSLLGGGG (SEQ ID NO: 89) | A07 | EALLKPYSFAYPGGGS (SEQ ID NO: 126) |
| A03 | AQSTTMSTSQRNGGG (SEQ ID NO: 90) | A05 | EHVDMWNLVSASGGGS (SEQ ID NO: 127) |
| B02 | ARSLEPAPSRHSGGG (SEQ ID NO: 91) | G01 | EKYPPSSMDQRSGGGS (SEQ ID NO: 128) |
| B03 | ATPGSFQTIRANGGG (SEQ ID NO: 92) | D01 | FPKSVYPAMPRPGGGS (SEQ ID NO: 129) |
| C03 | | G02 | FSPTQANTIHRWGGGS (SEQ ID NO: 130) |
| A04 | CVTPSNRDCSHSGGG (SEQ ID NO: 93) | G04 | |
| H04 | ELVSSSDQRNKNGGG (SEQ ID NO: 94) | H02 | |
| G03 | GPWLGSNMRGASGGG (SEQ ID NO: 95) | H05 | FTNPFGPYPTGRGGGS (SEQ ID NO: 131) |
| E01 | GSAARTISPSLLGGG (SEQ ID NO: 96) | A04 | GKQTLHSFATHWGGGS (SEQ ID NO: 132) |
| F04 | GSMFYLPMPERGGGG (SEQ ID NO: 97) | F04 | GRDMPMSALMRHGGGS (SEQ ID NO: 133) |
| A01 | GTASRTHSYYSLGGG (SEQ ID NO: 98) | A06 | GTDIIHPRVIFNGGGS (SEQ ID NO: 134) |
| A06 | | E06 | |
| C05 | HPALSQHLGPVAGGG (SEQ ID NO: 99) | F02 | HNTPMLDSRGNNGGGS (SEQ ID NO: 135) |
| A02 | HTPHPGGRSVPPGGG (SEQ ID NO: 100) | E02 | HSPTTSLTLSYKGGGS (LBM4, SEQ ID NO: 136) |
| E05 | IGSKSPLRLTMDGGG (SEQ ID NO: 101) | G03 | |
| D05 | KLTTDKVRTITLGGG (SEQ ID NO: 102) | B02 | HYKPHVSSLPLAGGGS (SEQ ID NO: 137) |
| G05 | LIAGWDISSKRGGG (SEQ ID NO: 103) | B01 | LTPHLGTHKSTTGGGS (SEQ ID NO: 138) |
| C04 | LPKVSVPRHPSVGGG (SEQ ID NO: 104) | F05 | MNIAELRNSDLNGGGS (SEQ ID NO: 139) |
| B01 | MKAHHSQLYPRHGGG (SEQ ID NO: 105) | D06 | N/A |
| C01 | | E04 | N/A |
| G04 | | G06 | N/A |
| C02 | N/A | G05 | NANHNPLNLKSAGGGS (SEQ ID NO: 140) |
| B04 | N/A | A03 | QFAKTSDPGSLTGGGS (SEQ ID NO: 141) |
| B06 | N/A | F06 | QIFNHSPNDPKKGGGS (SEQ ID NO: 142) |
| C06 | N/A | A02 | RIPMPSYMNHMGGGS (SEQ ID NO: 143) |
| H06 | N/A | H01 | SDASGIPKRLAFPGGS (SEQ ID NO: 144) |
| H02 | QYVPYLPPAILGGG (SEQ ID NO: 106) | A01 | SDTTSKHLYTRLGGGS (SEQ ID NO: 145) |
| F01 | NNTDHRQLTSTTGGG (SEQ ID NO: 107) | C07 | SGYSAEGGKPVLGGGS (SEQ ID NO: 146) |
| D02 | NYLPHQSSSPSRGGG (SEQ ID NO: 108) | H03 | |
| H02 | QYVPYLPPAILGGG (SEQ ID NO: 109) | B05 | SGYSQGGKPVLGGGS (SEQ ID NO: 147) |
| F05 | SNPSAHKDDSKRGGG (SEQ ID NO: 110) | E01 | SHGISSTPPGQAGGGS (SEQ ID NO: 148) |
| H01 | SRYMGPLDLLAPGGG (SEQ ID NO: 111) | B04 | SLPLAIHNSRPNGGGS (SEQ ID NO: 149) |
| F06 | SRYMGPLDLLAPGGG (SEQ ID NO: 111) | C01 | SLTDYVRKGPRIGGGS (SEQ ID NO: 150) |
| G06 | SVDYSFSNRTDRGGG (SEQ ID NO: 112) | E03 | SSMPINSPATRQGGGS (SEQ ID NO: 151) |
| G01 | SYHLSNTFRVQAGGG (SEQ ID NO: 113) | F01 | SYPSNALSLHKYGGGS (LBM1, SEQ ID NO: 152) |
| D03 | SYPSNALSLHKYGGG (SEQ ID NO: 114) | C03 | TAVLAPQPWLNLGGGS (SEQ ID NO: 153) |
| A05 | TLGLRPVPVATTGGG (SEQ ID NO: 115) | D04 | TGSAKFLQRDTHGGGS (SEQ ID NO: 154) |
| H05 | TTDFFYKRTFFTGGG (SEQ ID NO: 116) | H06 | TKPSVVHWPMIRGGGS (SEQ ID NO: 155) |
| E03 | TTRPNHVHLAKIGGG (SEQ ID NO: 117) | H04 | TLGLRPVPVATTGGGS (LBM2, SEQ ID NO: 156) |
| E04 | VAQRHTLTSITVGGG (SEQ ID NO: 118) | C06 | TPHGYQPMQGKTGGGS (SEQ ID NO: 157) |
| H03 | VPTQWTHRESHAGGG (SEQ ID NO: 119) | C05 | TQGSGFSSPILPGGGS (SEQ ID NO: 158) |
| B05 | WGVTKPIRTSTLGGG (SEQ ID NO: 120) | D02 | VDAKYGRHIPPVGGGS (SEQ ID NO: 159) |
| E06 | WGVTKPIRTSTLGGG (SEQ ID NO: 120) | C02 | WYPSNALSLHKGGGS (SEQ ID NO: 160) |
| E02 | YDAIQRPTGQLSGGG (SEQ ID NO: 121) | B03 | YASQSALSHSARGGGS (SEQ ID NO: 161) |
| | | C04 | YDAIQRPTGQLSGGGS (LBM3, SEQ ID NO: 162) |
| | | D03 | |
| | | E05 | YVKSHTTTAVRQGGGS (SEQ ID NO: 163) |

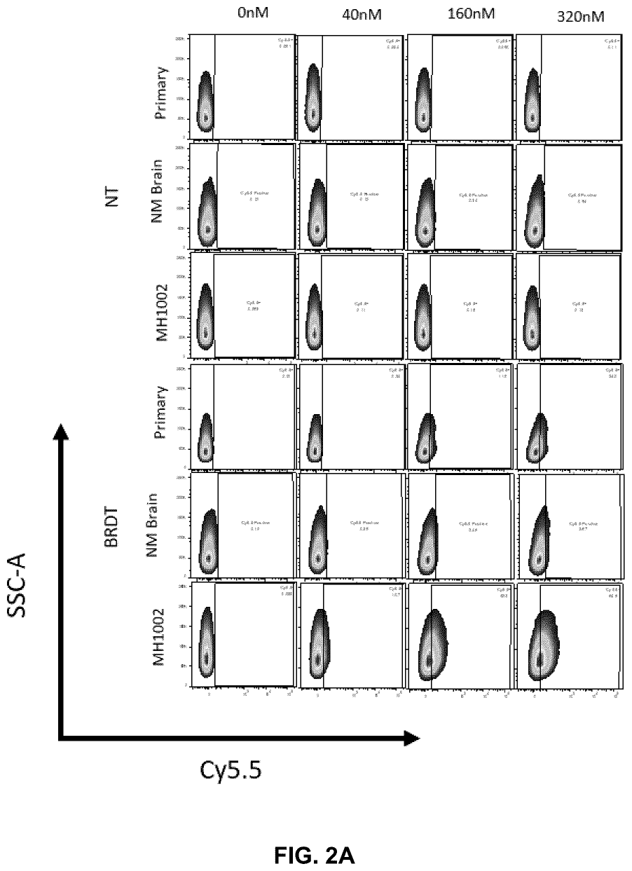

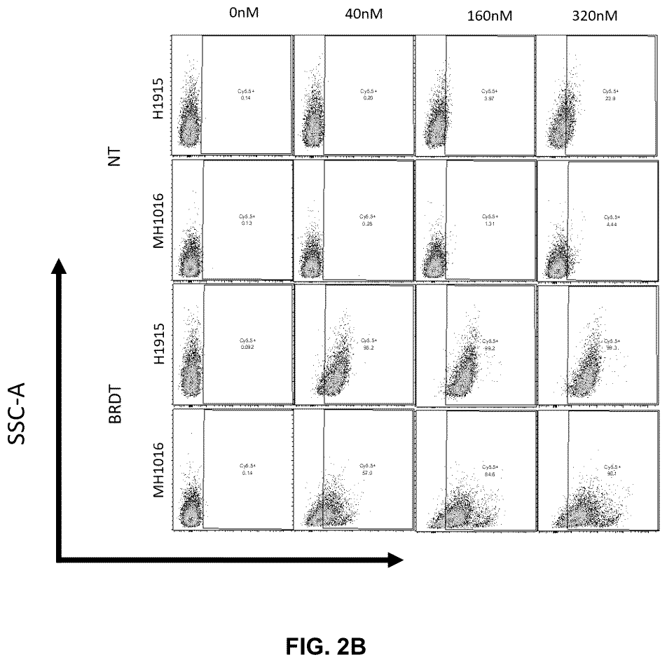

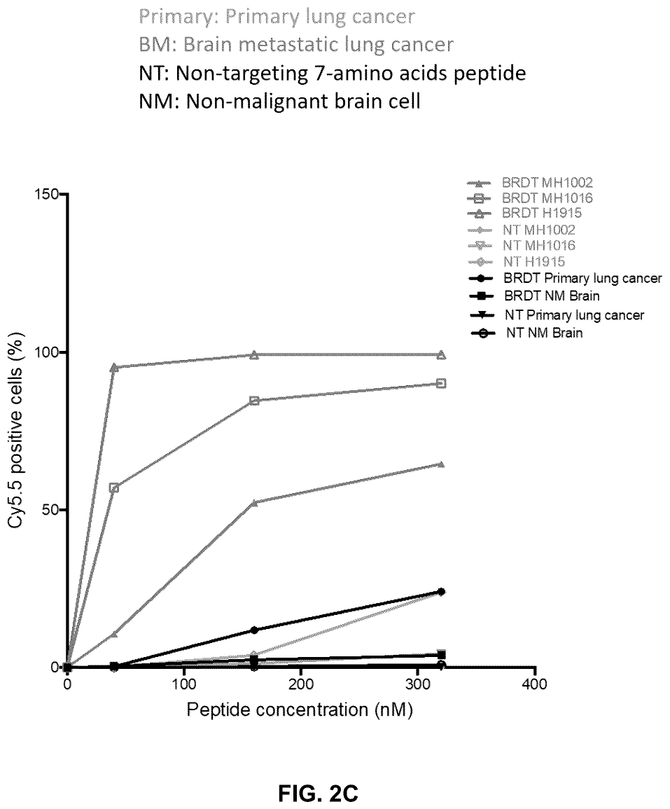

FIGS. 2A to 2C show binding of BRDT (LBM1) peptide applied to Primary Lung Cancer Cells, Non Malignant Brain Cells, Lung Cancer Brain Metastases Cells (MH1002, MH1016, H1915) analyzed through flow cytometry. Flow cytometry demonstrated right shift with BRDT peptide applied to lung cancer BM cells when compared to primary lung cancer cells and non-targeting peptides.

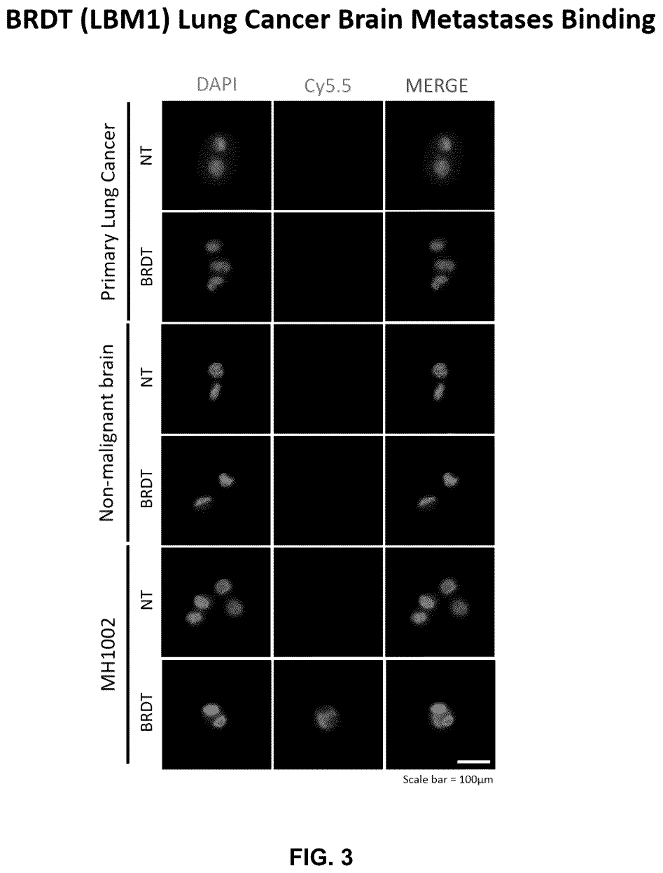

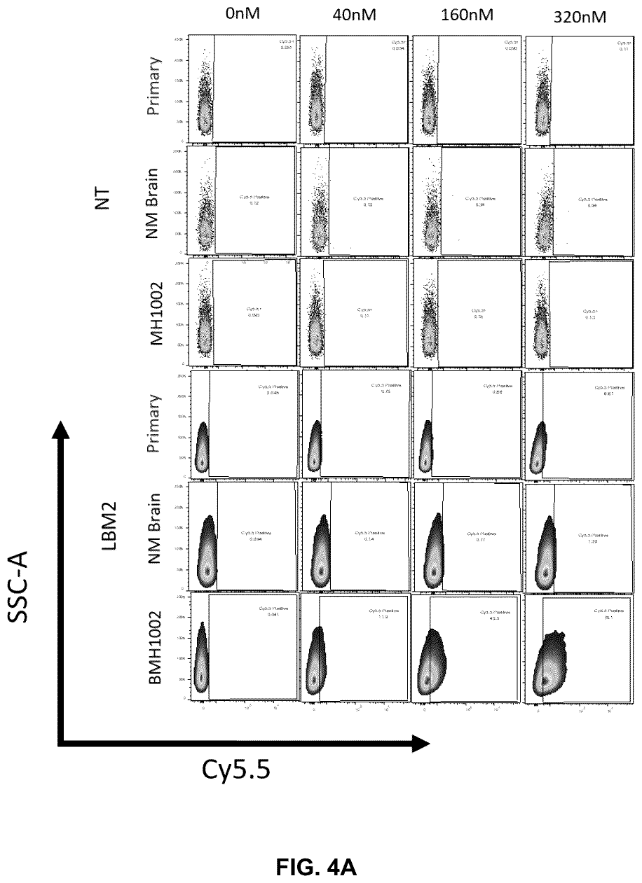

FIG. 3 shows positive binding of BRDT (LBM1) peptide applied to Lung Cancer Brain Metastases cells (MH1002). LBM1 peptide applied to primary lung cancer cells (Primary) and non-malignant brain cells shows no binding.

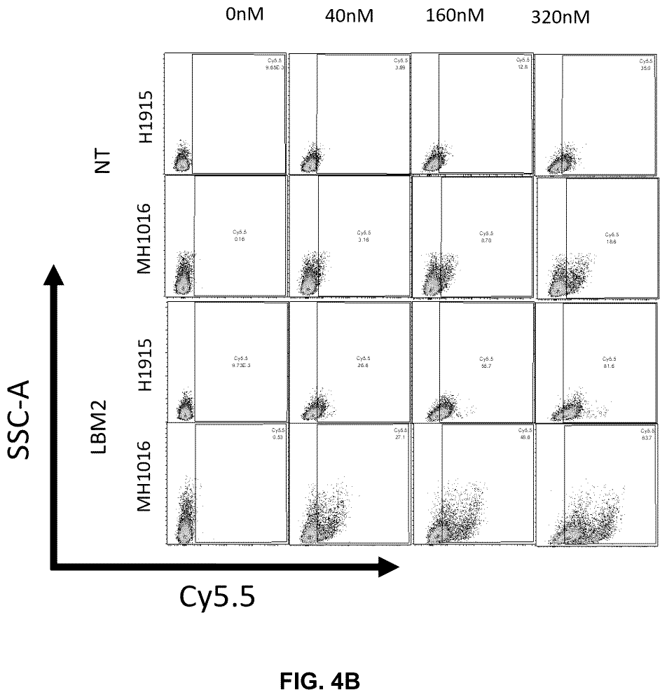

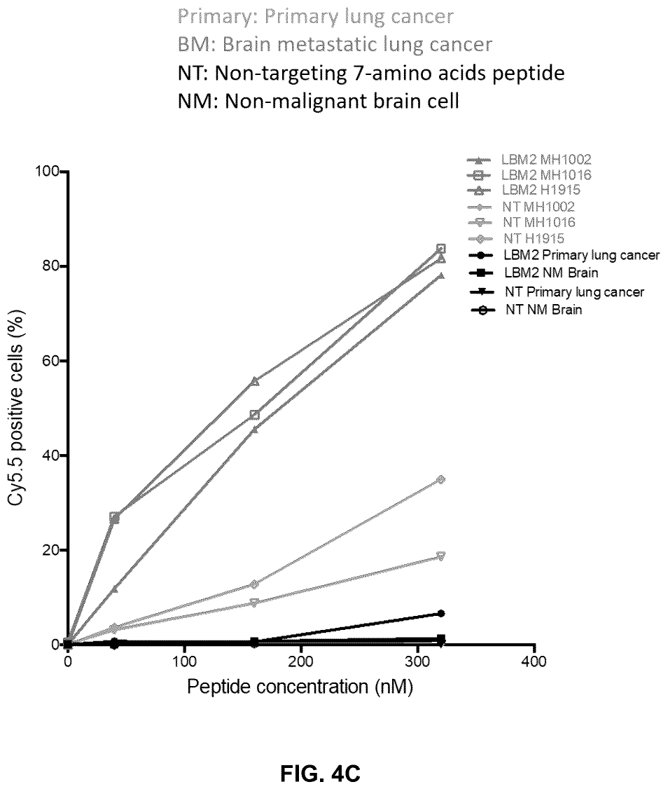

FIGS. 4A to 4C show binding of LBM2 peptide applied to Primary Lung Cancer Cells, Non Malignant Brain Cells, Lung Cancer Brain Metastases Cells (MH1002, MH1016, H1915) analyzed through flow cytometry. Flow cytometry demonstrated right shift with LBM2 peptide applied to lung cancer BM cells when compared to primary lung cancer cells and non-targeting peptides.

FIG. 4 shows positive binding of LBM2 peptide applied to Lung Cancer Brain Metastases cells (MH1002). LBM2 peptide applied to primary lung cancer cells (Primary) and non-malignant brain cells shows no binding.

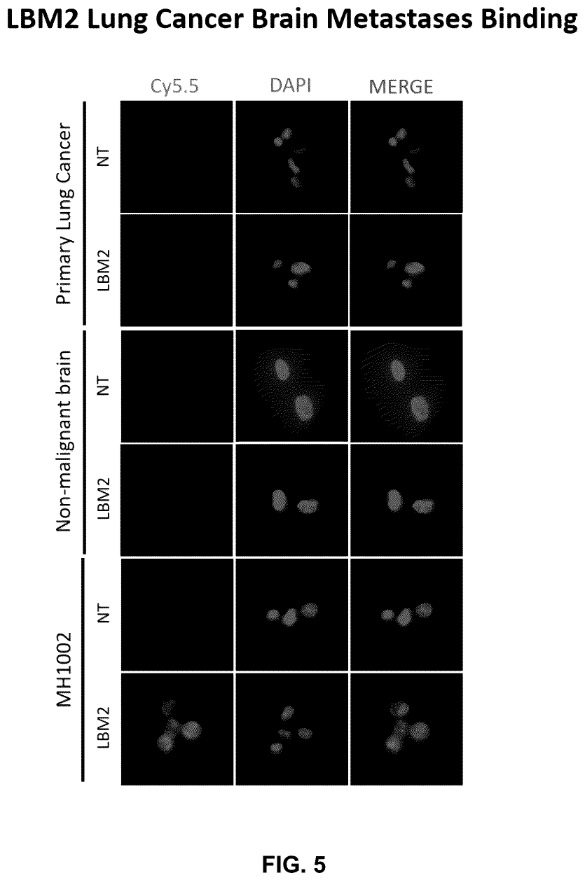

FIGS. 5A to 5C show binding AMOT (LBM4) peptide applied to Primary Lung Cancer Cells, Non Malignant Brain Cells, Lung Cancer Brain Metastases Cells (MH1002, MH1016, H1915) analyzed through flow cytometry. Flow cytometry demonstrated right shift with AMOT peptide applied to lung cancer BM cells when compared to primary lung cancer cells and non-targeting peptides.

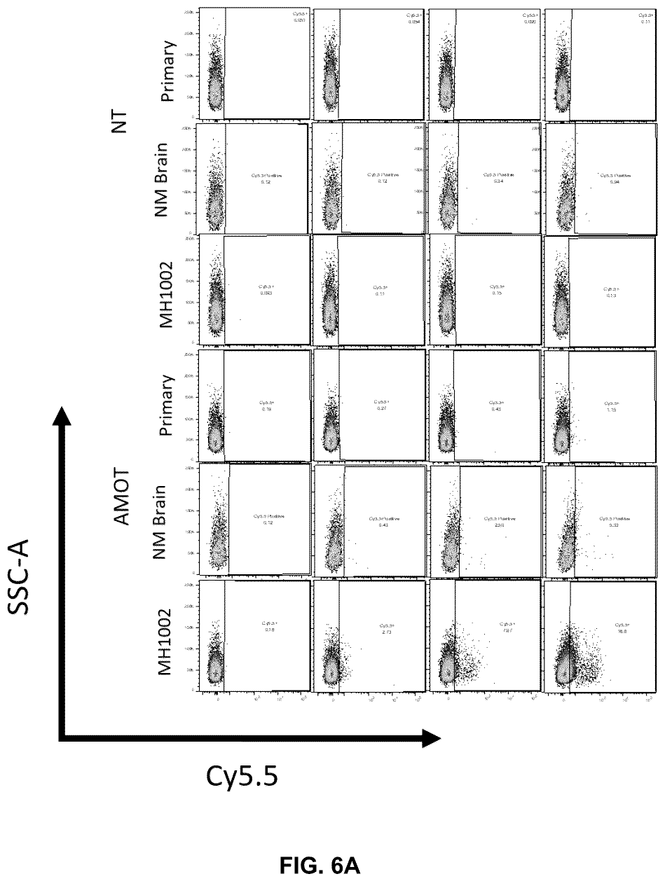

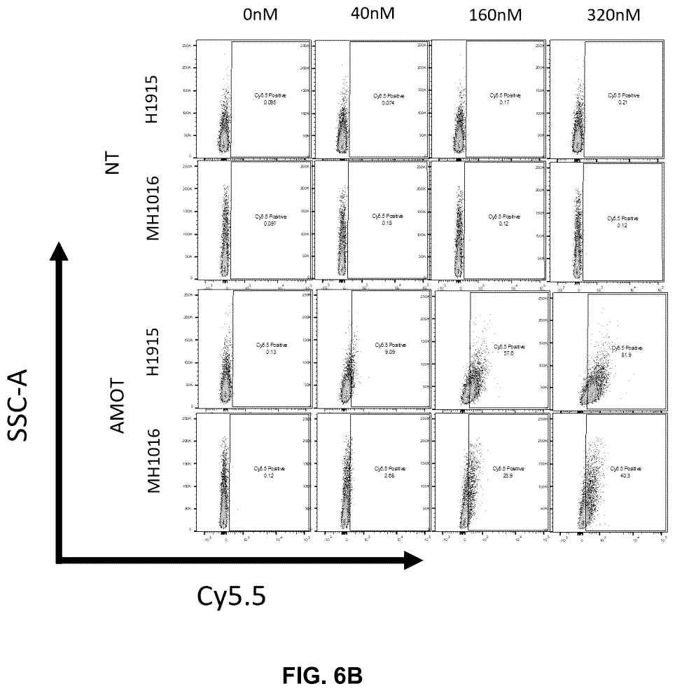

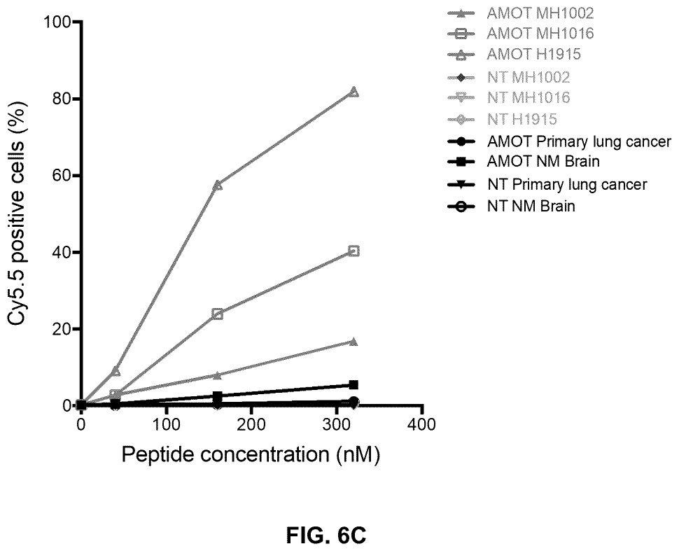

FIG. 6A shows no binding of AMOT (LBM4) peptide applied to primary lung cancer cells and non-malignant brain cells. FIG. 6B shows positive binding of AMOT peptide applied to Lung Cancer Brain Metastases cells (M1002, M1016, H1915).

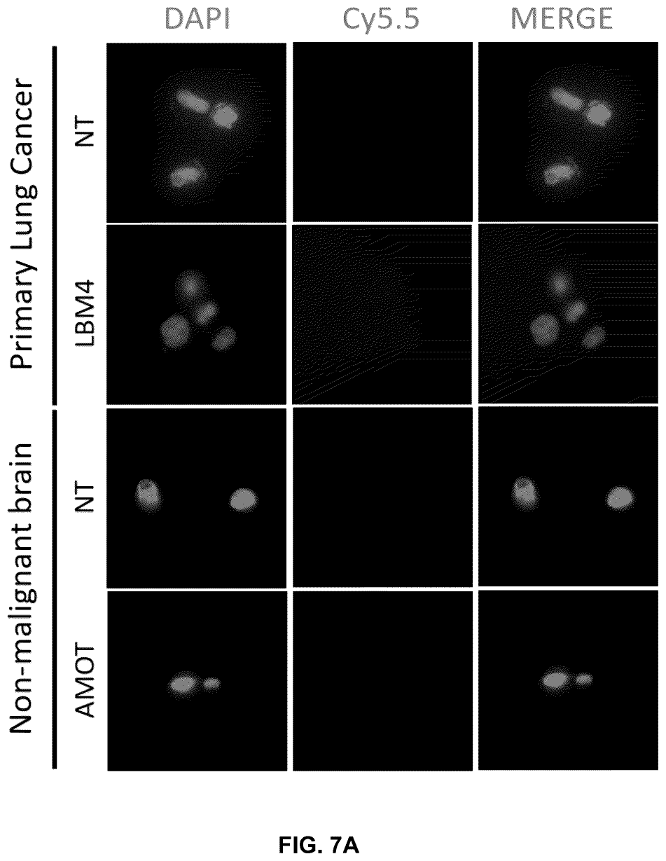

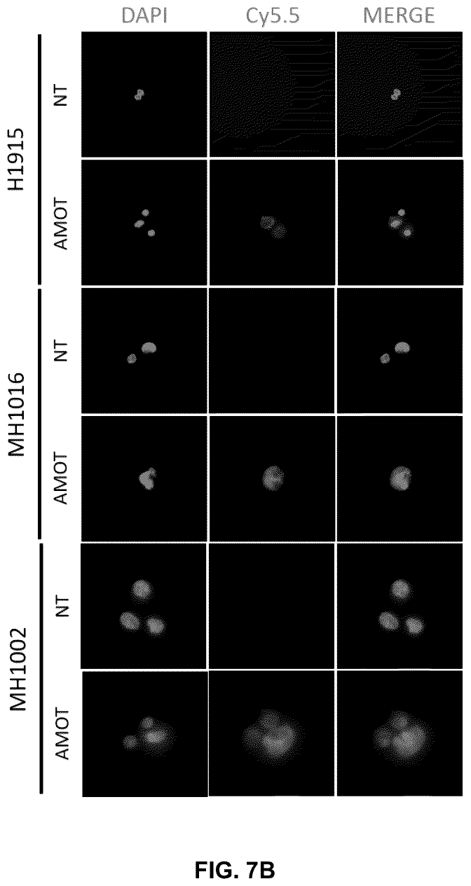

FIGS. 7A and 7B show peptide signal time course. Mice were 29 days post implantation of intracranial lung cancer brain metastatic tumor. 400 μM of LBM4 peptide was injected via tail vein. Mice were imaged at 0, 3, 6, 12, 24, and 48 hours after initial injection. Graph demonstrate signal height at 3 hours with gradual decrease. NT=non-targeting peptide (non-specific peptide).

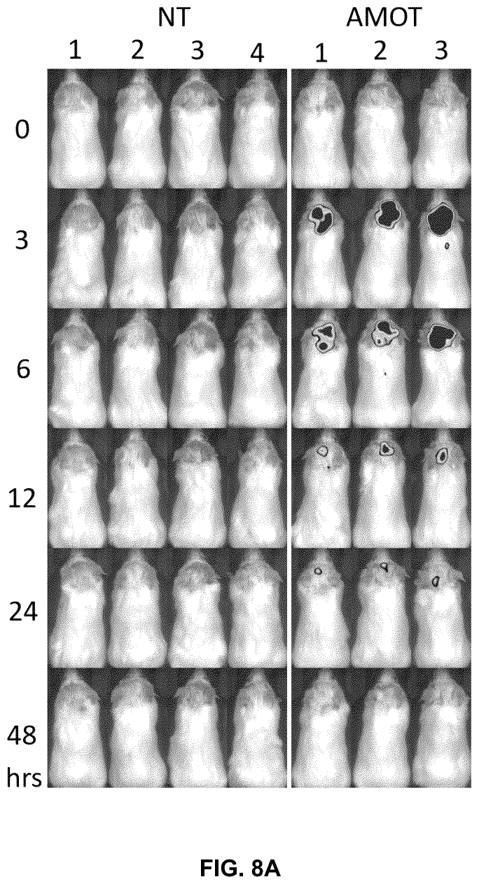

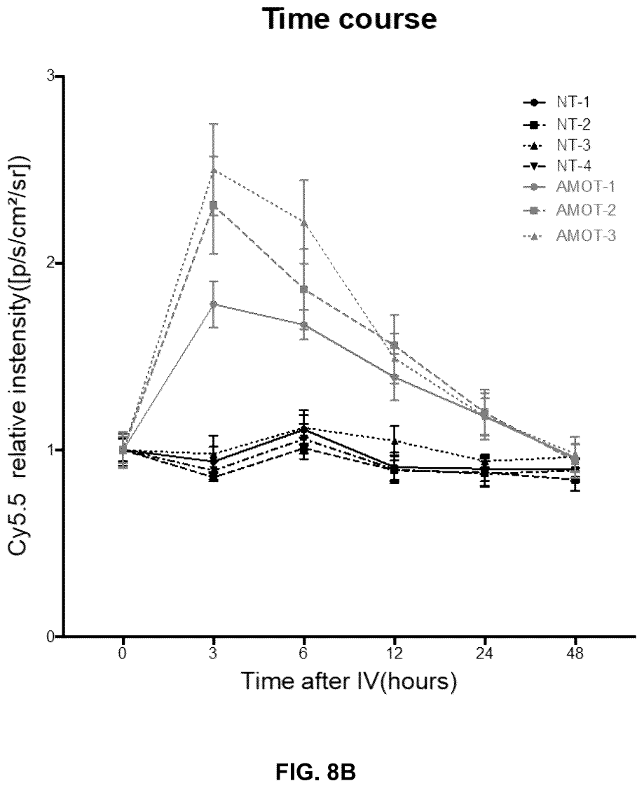

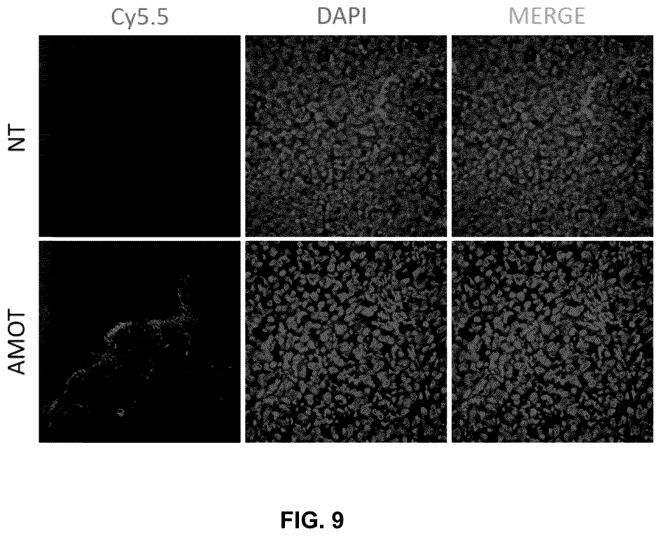

FIG. 8 shows binding of 400 uM peptide injected into the tail vein of mice with intracranial implanted lung cancer brain metastasis. Cy5.5 visualizes peptide binding.

For each peptide, as the concentration of peptide goes up, there was a right shift indicating binding to the brain met cells, with minimal binding to the lung cancer primary cells, showing specificity of binding to the brain mets over the lung cancer.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A chimeric antigen receptor (CAR) polypeptide, comprising a tumor targeting peptide, a transmembrane domain, an intracellular signaling domain, and a co-stimulatory signaling region, wherein the tumor targeting peptide comprises the amino acid sequence SEQ ID NO:1, SEQ ID NO:2, or SEQ ID NO:4, or a variant having at least 10, 11, 12, 13, 14, or 15 contiguous amino acids and at least 65%, 70%, 75, 80%, 85%, 90%, 95%, or 100% sequence identity to SEQ ID NO:1, SEQ ID NO:2, or SEQ ID NO:4.

2. The polypeptide of claim 1, wherein the costimulatory signaling region comprises the cytoplasmic domain of a costimulatory molecule selected from the group consisting of CD27, CD28, 4-11BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

3. The polypeptide of claim 1, wherein the CAR polypeptide is defined by the formula:
SP-TTP-HG-TM-CSR-ISD; or
SP-TTP-HG-TM-ISD-CSR
wherein “SP” represents a signal peptide,
wherein “TTP” represents the tumor targeting peptide,
wherein “HG” represents and optional hinge domain,
wherein “TM” represents a transmembrane domain,
wherein “CSR” represents a co-stimulatory signaling region,
wherein “ISD” represents an intracellular signaling domain, and
wherein “-” represents a bivalent linker.

4. The polypeptide of claim 1, wherein the intracellular signaling domain comprises a CD3 zeta (CD3ζ) signaling domain.

5. An isolated nucleic acid sequence encoding the recombinant polypeptide of claim 1.

6. A vector comprising the isolated nucleic acid sequence of claim 5.

7. A cell comprising the vector of claim 6.

8. The cell of claim 7, wherein the cell is selected from the group consisting of an αβT cell, γδT cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

9. The cell of claim 8, wherein the cell exhibits an anti-tumor immunity when the antigen binding domain of the CAR binds to a lung cancer-derived metastatic brain tumor.

10. A method of providing an anti-cancer immunity in a subject with lung cancer-derived metastatic brain tumor, the method comprising administering to the subject an effective amount of an immune effector cell genetically modified to express the CAR polypeptide of claim 1, thereby providing an anti-tumor immunity in the subject.

11. The method of claim 10, wherein the immune effector cell is selected from the group consisting of a T cell, a Natural Killer (NK) cell, a cytotoxic T lymphocyte (CTL), and a regulatory T cell.

12. The method of claim 10, further comprising administering to the subject a checkpoint inhibitor.

13. The method of claim 12, wherein the checkpoint inhibitor comprises an anti-PD-1 antibody, anti-PD-L1 antibody, anti-CTLA-4 antibody, or a combination thereof.
